(19) United States
(12) Patent Application Publication
(10) Pub. No.: US 2024/0200095 A1
(43) Pub. Date: Jun. 20, 2024

(54) PROMOTERS

(71) Applicant: FREELINE THERAPEUTICS LIMITED, Hertfordshire (GB)

(72) Inventors: Azadeh KIA, Hertfordshire (GB); Samantha CORREIA, Hertfordshire (GB)

(21) Appl. No.: 18/394,036

(22) Filed: Dec. 22, 2023

Related U.S. Application Data
(63) Continuation of application No. PCT/GB2022/051622, filed on Jun. 24, 2022.

(30) Foreign Application Priority Data
Jun. 25, 2021 (GB) 2109231.7

(51) Int. Cl. C12N 15/86; C07K 14/47; C07K 14/81; C12N 9/06

(52) U.S. Cl. CPC C12N 15/86; C07K 14/4717; C07K 14/8125; C12N 9/002; C12N 2750/14143; C12Y 401/02013

(57) ABSTRACT

The present invention relates to transcription regulatory elements (TREs) based on the combination of a hAAT sequence element and an AMBP sequence element and which promote greater expression than HCR-hAAT. The invention further relates to compositions comprising the AAV vectors, as well as methods of gene therapy based on the use of such vectors and compositions.

### CROSS REFERENCE

This application is a continuation of International Application No. PCT/GB2022/051622, filed Jun. 24, 2022, which claims the benefit of United Kingdom Application No. GB2109231.7, filed Jun. 25, 2021, all of which are herein incorporated by reference in their entirety.

### SEQUENCE LISTING

The instant application contains a Sequence Listing which has been submitted electronically in XML format and is hereby incorporated by reference in its entirety. Said XML copy, created on Dec. 22, 2023, is named 52186-737-301_SL.xml and is 148,700 bytes in size.

### FIELD OF THE INVENTION

The present invention relates to a transcription regulatory element (TRE) that promotes greater expression than HCR-hAAT, promotes greater expression than FRE72, comprises a hAAT sequence element and/or comprises an AMBP sequence element.

### BACKGROUND TO THE INVENTION

Gene therapy represents a promising new field in the treatment of genetic disorders, such as Haemophilia B. Gene therapy utilises viral vectors, such as adeno-associated virus (AAV), to deliver a transgene to a patient suffering from a genetic disorder. This transgene may form part of an expression cassette, in which it is operably linked to a transcription regulatory element (TRE) which promotes endogenous expression of said transgene in a patient. When using a viral vector such as AAV as a gene therapy vector, the construct used may be limited by the size of the native viral genome. For example, AAV viral vectors considerably smaller or larger than the native AAV genome size of 4.7 kbp may be more difficult to produce, less stable and not be capable of as high functional transduction rates compared to AAV viral vectors closer in size to 4.7 kbp. Expression cassettes comprising a TRE operably linked to a transgene for use in different types of viral vectors may therefore benefit from being of a size which is known to be optimal in terms of manufacturability and potency for the respective vector types.

Some transgenes (such as b-domain-deleted FVIII) are close in size to the 4.73 kbp genome of AAV, and so if AAV is to be used to deliver these transgenes, it may be desirable to use very small promoters in the viral vectors. WO2021/084277 discloses a group of excellent small promoters including FRE72. However, some transgenes (like the CFI transgene of SEQ ID NO: 41) are quite small. In these cases, it is often considered desirable to add a “stuffer” sequence, i.e. a stretch of “inert” non-coding nucleotides, to increase the size of the expression cassette. However, stuffer sequences may have unpredictable effects which may lead to a reduction in the efficiency of production, stability and transduction rates of the viral vector.

The present application provides a panel of TREs of different sizes which may promote greater expression than HCR-hAAT, promote greater expression than FRE72, comprise a hAAT sequence element and/or comprise an AMBP sequence element. The present inventors surprisingly found that the addition of various promoter and/or enhancer sequence elements to a hAAT-based promoter or an AMBP-based promoter can provide TREs of various sizes which promote greater expression than HCR-hAAT or FRE72. The differently-sized TREs of the invention can be paired with transgenes of different sizes. Such TREs can therefore be useful in a variety of applications. For example, vectors comprising such TREs operably linked to a transgene may be particularly useful when administered as part of a gene therapy.

### SUMMARY OF THE INVENTION

The present application demonstrates that a panel of TREs of different lengths comprising a hAAT sequence element and/or an AMBP sequence element promote greater expression than HCR-hAAT. The present application also demonstrates that TREs comprising additional promoter or enhancer sequence elements, such as ApoE-HCR1 sequence elements, ALDOB sequence elements, CRM6 sequence elements and/or F2 sequence elements, can be added to TREs comprising AMBP and/or hAAT sequence elements to increase the length of the TREs and/or contribute to promotion of greater expression than HCR-hAAT and FRE72. The present application therefore provides a panel of TREs of different lengths which are capable of promoting greater expression than HCR-hAAT and FRE72.

Accordingly, in the first aspect of the invention, there is provided a transcription regulatory element (TRE) that:
(a) promotes greater expression than HCR-hAAT:
(b) promotes greater expression than FRE72;
(c) comprises a hAAT sequence element; and/or
(d) comprises an AMBP sequence element.

In a second aspect of the invention, there is provided an expression cassette comprising a TRE of the invention operably linked to a transgene.

In a third aspect of the invention, there is provided a viral particle comprising a TRE of the invention or an expression cassette of the invention.

In a fourth aspect of the invention, there is provided an AAV vector comprising a TRE of the invention or an expression cassette of the invention.

In a fifth aspect of the invention, there is provided a pharmaceutical composition comprising a viral particle of the invention or an AAV vector of the invention and a pharmaceutically acceptable excipient.

In a sixth aspect of the invention, there is provided a method of treatment comprising administering an effective amount of the viral particle of the invention or an AAV vector of the invention to a patient.

In a seventh aspect of the invention, there is provided a use of the viral particle, AAV vector or composition of the invention in the manufacture of a medicament for treating a disease.

In an eighth aspect of the invention there is provided the viral particle, AAV vector or composition of the invention for use in a method of treating a disease.

### DESCRIPTION OF THE FIGURES

FIG. 1—Schematic showing the different sequence elements present in FRE72 (SEQ ID NO: 1), HCR-hAAT (SEQ ID NO: 2), NP1 (SEQ ID NO: 3), NP2 (SEQ ID NO: 4), NP3 (SEQ ID NO: 5), NP4 (SEQ ID NO: 6), NP5 (SEQ ID NO: 7), NP6 (SEQ ID NO: 8), and NP7 (SEQ ID NO: 9). The labels in each section of the schematics show the names of the various sequence elements. The sequence element referred to as ApoE-HCR1 is a sequence element of SEQ ID NO: 27. The sequence element referred to as hAAT is a sequence element of SEQ ID NO: 29. The sequence element referred to as AMBP is a sequence element of SEQ ID NO: 32. The sequence element referred to as CRM6 is a sequence element of SEQ ID NO: 34. The sequence element referred to as ALDOB is a sequence element of SEQ ID NO: 37. The sequence element referred to as F2 is a sequence element of SEQ ID NO: 39. The labels to the left of the schematics show the name of the TREs. The numbers to the right of the schematic show the length of the corresponding TREs.

FIG. 2—(a) Schematic showing the sequence elements present in NP3 (SEQ ID NO: 5), including the location of the ApoE enhancer within an ApoE HCR-1 sequence element and the High Homology region within a hAAT sequence element. The scale above shows the nucleotide position of each of the different elements. (b) Table summarising the features of a group of TREs based on NP3 (NP3-derived TREs). The table indicates the differences between various NP3-derived TREs and NP3.

FIG. 3—Graph showing the fold change in expression levels of CFI promoted by NP1, NP2, NP3, NP4, NP5, NP6, and NP7 in Huh7 cells compared to HCR-hAAT. HLP2 and FRE72 are included as controls. Huh7 human hepatocytes were transfected with plasmid DNA comprising TREs operably linked to CFI alongside a control plasmid comprising a luciferase encoding nucleotide sequence operably linked to a CMV promoter. Cells were then grown, harvested and the expression levels of CFI measured by ELISA. In parallel to the ELISA, a luciferase assay was performed to determine luciferase expression levels. CFI expression levels for each sample were normalised against the corresponding luciferase expression levels. Error bars show the mean+1 SD.

FIG. 4—Graph showing the fold change in expression levels of CFI promoted by NP12-NP24 in Huh7 cells compared to HCR-hAAT. HLP2, FRE72, and NP3 are included as controls. Huh7 human hepatocytes were transfected with plasmid DNA comprising TREs operably linked to CFI alongside a control plasmid comprising a luciferase encoding nucleotide sequence operably linked to a CMV promoter. Cells were then grown, harvested and the expression levels of CFI measured by ELISA. In parallel to the ELISA, a luciferase assay was performed to determine luciferase expression levels. CFI expression levels for each sample were normalised against the corresponding luciferase expression levels. Error bars show the mean+/−1 SD.

FIG. 5—Graph showing the fold change in expression levels of CFI promoted by NP8-NP11 in Huh7 cells compared to HCR-hAAT. HLP2, FRE72, and NP3 are included as controls. Huh7 human hepatocytes were transfected with plasmid DNA comprising TREs operably linked to CFI alongside a control plasmid comprising a luciferase encoding nucleotide sequence operably linked to a CMV promoter. Cells were then grown, harvested and the expression levels of CFI measured by ELISA. In parallel to the ELISA, a luciferase assay was performed to determine luciferase expression levels. CFI expression levels for each sample were normalised against the corresponding luciferase expression levels. Error bars show the mean+/−1 SD.

FIG. 6—Sequence Listing.

FIG. 7—Graph showing the percentage of GFP positive cells from Huh7, HEK293T, MCF-7, BxPC3, 1643, MRC-9, 697, and AC-16 cell lines each transduced with vectors (MOI 1×105) comprising GFP operably linked to one of the NP1, NP2, NP6, NP7 and CAG promoters. Cells were transduced with the vectors, grown, and GFP percentage positivity measured by FACS. Error bars show the mean+/−1 SD. In each dataset, e.g. the NP1 dataset on the far left of the graph, from left to right the columns show expression levels in Huh7 cells, HEK293T cells, MCF-7 cells, BxPC3 cells, 1643 cells, MRC-9 cells, 697 cells, and AC-16 cells.

FIG. 8—Graph showing the MFI of cells from Huh7, HEK293T, MCF-7, BxPC3, 1643, MRC-9, 697, and AC-16 cell lines each transduced with AAV vectors comprising GFP operably linked to one of the NP1, NP2, NP6, NP7 and CAG promoters. Cells were transduced with the vectors, grown, and MFI at 488 nm measured by FACS. The MFI for each of the duplicates is shown along with the median. In each dataset, e.g. the Huh7 dataset on the far left of the graph, from left to right the data points show expression levels promoted by the CAG promoter, NP7 promoter, NP6 promoter, NP2 promoter, NP1 promoter, and the untransduced control cells.

FIG. 9—Graph showing the GLA activity of Huh7 cell lines transduced with AAV vectors comprising GLA operably linked to one of the HCR-hAAT, NP1, NP2, NP5 or NP6 TREs. Cells were transduced with the vectors and GLA activity measured in culture supernatants via 4-MUG assay three days post transduction.

FIG. 10—Graph showing the fold change in GLA activity of Huh7 cell lines transduced with AAV vectors comprising GLA operably linked to one of the HCR-hAAT, NP1, NP2, NP5 or NP6 TREs compared with HCR-hAAT. Cells were transduced with the vectors and GLA activity measured in culture supernatants via 4-MUG assay three days post transduction.

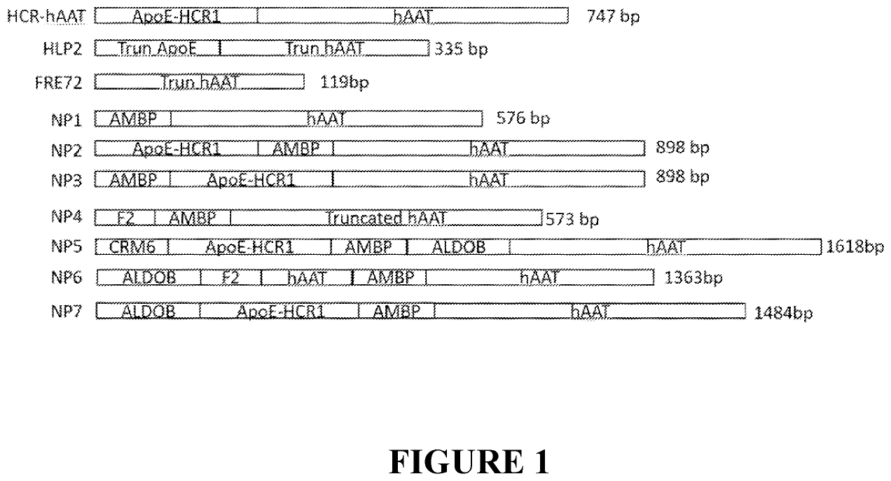

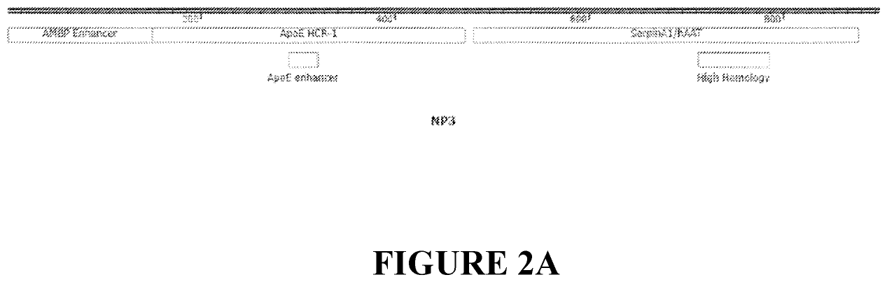

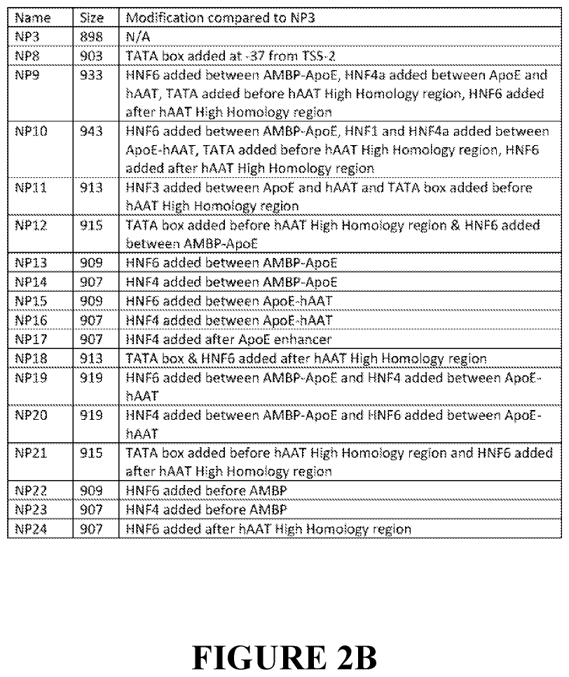

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a TRE comprising a hAAT sequence element” should be interpreted to mean that the TRE has a hAAT sequence element, but the TRE may comprise further sequence elements. Similarly, the phrase “a hAAT sequence element comprising a nucleotide sequence of SEQ ID NO: 30” refers to a sequence element that has a nucleotide sequence of SEQ ID NO: 30, but the sequence element may contain additional nucleotides.

In some embodiments of the invention, the word “comprising” is replaced with the phrase “consisting of”. The term “consisting of” is intended to be limiting. For example, the phrase “a TRE consisting of a nucleotide sequence of SEQ ID NO: 3” should be understood to mean that the TRE has a nucleotide sequence of SEQ ID NO: 3 and no additional nucleotides.

In some embodiments of the invention, the word “comprising” is replaced with the phrase “consisting essentially of”. The term “consisting essentially of” means that specific further components can be present, namely those not materially affecting the essential characteristics of the subject matter.

The terms “nucleic acid molecule” “nucleic acid sequence”, “polynucleotide” and “nucleotide sequence” are used interchangeably herein, and are intended to refer to a polymeric chain of nucleotides of any length e.g. deoxyribonucleotides, ribonucleotides, or analogs thereof. For example, the polynucleotide may comprise DNA (deoxyribonucleotides) or RNA (ribonucleotides). The polynucleotide may consist of DNA. The polynucleotide may be mRNA. Since the polynucleotide may comprise RNA or DNA, all references to T (thymine) nucleotides may be replaced with U (uracil).

The term “sequence element” is merely intended to refer to a polymeric chain of nucleotides of any length that is part of a longer chain of nucleotides. For example, a “hAAT sequence element” is a sequence element that is derived from a hAAT TRE and that forms part of a longer chain of nucleotides such as a TRE.

The term “equivalent expression” refers to an expression level that is the same as another expression level within a reasonable degree of scientific error. Optionally, expression levels are “equivalent” if they are within 10%, within 5%, within two standard deviations or within one standard deviation of one another. Similarly, a first TRE promotes equivalent expression than a second TRE if the level of expression promoted by the first TRE is within 10%, within 5%, within two standard deviations or within one standard deviation of the expression level of the second TRE. Expression levels may be determined using the “expression assay” discussed below.

The term “greater expression” refers to an expression level that is higher than another expression level. Optionally, the expression level is statistically significantly higher than the other expression level. Optionally, a first TRE promotes greater expression than a second TRE if the level of expression promoted by the first TRE is higher, optionally one standard deviation higher, than the expression level of the second TRE. For the avoidance of doubt, the terms “greater expression” and “higher expression” are considered to be interchangeable herein. Expression levels may be determined using the “expression assay” discussed below.

The term “corresponding TRE” refers to a TRE that is identical to a different TRE, but for one feature. For example, a “corresponding TRE lacking an AMBP sequence element” is a TRE which is identical to the TRE comprising an AMBP sequence element, except that it lacks an AMBP sequence element.

It is standard in the art that nucleotide sequences are written 5′ to 3′, i.e. the first nucleotide in any given sequence can be considered to be at the 5′ end and the last nucleotide can be considered to be at the 3′ end of any given nucleotide. Therefore, a sequence element that is 5′ of a second sequence element comes before the second sequence element in a nucleotide sequence (for example the nucleotide sequence of a TRE). A first sequence element that is 5′ of a second sequence element may come immediately before the second sequence element in the nucleotide sequence. Alternatively, a first sequence element that is 5′ of a second sequence element may not come immediately before the second sequence element in the nucleotide sequence, i.e. the nucleotide sequence may comprise an intervening sequence between the first and second sequence elements. Similarly, a first sequence element is less than 10 nucleotides 5′ of a second sequence element if the intervening sequence is less than 10 nucleotides in length.

A first sequence element that is 3′ of a second sequence element comes after the second sequence element in the nucleotide sequence. A first sequence element that is 3′ of a second sequence element may come immediately after the second sequence element in the nucleotide sequence, i.e. there are no intervening nucleotides between the two sequence elements. Alternatively, a first sequence element that is 3′ of a second sequence element may not come immediately after the second sequence element in the nucleotide sequence, i.e. the nucleotide sequence may comprise an intervening sequence between the first and second sequence elements. Similarly, a first sequence element is less than 10 nucleotides 3′ of a second sequence element if the intervening sequence is less than 10 nucleotides in length.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide or amino acid residues at each position are then compared. When a position in the first sequence is occupied by the same amino acid as the corresponding position in the second sequence, then the amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 80% identical to SEQ ID NO: 1, SEQ ID NO: 1 would be the reference sequence. To assess whether a sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

For the purposes of the present invention, the term “fragment” refers to a contiguous portion of a sequence. For example, a fragment of SEQ ID NO: 1 of 50 amino acids refers to 50 contiguous nucleotides of SEQ ID NO: 1.

The term “around” used in the context of describing the length of nucleotide or amino acid sequences indicates that a sequence may comprise or consist of a defined number of nucleotides or amino acids, plus or minus 10%, more particularly plus or minus 5%, or more particularly, plus or minus a single integer. For example, reference to an amino acid sequence of “around” 45 amino acids in length may refers to an amino acid sequence of 41-49 amino acids, more particularly 43-47 amino acids, and more particularly 44-46 amino acids in length.

The singular forms “a”. “an” and “the” include plural referents unless the content clearly dictates otherwise. Thus, for example, reference to “an amino acid” includes two or more instances or versions of such amino acids.

All publications, patents and patent applications cited herein, whether supra or infra, are hereby incorporated by reference in their entirety.

### Transcription Regulatory Element

In one aspect, the present invention provides a transcription regulatory element (TRE).

The term “transcription regulatory element” refers to a polynucleotide which can regulate the transcription of a gene to which it is operably linked.

A TRE may comprise one or more promoter and/or enhancer elements, such as a promoter and/or enhancer element selected from the group consisting of a hAAT sequence element, an AMBP sequence element, an ApoE-HCR1 sequence element, an ALDOB sequence element, a CRM6 sequence element, and an F2 sequence element. For example, a polynucleotide which comprises a hAAT sequence element is a TRE.

In one aspect, the TRE is greater than 500, greater than 600, greater than 700, greater than 800, greater than 900, greater than 1000, greater than 1100, greater than 1200, greater than 1300, greater than 1400, greater than 1500, or greater than 1600 nucleotides in length. In an aspect, the TRE is less than 2000, less than 1800, less than 1600, less than 1500, less than 1400, less than 1300, less than 1200, less than 1100, less than 1000, less than 900, less than 800, less than 700, or less than 600 nucleotides in length. In an aspect, the TRE is between 400 and 2000, between 500 and 1800, between 600 and 1400, between 800 and 1600, between 500 and 600, between 600 and 700, between 700 and 800, between 800 and 900, between 900 and 1000, between 1000 and 1100, between 1100 and 1200, between 1200 and 1300, between 1300 and 1400, between 1400 and 1500, between 1500 and 1600, or between 1600 and 1700 nucleotides in length.

In one embodiment, the TRE is between 563 and 1628 nucleotides in length. In one embodiment the TRE is between 563 and 583 nucleotides in length. In one embodiment, the TRE is between 566 and 586 nucleotides in length. In one embodiment, the TRE is between 888 and 908 nucleotides in length. In one embodiment, the TRE is between 893 and 913 nucleotides in length. In one embodiment, the TRE is between 897 and 917 nucleotides in length. In one embodiment, the TRE is between 899 and 919 nucleotides in length. In one embodiment, the TRE is between 903 and 923 nucleotides in length. In one embodiment, the TRE is between 905 and 925 nucleotides in length. In one embodiment, the TRE is between 909 and 929 nucleotides in length. In one embodiment, the TRE is between 913 and 933 nucleotides in length. In one embodiment, the TRE is between 923 and 943 nucleotides in length. In one embodiment, the TRE is between 933 and 953 nucleotides in length. In one embodiment, the TRE is between 1353 and 1373 nucleotides in length. In one embodiment, the TRE is between 1608 and 1628 nucleotides in length. In one embodiment, the TRE is between 1474 and 1494 nucleotides in length.

In one embodiment, the TRE is longer than FRE72. In one embodiment, the TRE is at least 450, at least 770, at least 1240, at least 1340, or at least 1390 nucleotides longer than FRE72. In one embodiment, the TRE is shorter than HCR-hAAT. In one embodiment, the TRE is at least 160 nucleotides shorter than HCR-hAAT. In one embodiment, the TRE is longer than HCR-hAAT. In one embodiment, the TRE is at least 150, at least 620, at least 720, or at least 770 nucleotides longer than HCR-hAAT. In one embodiment, the TRE is longer than FRE72 and shorter than HCR-hAAT. In one embodiment, the TRE is longer than 898 nucleotides in length. In an embodiment, the TRE is longer than 1363 nucleotides in length.

When determining the length of a TRE, the skilled person would understand that all nucleotides that are considered to be part of a TRE should be included (for example all nucleotide corresponding to a hAAT sequence element, an AMBP sequence element, an ApoE-HCR1, an ALDOB sequence element, a CRM6 sequence element, or an F2 sequence element should be included, and so should nucleotides corresponding to any other known transcription regulatory elements). For example, if a construct comprises two copies of the same TRE, then both copies should be included when the length is calculated.

In particular, when determining the length of a TRE, the nucleotides of any contiguous portion of a polynucleotide must be counted towards the length of the TRE if the contiguous portion comprises a nucleotide sequence having:
(a) at least 95% sequence identity to any of SEQ ID NO: 1-40;
(b) at least 95% sequence identity to any known promoter or enhancer element or fragment of any known promoter or enhancer element; and/or
(c) at least 95% sequence identity to any known transcription factor binding site.

Optionally, the TRE is comprised within a contiguous portion of less than 3000, less than 2500, less than 2000, or less than 1800 base pairs.

In one aspect, the TRE comprises or consists of:
(a) a nucleotide sequence having at least 80%, at least 85%, at least 90%, at least 95%, at least 98%, or at least 99% sequence identity to any one of SEQ ID NO: 3-26; or
(b) a nucleotide sequence of any one of SEQ ID NO: 3-26.

In one aspect, the TRE comprises or consists of:
(a) a nucleotide sequence having at least 80%, at least 85%, at least 90%, at least 95%, at least 98%, or at least 99% sequence identity to SEQ ID NO: 5; or
(b) a nucleotide sequence of SEQ ID NO: 5.

In one aspect, the TRE comprises or consists of:
(a) a nucleotide sequence having at least 80%, at least 85%, at least 90%, at least 95%, at least 98%, or at least 99% sequence identity to SEQ ID NO: 9; or
(b) a nucleotide sequence of SEQ ID NO: 9.

In one embodiment, the TRE comprises or consists of a nucleotide sequence having at least 95% sequence identity to SEQ ID NO: 3-26. In one embodiment, the TRE comprises or consists of a nucleotide sequence having at least 95% sequence identity to SEQ ID NO: 5. In one embodiment, the TRE comprises or consists of a nucleotide sequence having at least 95% sequence identity to SEQ ID NO: 9.

In one embodiment, the TRE comprises or consists of a nucleotide sequence having at least 98% sequence identity to SEQ ID NO: 3-26, In one embodiment, the TRE comprises or consists of a nucleotide sequence having at least 98% sequence identity to SEQ ID NO: 5.

In one embodiment, the TRE comprises or consists of a nucleotide sequence having at least 98% sequence identity to SEQ ID NO: 9.

Promotes Greater Expression than HCR-hAAT or FRE72

HCR-hAAT is a hybrid TRE derived from the human alpha-1-antitrypsin promoter and the enhancer/hepatic locus control region (HCR) of the ApoE gene. FRE72 is a TRE that comprises only the core region of the hAAT promoter. HCR-hAAT and FRE72 have been shown to promote high levels of expression of transgenes in human hepatocytes which have been transduced with a viral vector comprising an expression cassette comprising HCR-hAAT or FRE72 operably linked to said transgene.

In one aspect, the TRE promotes greater expression than HCR-hAAT. HCR-hAAT is a TRE that is described in more detail in Miao et al., Mol Ther. 2000; 1(6): 522-532, and has a sequence of SEQ ID NO: 2. The TRE may promote expression that is at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3 fold higher compared to HCR-hAAT. In one embodiment, the TRE promotes expression at least 1.8 fold higher compared to HCR-hAAT.

In an aspect, the TRE promotes expression that is between 1.2 fold and 3 fold, between 1.5 fold and 3 fold, between 1.8 fold and 3 fold, between 2 fold and 3 fold, between 2.5 fold and 3 fold, between 1.2 fold and 5 fold, between 1.5 fold and 5 fold, between 1.8 fold and 5 fold, between 2 fold and 5 fold, between 2.5 fold and 5 fold, or between 3 fold and 5 fold higher compared to HCR-hAAT. In one embodiment, the TRE promotes expression between 2 and 3 fold higher compared to HCR-hAAT.

In an aspect, the TRE promotes greater expression than FRE72. FRE72 is a promoter that is described in more detail in WO2021/084277 and has a sequence of SEQ ID NO: 1. The TRE may promote expression that is at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3 fold higher compared to FRE72. In one embodiment, the TRE promotes expression at least 1.8 fold higher compared to FRE72.

In an aspect, the TRE promotes expression that is between 1.2 fold and 3 fold, between 1.5 fold and 3 fold, between 1.8 fold and 3 fold, between 2 fold and 3 fold, between 2.5 fold and 3 fold, between 1.2 fold and 5 fold, between 1.5 fold and 5 fold, between 1.8 fold and 5 fold, between 2 fold and 5 fold, between 2.5 fold and 5 fold, or between 3 fold and 5 fold higher compared to FRE72. In one embodiment, the TRE promotes expression between 2 and 3 fold higher compared to FRE72.

### Expression Assay

The skilled person will understand that there are many suitable methods for determining the level of expression promoted by a TRE. A suitable expression assay is described in Example 1 and below.

Optionally, expression is measured by an expression assay comprising:
(i) transfecting human hepatocytes with a plasmid comprising an expression cassette comprising a transgene operably linked to the TRE;
(ii) incubating the transfected cells under conditions suitable for transgene expression to occur; and
(iii) measuring the level of the transgene by ELISA using an antibody specific for a protein encoded by the transgene.

Optionally, the transgene encodes a wild-type human CFI polypeptide. Optionally, transfection step (i) comprises mixing the plasmid with a transfection reagent (such as FUGENERHD (Promega, cat no. E2311) to form a transfection mixture, and delivering the transfection mixture to the hepatocytes. Optionally, the hepatocytes are Huh7 cells. The step of transfecting human hepatocytes may be a step of incubating the human hepatocytes with the transfection mixture overnight. The step of incubating the transfected cells may be a step of growing the transfected cells in suitable growth conditions for 5 days. The step of measuring the level of the transgene may comprise harvesting cells and media produced by the step of incubating the transfected cells after the step of incubating the transfected cells.

Optionally, transfection step (i) comprises co-transfecting the human hepatocytes with a plasmid comprising an expression cassette comprising a transgene encoding luciferase operably linked to a promoter and measuring total luciferase expression.

The step of measuring the level of the transgene by ELISA may be performed using a sandwich ELISA using two antibodies that bind to CFI (for example, using the CFI ELISA kit (e.g. Hycult cat #HK355-01) according to the manufacturer's instructions). For example, a first antibody that binds to CFI may be immobilised to a plate, and a portion of the cells and media that were optionally harvested as part of the step of measuring the level of the transgene may be applied to the plate with the first antibody immobilised on it. The plate may be washed, and then a second antibody that binds to CFI and is conjugated to a label may be applied to the plate. The second antibody may be conjugated to biotin, which binds to streptavidin-peroxidase, and in this case the streptavidin-peroxidase acts as the label. The plate may be washed again, and the amount of CFI bound to the plate measured by assessing the amount of the label present. For example, if the label is streptavidin-peroxidase, the substrate tetramethylbenzidine (TMB) may be added, which the streptavidin-peroxidase reacts with. The enzyme reaction may be stopped by adding oxalic acid. For each of the samples and standards, the absorbance at 450 nm may be measured using a spectrophotometer. A standard curve may be obtained by plotting the absorbance (linear) against the corresponding concentrations of human CFI standards. The concentration of human CFI in the samples may then be determined from the standard curve.

The measured expression may be compared to the expression measured when a different (reference) TRE is used, for example HCR-hAAT or FRE72. In such cases the above steps are repeated using the reference TRE in place of the TRE of the invention (the ‘test” TRE) (i.e. the user repeats the same steps, but inserts the reference TRE into the plasmid used in transfection step (i) in place of the test TRE).

As mentioned above, transfection step (i) may comprise co-transfecting the human hepatocytes with a plasmid comprising an expression cassette comprising a transgene encoding luciferase operably linked to a promoter and measuring the total luciferase expression. The plasmid comprising an expression cassette comprising a transgene encoding luciferase may be identical to the plasmid comprising an expression cassette comprising a transgene operably linked to the TRE (except that the transgenes are different and the TREs may be different). This allows the users to normalise the measured expression to reflect the number of cells that have been transfected (thereby accounting for differences in transfection efficiency), as the proportion of cells that are transfected by the vector (i.e. the transfection efficiency) will be proportionate to the total luciferase expression. In such cases, the expression assay further comprises a step of detecting the total luciferase expression. Optionally, the luciferase is operably linked to a CMV promoter.

The total luciferase expression may be measured by performing a luciferase assay on a portion of the cells that were optionally harvested as part of the step of measuring the level of transgene. The total luciferase expression may be measured by measuring luminescence using a luminometer. For example, the cells may be washed with phosphate buffered saline (PBS) twice and then treated with 100 μl of luciferase lysis buffer from the Luciferase assay kit (Promega cat #E1501/4530). Luciferase expression may be measured by measuring luminescence on a Molecular Devices SpextraMax i3x plate reader.

Optionally, the expression assay comprises calculating a normalised value for expression by dividing the level of the transgene measured by ELISA by the total luciferase expression from the corresponding cells.

Optionally, expression is measured by an activity assay comprising:
(i) transducing human hepatocytes with an AAV vector comprising an expression cassette comprising a transgene operably linked to the TRE:
(ii) incubating the transduced cells in conditions suitable for transgene expression to occur; and
(iii) measuring the level of the transgene by its activity.

Optionally, the transgene encodes a human alpha-galactosidase (GLA) polypeptide. Optionally, transduction step (i) comprises treating the hepatocytes with Mitomycin C. Optionally, the hepatocytes are Huh7 cells. The step of transducing human hepatocytes may be a step of incubating the human hepatocytes with the transduction mixture overnight. The step of incubating the transduced cells may be a step of growing the transduced cells in suitable growth conditions for 3 days.

The step of measuring the level of the transgene may be performed on the cell culture supernatant of the transduced cells. The step of measuring the level of the transgene may be performed using a GLA activity assay (4-MUG assay). For example, the cell supernatant may be incubated with a GLA activity assay reaction mix. The reaction mix may include 4-methylumbelliferyl-a-D-galactopyranoside. The reaction mix may also include N-acetyl-D-galactosamine. The reaction may be stopped after incubation, e.g. after incubation for 2 hours, by addition of glycine buffer. The activity of GLA may be quantified by measuring the level of 4-methylumbelliferone (4-MUG) by measuring fluorescence on a Molecular Devices Spectramax i3x plate reader at 365 nm excitation and emission at 450 nm. The activity of GLA may be measured by comparing the fluorescence of the sample to the fluorescence of known standards and blanks.

### Liver-Specific TRE

Optionally, the TRE is liver-specific. A TRE is “liver-specific” if it drives a higher level of expression in liver cells compared to other cells in general. For example, the skilled person can determine whether a TRE is a liver-specific TRE by comparing expression of the polynucleotide in liver cells (such as Huh 7 cells) with expression of the polynucleotide in cells from other tissues (such as kidney cells, for example HEK293T cells). If the level of expression is higher in the liver cells, compared to the cells from other tissues, the TRE is a liver-specific TRE. Optionally, a liver-specific TRE does not drive an appreciable level of expression in non-liver cells.

The TRE may promote gene expression in cells from at least one other organ or tissue at a level less than 40%, less than 30%, less than 25%, less than 15%, less than 10%, or less than 5% of the level that the TRE promotes gene expression in liver cells. Optionally, the TRE promotes gene expression in cells from at least one other organ or tissue at a level less than 5% of the level that the TRE promotes gene expression in liver cells. Optionally, the cells from at least one other organ or tissue are cells from one or more, two or more, three or more, four or more, or five or more of kidney cells, pancreatic cells, breast cells, neuroblastoma cells, lung cells, cardiomyocyte cells, and early B cells. Optionally, the cells from at least one other organ or tissue are cells from of kidney cells, pancreatic cells, breast cells, neuroblastoma cells, lung cells, cardiomyocyte cells, and early B cells. Optionally, the cells from at least one other organ or tissue are:
i) HEK293T cells:
(ii) 697 cells:
(iii) BxPC-3 cells:
(iv) MCF7 cells;
(v) 1643 cells:
(vi) MRC-9 cells; and/or
(vii) AC-16 cells.

Optionally, the liver cells are Huh7 cells. Optionally, the TRE promotes gene expression in HEK293T, 697, BxPC-3, MCF7, 1643, MRC-9 and AC-16 cells at a level less than 40%, less than 30%, less than 25%, less than 15%, less than 10%, or less than 5% of the level that the TRE promotes gene expression in Huh7 cells. Optionally, the TRE promotes gene expression in HEK293T, 697, BxPC-3, MCF7, 1643, MRC-9 and AC-16 cells at a level less than 5% of the level that the TRE promotes gene expression in Huh7 cells.

Whether or not a TRE promotes expression in particular (test) cells (like cells from at least one other organ or tissue) at a level less than the level that the TRE promotes gene expression in liver cells may be determined using a “tissue fidelity assay”. In a suitable tissue fidelity assay the user transduces liver cells and comparator cells with a vector comprising the TRE operably linked to a transgene. For example, if the user wishes to determine whether the TRE promotes higher gene expression in Huh7 cells compared to cells from at least one other organ or tissue then the liver cells used should be Huh7 cells. Similarly, if the user wishes to determine whether the TRE promoters higher gene expression in Huh7 cells compared to HEK293T kidney cells, the comparator cells used should be HEK293T cells. The user then measures whether the number of liver cells that express the transgene is higher than the number of comparator cells. For example, the transgene may be GFP, and the user may determine the number of cells (liver cells or comparator cells) that are transduced by counting the number of cells that fluoresce green (for example using FACS) or by measuring the mean fluorescence intensity. Suitable “tissue fidelity assays” are described in Example 2.

hAAT Sequence Element

In one aspect, the present invention provides a TRE that comprises a hAAT sequence element. A hAAT sequence element is a polynucleotide derived from the human alpha-1-antitrypsin promoter (hAAT promoter). An exemplary sequence of the hAAT promoter is given as SEQ ID NO: 29. The TRE may comprise three or fewer, two or fewer, or no more than one hAAT sequence element(s).

In one embodiment, the hAAT sequence element is less than 900, less than 800, less than 700, less than 600, less than 500, or less than 450 nucleotides in length.

In one embodiment, the TRE comprises a hAAT sequence element wherein the hAAT sequence element comprises a nucleotide sequence of SEQ ID NO: 30 or a variant of SEQ ID NO: 30 that differs by 1, 2, or 3 nucleotides. In one embodiment, the TRE comprises a hAAT sequence element wherein the hAAT sequence element comprises a nucleotide sequence of SEQ ID NO: 30. In one embodiment, the TRE comprises a hAAT sequence element wherein the hAAT sequence element comprises a nucleotide sequence of SEQ ID NO: 31 or a variant of SEQ ID NO: 31 that differs by 1, 2, or 3 nucleotides. In one embodiment, the TRE comprises a hAAT sequence element wherein the hAAT sequence element comprises a nucleotide sequence of SEQ ID NO: 31.

In one embodiment, the TRE comprises a hAAT sequence element wherein the hAAT sequence element comprises a nucleotide sequence at least 80%, at least 85%, at least 90%, at least 95%, at least 98%, or at least 99% identical to SEQ ID NO: 29 or a fragment of SEQ ID NO: 29 that is at least 200, at least 300, or at least 400 nucleotides long. In one embodiment, the TRE comprises a hAAT sequence element wherein the hAAT sequence element comprises a nucleotide sequence at least 98% identical to SEQ ID NO: 29. In one embodiment, the TRE comprises a hAAT sequence element wherein the hAAT sequence element comprises a nucleotide sequence of SEQ ID NO: 29. In one embodiment, the TRE comprises a second hAAT sequence element.

In one embodiment, the TRE comprises a first and a second hAAT sequence element, wherein the second hAAT sequence element is 5′ of the first hAAT sequence element and 5′ of the AMBP sequence element. In one embodiment, the second hAAT sequence element comprises a nucleotide sequence of SEQ ID NO: 30 or a variant of SEQ ID NO: 30 that differs by 1, 2, or 3 nucleotides. In one embodiment, the second hAAT sequence element comprises a nucleotide sequence of SEQ ID NO: 30. In embodiment, the first hAAT sequence element comprises SEQ ID NO: 29 and the second hAAT sequence element comprises SEQ ID NO: 30.

### AMBP Sequence Element

In one aspect, the present invention provides a TRE which comprises an AMBP sequence element. An AMBP sequence element is a polynucleotide derived from the human alpha1-microglobulin/bikunin enhancer (AMBP enhancer). The AMBP enhancer is further described in Rouet et al., Journal of Biological Chemistry, 1992, 267: 20765-20773. An exemplary sequence for the AMBP enhancer is given as SEQ ID NO: 32. The TRE may comprise three or fewer, two or fewer, or no more than one AMBP sequence element(s).

In one embodiment, the TRE comprises an AMBP sequence element wherein the AMBP sequence element comprises a nucleotide sequence of SEQ ID NO: 33 or a variant of SEQ ID NO: 33 that differs by 1, 2, or 3 nucleotides. In one embodiment, the TRE comprises an AMBP sequence element wherein the AMBP sequence element comprises a nucleotide sequence of SEQ ID NO: 33. In one embodiment, the TRE comprises an AMBP sequence element wherein the AMBP sequence element comprises a nucleotide sequence of SEQ ID NO: 32 or a variant of SEQ ID NO: 32 that differs by 1, 2, or 3 nucleotides. In one embodiment, the TRE comprises an AMBP sequence element wherein the AMBP sequence element comprises a nucleotide sequence of SEQ ID NO: 32.

An AMBP sequence element of the present invention may comprise binding sites for one or more transcription factors. Accordingly, in one aspect, the AMBP sequence element comprises at least one binding site selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises at least two binding sites selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises at least three binding sites selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises at least four binding sites selected from the group consisting of an HNF1-1 binding site, an HNF4 binding site, an HNF3a binding site, an HNF1-2 binding site, and an HNF3-2 binding site. In an aspect, the TRE comprises an AMBP sequence element, wherein the AMBP sequence element comprises an HNF1-1 binding site, an HNF4 binding site, an HNF3a binding site, an HNF1-2 binding site, and an HNF3-2 binding site.

In one aspect, the AMBP sequence element is 5′ of a hAAT sequence element. Optionally, the AMBP sequence element is less than 10, less than 8, or less than 5 nucleotides 5′ of the hAAT sequence element. Optionally, the AMBP sequence element is immediately 5′ of the hAAT sequence element.

In one aspect, a TRE comprising the AMBP sequence element promotes equivalent or greater expression than a corresponding TRE lacking an AMBP sequence element. The skilled person can determine whether a TRE comprising an AMBP sequence element (a test TRE) promotes equivalent or greater expression than a second TRE not comprising an AMBP sequence element (a reference TRE) by determining the level of expression promoted by the test TRE comprising an AMBP sequence element and the reference TRE using the techniques described under the heading “expression assay”. Optionally, the user determines whether a TRE comprising an AMBP sequence element (the test AMBP sequence element) promotes equivalent or greater expression than a reference TRE not comprising an AMBP sequence element by creating a test TRE that comprises the test AMBP sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an AMBP sequence element.

### ApoE-HCR1 Sequence Element

In one aspect, the present invention provides a TRE which comprises an ApoE-HCR1 sequence element. An ApoE-HCR1 sequence element is a polynucleotide derived from the hepatic locus control region (HCR) of the ApoE gene. The ApoE-HCR1 is described further in Miao et al (2000), Molecular Therapy 1(6):522. An exemplary sequence for the ApoE-HCR1 is given as SEQ ID NO: 27. The TRE may comprise three or fewer, two or fewer, or no more than one ApoE-HCR1 sequence element(s).

In one embodiment, the ApoE-HCR1 sequence element is less than 800, less than 700, less than 600, less than 500, less than 400, or less than 350 nucleotides in length. In one embodiment, the ApoE-HCR1 sequence element is greater than 50, greater than 100, greater than 200, or greater than 300 nucleotides in length. In one embodiment, the ApoE-HCR1 sequence element is between 50 and 350, between 100 and 350, between 200 and 350 or between 300 and 350 nucleotides in length.

In one embodiment, the TRE comprises an ApoE-HCR1 sequence element wherein the ApoE-HCR1 sequence element comprises a nucleotide sequence of SEQ ID NO: 28 or a variant of SEQ ID NO: 28 that differs by 1, 2, or 3 nucleotides. In one embodiment, the TRE comprises an ApoE-HCR1 sequence element wherein the ApoE-HCR1 sequence element comprises a nucleotide sequence of SEQ ID NO: 28. In one embodiment, the TRE comprises an ApoE-HCR1 sequence element wherein the ApoE-HCR1 sequence element comprises a nucleotide sequence of SEQ ID NO: 27 or a variant of SEQ ID NO: 27 that differs by 1, 2, or 3 nucleotides. In one embodiment, the TRE comprises an ApoE-HCR1 sequence element wherein the ApoE-HCR1 sequence element comprises a nucleotide sequence of SEQ ID NO: 27.

In one aspect, the ApoE-HCR1 sequence element is 5′ of a hAAT sequence element. Optionally, the ApoE-HCR1 sequence element is less than 10, less than 8, or less than 5 nucleotides 5′ of the hAAT sequence element. Optionally, the ApoE-HCR1 sequence element is immediately 5′ of the hAAT sequence element. In one aspect, the ApoE-HCR1 sequence element is 5′ of an AMBP sequence element. Optionally, the ApoE-HCR1 sequence element is less than 10, less than 8, or less than 5 nucleotides 5′ of the AMBP sequence element. Optionally, the ApoE-HCR1 sequence element is immediately 5′ of the AMBP sequence element. In an aspect, the ApoE-HCR1 sequence element is 3′ of an AMBP sequence element. Optionally, the ApoE-HCR1 sequence element is less than 10, less than 8, or less than 5 nucleotides 5′ of the AMBP sequence element. Optionally, the ApoE-HCR1 sequence element is immediately 5′ of the AMBP sequence element. In an aspect, the ApoE-HCR1 sequence element is 5′ of both an hAAT sequence element and an AMBP sequence element. In an aspect, the ApoE-HCR1 sequence element is 5′ of a hAAT sequence element and 3′ of an AMBP sequence element.

In one aspect, a TRE comprising the ApoE-HCR1 sequence element promotes equivalent or greater expression than a corresponding TRE lacking an ApoE-HCR1 sequence element. The skilled person can determine whether a TRE comprising an ApoE-HCR1 sequence element (a test TRE) promotes equivalent or greater expression than a second TRE not comprising an ApoE-HCR1 sequence element (a reference TRE) by determining the level of expression promoted by the test TRE comprising an ApoE-HCR1 and the reference TRE using the techniques described under the heading “expression assay”. Optionally, the user determines whether a TRE comprising an ApoE-HCR1 sequence element (the test ApoE-HCR1 sequence element) promotes equivalent or greater expression than a reference TRE not comprising an ApoE-HCR1 sequence element by creating a test TRE that comprises the test ApoE-HCR1 sequence element 5′ of hAAT and comparing the level of expression of the test TRE with that of hAAT (the reference TRE). If the level of expression of the test TRE is equivalent or higher than the level of expression of hAAT, then the TRE promotes equivalent or greater expression than a corresponding TRE not comprising an ApoE-HCR1 sequence element.

### CRM6 Sequence Element

In one aspect, the present invention provides a TRE comprising a CRM6 sequence element. A CRM6 sequence element is a polynucleotide derived from CRM6. CRM6 is described in further detail in Chuah et al., Molecular Therapy, 2014, 22: 1605-1613. An exemplary sequence for CRM6 is given as SEQ ID NO: 34. The TRE may comprise three or fewer, two or fewer, or no more than one CRM6 sequence element(s).

In one embodiment, the TRE comprises a CRM6 sequence element wherein the CRM6 sequence element comprises a nucleotide sequence of SEQ ID NO: 35 or a variant of SEQ ID NO: 35 that differs by 1, 2, or 3 nucleotides. In one embodiment, the TRE comprises a CRM6 sequence element wherein the CRM6 sequence element comprises a nucleotide sequence of SEQ ID NO: 35. In one embodiment, the TRE comprises a CRM6 sequence element wherein the CRM6 sequence element comprises a nucleotide sequence of SEQ ID NO: 36 or a variant of SEQ ID NO: 36 that differs by 1, 2, or 3 nucleotides. In one embodiment, the TRE comprises a CRM6 sequence element wherein the CRM6 sequence element comprises a nucleotide sequence of SEQ ID NO: 36. In one embodiment, the TRE comprises a CRM6 sequence element wherein the CRM6 sequence element comprises a nucleotide sequence of SEQ ID NO: 34 or a variant of SEQ ID NO: 34 that differs by 1, 2, or 3 nucleotides. In one embodiment, the TRE comprises a CRM6 sequence element wherein the CRM6 sequence element comprises a nucleotide sequence of SEQ ID NO: 34.

A CRM6 sequence element of the present invention may comprise binding sites for one or more transcription factors. Accordingly, in one aspect, the CRM6 sequence element comprises at least one binding site selected from the group consisting of an Sp1 binding site, an Sp2 binding site, an HNF3A binding site, and an HNF-1 binding site. In an aspect, the CRM6 sequence element comprises at least two binding sites selected from the group consisting of an Sp1 binding site, an Sp2 binding site, an HNF3A binding site, and an HNF-1 binding site. In an aspect the CRM6 sequence element comprises at least three binding sites selected from the group consisting of an Sp1 binding site, an Sp2 binding site, an HNF3A binding site, and an HNF-1 binding site. In an aspect, the present invention provides a TRE which comprises a CRM6 sequence element, wherein the CRM6 sequence element comprises an Sp1 binding site, an Sp2 binding site, an HNF3A binding site, and an HNF-1 binding site.

In one aspect, the CRM6 sequence element is 5′ of a hAAT sequence element. Optionally, the CRM6 sequence element is less than 10, less than 8, or less than 5 nucleotides 5′ of the hAAT sequence element. Optionally, the CRM6 sequence element is immediately 5′ of the hAAT sequence element. In one aspect, the CRM6 sequence element is 5′ of an AMBP sequence element. Optionally, the CRM6 sequence element is less than 10, less than 8, or less than 5 nucleotides 5′ of the AMBP sequence element. Optionally, the CRM6 sequence element is immediately 5′ of the AMBP sequence element. In an aspect, the CRM6 sequence element is 3′ of an AMBP sequence element. Optionally, the CRM6 sequence element is less than 10, less than 8, or less than 5 nucleotides 3′ of the AMBP sequence element. Optionally, the CRM6 sequence element is immediately 3′ of the AMBP sequence element. In an aspect, the CRM6 sequence element is 5′ of both an hAAT sequence element and an AMBP sequence element. In an aspect, the CRM6 sequence element is 5′ of a hAAT sequence element and 3′ of an AMBP sequence element.

In one aspect, a TRE comprising the CRM6 sequence element promotes equivalent or greater expression than a corresponding TRE lacking an CRM6 sequence element. The skilled person can determine whether a TRE comprising an CRM6 sequence element (a test TRE) promotes equivalent or greater expression than a second TRE not comprising an CRM6 sequence element (a reference TRE) by determining the level of expression promoted by the test TRE comprising a CRM6 sequence element and the reference TRE using the techniques described under the heading “expression assay”. Optionally, the user determines whether a TRE comprising an CRM6 sequence element (the test CRM6 sequence element) promotes equivalent or greater expression than a reference TRE not comprising an CRM6 sequence element by creating a test TRE that comprises the test CRM6 sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an CRM6 sequence element.

### ALDOB Sequence Element

In one aspect, the present invention provides a TRE comprising an ALDOB sequence element. An ALDOB sequence element is a polynucleotide derived from the enhancer of the aldolase B gene. The ALDOB enhancer is described in further detail in Gregori 2002 and Patwardhan et al. 2012. An exemplary sequence for ALDOB is given as SEQ ID NO: 37. The TRE may comprise three or fewer, two or fewer, or no more than one ALDOB sequence element(s).

In one embodiment, the TRE comprises an ALDOB sequence element wherein the ALDOB sequence element comprises a nucleotide sequence of SEQ ID NO: 38 or a variant of SEQ ID NO: 38 that differs by 1, 2, or 3 nucleotides. In one embodiment, the TRE comprises an ALDOB sequence element wherein the ALDOB sequence element comprises a nucleotide sequence of SEQ ID NO: 38. In one embodiment, the TRE comprises an ALDOB sequence element wherein the ALDOB sequence element comprises a nucleotide sequence of SEQ ID NO: 37 or a variant of SEQ ID NO: 37 that differs by 1, 2, or 3 nucleotides. In one embodiment, the TRE comprises an ALDOB sequence element wherein the ALDOB sequence element comprises a nucleotide sequence of SEQ ID NO: 37.

An ALDOB sequence element of the present invention may comprise binding sites for one or more transcription factors. Accordingly, in one aspect, the ALDOB sequence element comprises at least one binding site selected from the group consisting of an SP1 binding site, an HNF1-a binding site, a C/EBP binding site, a GATA2 binding site, a USF1 binding site, and a USF2 binding site. In an aspect, the ALDOB sequence element comprises at least two binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least three binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least four binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least five binding sites selected from the group consisting of an SP1 binding site, an HNF1-a binding site, a C/EBP binding site, a GATA2 binding site, a USF1 binding site, and a USF2 binding site. In an aspect, the ALDOB sequence element comprises at least one SP1 binding site, at least one HNF1-a binding site, at least one C/EBP binding site, at least one GATA2 binding site, at least one USF1 binding site, and at least one USF2 binding site.

In one aspect, the ALDOB sequence element is 5′ of a hAAT sequence element. Optionally, the ALDOB sequence element is less than 10, less than 8, or less than 5 nucleotides 5′ of the hAAT sequence element. Optionally, the ALDOB sequence element is directly 5′ of the hAAT sequence element. In one aspect, the ALDOB sequence element is 5′ of an AMBP sequence element. Optionally, the ALDOB sequence element is less than 10, less than 8, or less than 5 nucleotides 5′ of the AMBP sequence element. Optionally, the ALDOB sequence element is immediately 5′ of the AMBP sequence element. In an aspect, the ALDOB sequence element is 3′ of an AMBP sequence element. Optionally, the ALDOB sequence element is less than 10, less than 8, or less than 5 nucleotides 3′ of the AMBP sequence element. Optionally, the ALDOB sequence element is immediately 3′ of the AMBP sequence element. In an aspect, the ALDOB sequence element is 5′ of both an hAAT sequence element and an AMBP sequence element. In an aspect, the ALDOB sequence element is 5′ of a hAAT sequence element and 3′ of an AMBP sequence element.

In one aspect, a TRE comprising the ALDOB sequence element promotes equivalent or greater expression than a corresponding TRE lacking an ALDOB sequence element. The skilled person can determine whether a TRE comprising an ALDOB sequence element (a test TRE) promotes equivalent or greater expression than a second TRE not comprising an ALDOB sequence element (a reference TRE) by determining the level of expression promoted by the test TRE comprising an AMBP sequence and the reference TRE using the techniques described under the heading “expression assay”. Optionally, the user determines whether a TRE comprising an ALDOB sequence element (the test ALDOB sequence element) promotes equivalent or greater expression than a reference TRE not comprising an ALDOB sequence element by creating a test TRE that comprises the test ALDOB sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an ALDOB sequence element.

### F2 Sequence Element

In one aspect, the present invention provides a TRE comprising an F2 sequence element. An F2 sequence element is a polynucleotide derived from the enhancer of the prothrombin gene. An exemplary sequence for the prothrombin enhancer is given as SEQ ID NO: 39. The TRE may comprise three or fewer, two or fewer, or no more than one F2 sequence element(s).

In one embodiment, the TRE comprises an F2 sequence element wherein the F2 sequence element comprises a nucleotide sequence of SEQ ID NO: 40 or a variant of SEQ ID NO: 40 that differs by 1, 2, or 3 nucleotides. In one embodiment, the TRE comprises an F2 sequence element wherein the F2 sequence element comprises a nucleotide sequence of SEQ ID NO: 40. In one embodiment, the TRE comprises an F2 sequence element wherein the F2 sequence element comprises a nucleotide sequence of SEQ ID NO: 39 or a variant of SEQ ID NO: 39 that differs by 1, 2, or 3 nucleotides. In one embodiment, the TRE comprises an F2 sequence element wherein the F2 sequence element comprises a nucleotide sequence of SEQ ID NO: 39.

An F2 sequence element of the present invention may comprise binding sites for one or more transcription factors. Accordingly, in one aspect, the F2 sequence element comprises at least one binding site selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least two binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least three binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least four binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least five binding sites selected from the group consisting of HNF4a binding site, an HNF3a binding site, an HNF1a binding site, an HNF3b binding site, an STAT3 binding site, an RBPJ binding site, and an SP3 binding site. In an aspect, the F2 sequence element comprises at least six binding sites selected from the group consisting of HNF4a binding site, an HNF3a binding site, an HNF1a binding site, an HNF3b binding site, an STAT3 binding site, an RBPJ binding site, and an SP3 binding site. In an aspect, the TRE comprises an F2 sequence element, wherein the F2 sequence element comprises an HNF4a binding site, an HNF3a binding site, an HNF1a binding site, an HNF3b binding site, an STAT3 binding site, an RBPJ binding site, and an SP3 binding site.

In one aspect, the F2 sequence element is 5′ of a hAAT sequence element. Optionally, the F2 sequence element is less than 10, less than 8, or less than 5 nucleotides 5′ of the hAAT sequence element. Optionally, the F2 sequence element is immediately 5′ of the hAAT sequence element. In one aspect, the F2 sequence element is 5′ of an AMBP sequence element. Optionally, the F2 sequence element is less than 10, less than 8, or less than 5 nucleotides 5′ of the AMBP sequence element. Optionally, the F2 sequence element is immediately 5′ of the AMBP sequence element. In an aspect, the F2 sequence element is 3′ of an AMBP sequence element. Optionally, the F2 sequence element is less than 10, less than 8, or less than 5 nucleotides 3′ of the AMBP sequence element. Optionally, the F2 sequence element is immediately 5′ of the AMBP sequence element. In an aspect, the F2 sequence element is 5′ of both an hAAT sequence element and an AMBP sequence element. In an aspect, the F2 sequence element is 5′ of a hAAT sequence element and 3′ of an AMBP sequence element.

In one aspect, a TRE comprising the F2 sequence element promotes equivalent or greater expression than a corresponding TRE lacking an F2 sequence element. The skilled person can determine whether a TRE comprising an F2 sequence element (a test TRE) promotes equivalent or greater expression than a second TRE not comprising an F2 sequence element (a reference TRE) by determining the level of expression promoted by the test TRE comprising an F2 sequence element and the reference TRE using the techniques described under the heading “Expression assay”. Optionally, the user determines whether a TRE comprising an F2 sequence element (the test F2 sequence element) promotes equivalent or greater expression than a reference TRE not comprising an F2 sequence element by creating a test TRE that comprises the test F2 sequence element 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an F2 sequence element.

### Exemplary TRES

In one embodiment, the TRE (such as NP1) comprises:
(i) an AMBP sequence element; and
(ii) a hAAT sequence element,

wherein
(i) the AMBP sequence element comprises a nucleotide sequence of SEQ ID NO: 32;
(ii) the hAAT sequence element comprises a nucleotide sequence of SEQ ID NO: 29;
(iii) the AMBP sequence element is 5′ of the hAAT sequence element; and
(iv) the TRE is between 550 and 600 nucleotides in length.

In one embodiment, the TRE (such as NP2) comprises:
(i) an AMBP sequence element;
(ii) a hAAT sequence element; and
(iii) an ApoE-HCR1 sequence element,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of both the AMBP sequence element and the hAAT sequence element; and
(vi) the TRE is between 875 and 925 nucleotides in length.

In one embodiment, the TRE (such as NP3) comprises:
(i) an AMBP sequence element;
(ii) a hAAT sequence element; and
(iii) an ApoE-HCR1 sequence element,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element; and
(vi) the TRE is between 875 and 925 nucleotides in length.

In one embodiment, the TRE (such as NP4) comprises:
(i) an AMBP sequence element;
(ii) a hAAT sequence element; and
(iii) an F2 sequence element,
   wherein:
(i) the AMBP sequence element comprises a nucleotide sequence of SEQ ID NO: 32;
(ii) the hAAT sequence element comprises a fragment of SEQ ID NO: 29 that is between 300 and 350 nucleotides in length;
(iii) the hAAT sequence element comprises a nucleotide sequence of SEQ ID NO: 30;
(iv) the F2 sequence element comprises a nucleotide sequence of SEQ ID NO: 39;
(v) the AMBP sequence element is 5′ of the hAAT sequence element;
(vi) the F2 sequence element is 5′ of both the AMBP sequence element and the hAAT sequence element; and
(vii) the TRE is between 550 and 600 nucleotides in length.

In one embodiment, the TRE (such as NP5) comprises:
(i) an AMBP sequence element;
(ii) a hAAT sequence element;
(iii) an ApoE-HCR1 sequence element;
(iv) a CRM6 sequence element; and
(v) an ALDOB sequence element,

wherien
(i) the AMBP sequence element comprises a nucleotide sequence of SEQ ID NO: 32:
(ii) the hAAT sequence element comprises a nucleotide sequence of SEQ ID NO: 29;
(iii) the ApoE-HCR1 sequence element comprises a nucleotide sequence of SEQ ID NO: 27:
(iv) the CRM6 sequence element comprises a nucleotide sequence of SEQ ID NO: 34;
(v) the ALDOB sequence element comprises a nucleotide sequence of SEQ ID NO: 37:
(vi) the AMBP sequence element is 5′ of the hAAT sequence element;
(vii) the ApoE-HCR1 sequence element is 5′ of both the AMBP sequence element and the hAAT sequence element;
(viii) the CRM6 sequence element is 5′ of the AMBP sequence element, the hAAT sequence element, and the ApoE-HCR1 sequence element;
(ix) the ALDOB sequence element is 5′ of the hAAT sequence element and 3′ of the AMBP sequence element; and
(x) the TRE is between 1600 and 1650 nucleotides in length.

In one embodiment, the TRE (such as NP6) comprises:
(i) an AMBP sequence element;
(ii) a first hAAT sequence element:
(iii) a second hAAT sequence element:
(iv) an F2 sequence element; and
(v) an ALDOB sequence element,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the second hAAT sequence element comprises a nucleotide sequence of SEQ ID NO: 30;
(iv) the F2 sequence element comprises a nucleotide sequence of SEQ ID NO: 39;
(v) the ALDOB sequence element comprises a nucleotide sequence of SEQ ID NO: 37:
(vi) the AMBP sequence element is 5′ of the hAAT sequence element:
(vii) the second hAAT sequence element is 5′ of both the AMBP sequence element and the hAAT sequence element:
(viii) the F2 sequence element is 5′ of the AMBP sequence element, the hAAT sequence element, and the second hAAT sequence element:
(ix) the ALDOB sequence element is 5′ of the hAAT sequence element, the AMBP sequence element, the second hAAT sequence element, and the F2 sequence element; and
(x) the TRE is between 1350 and 1400 nucleotides in length.

In one embodiment, the TRE (such as NP7) comprises:
(i) an AMBP sequence element:
(ii) a hAAT sequence element:
(iii) an ApoE-HCR1 sequence element; and
(iv) an ALDOB sequence element, wherein:
(i) the AMBP sequence element comprises a nucleotide sequence of SEQ ID NO: 32:
(ii) the hAAT sequence element comprises a nucleotide sequence of SEQ ID NO: 29:
(iii) the ApoE-HCR1 sequence element comprises a nucleotide sequence of SEQ ID NO: 27:
(iv) the ALDOB sequence element comprises a nucleotide sequence of SEQ ID NO: 37:
(v) the AMBP sequence element is 5′ of the hAAT sequence element;
(vi) the ApoE-HCR1 sequence element is 5′ of both the AMBP sequence element and the hAAT sequence element:
(vii) the ALDOB sequence element is 5′ of the hAAT sequence element, the AMBP sequence element, and the ApoE-HCR1 sequence element; and
(viii) the TRE is between 1450 and 1500 nucleotides in length.

### Transcription Factor Binding Site

A transcription factor binding site is a nucleotide sequence to which a transcription factor can bind. Binding of a transcription factor to a transcription factor binding site may result in changes to the expression level of the polynucleotide comprises said binding site. Table 1 provides exemplary sequences for several transcription factor binding sites, but the skilled person will understand that this list is non-exhaustive and transcription factors may bind to alternative sites.

TABLE 1

| TF Binding Site | Sequence | SEQ ID NO |
|---|---|---|
| Alb | TAGACTAATATTTG | 43 |
| C/EBP | CTTGCAAAA | 44 |
| | TATGTACATT | 45 |
| | CCCACAACAT | 46 |
| | TTGAGAAAT | 47 |
| | GATTCATCTC | 48 |
| | TTGGTCTAAC | 49 |
| | CTTTCTACCTTG | 50 |
| | CTGTTTGCTC | 51 |
| C/EBP alpha | CTTGCAAAA | 52 |
| | CTGTTTGCTC | 53 |
| CTCT | CCCAGGTGT | 54 |
| CUX2 | TATTGATT | 55 |
| DUX4 | AAAAGCAGTCA | 56 |
| E2F6 | AGGCAGGAGAA | 57 |
| | GAGAAGAAATC | 58 |
| ELK4 | CAACATCCTGG | 59 |
| | ACTTATCCTCT | 60 |
| | CGTGTTCCTGC | 61 |
| FOX | GCAAACAGG | 62 |
| | TCATGTT | 63 |
| FOXD1 | ATCAACAT | 64 |
| GATA1 | ATAATCTCAGGAGCACAA | 65 |
| GATA2 | TTATCT | 66 |
| | TGATAG | 67 |
| | AGATAG | 68 |
| | GACTTATCCTC | 69 |
| HNF1 | TGTTTATTCAACACCTCT | 70 |
| | GTTAATAATC | 71 |
| HNF1a | GACTAATATTTGC | 72 |
| | CTTGCAAAATTAT | 73 |
| | GCTGCTAATTTTT | 74 |
| | ACAAATAATTAAC | 75 |
| HNF1-1 | GTTAATTTTTAAA | 76 |
| HNF1-2 | GGTTAATAATC | 77 |
| HNF1b | TGGTTAATATTC | 78 |
| HNF3 | TGTTTGCTCT | 79 |
| | AGCACAAACA | 80 |
| | TGTTTGCTC | 81 |
| HNF3a | TGTTTGC | 82 |
| | TGTTTGCT | 83 |
| | AGAAATCAACA | 84 |
| | CTAATATTTGCCTTG | 85 |
| HNF3b | GACTAATATTTGC | 86 |
| | TCTGTTTGCTCT | 87 |
| HNF4a | TTCCTGCTCTTTGTCCC | 88 |
| | CAGAGTTT | 89 |
| | CCCTGTTTGCTC | 90 |
| | TGGCCCTTGC | 91 |
| HNF4g | AGCAGTCAAAAGTCC | 92 |
| HNF6 | TTATTGATATTC | 93 |
| | TATTGATT | 94 |
| IRF | TTTTTTTTCCAATAT | 95 |
| | ATTTACTCTCTCTGTTTGCTC | 96 |
| | TTACTCTCTCTGTTTGCTCTG | 97 |
| JUN | TTAATAATCTCAG | 98 |
| LF-B1 | TGGTTAATATTCACCAG | 99 |
| KLF4 | CAACATCCTGG | 100 |
| MXL1 | AGTCCAAGTGGCC | 101 |
| MYC | GTCCAAGTGGCC | 102 |
| MZF1 | CTCTGGTTAA | 103 |
| | AAATGGGGGA | 104 |
| NF-KB | TCAAAAGTCC | 105 |
| NHF1 | TAGACTAATATTTG | 106 |
| NR2C2 | CAGGAGAAGAAATCA | 107 |
| Oct-1 | TGTTAATTAAAAGC | 108 |
| POU6F1 | GTTAATTTTT | 109 |
| PRDM1 | TTACTCTCTC | 110 |
| RBPJ | CTTGGGTACT | 111 |
| Serpina | GCAAACAGG | 112 |
| SHOX | TTAATTTT | 113 |
| SP1 | GGGATTCA | 114 |
| | GGTACTGT | 115 |
| | TTAATCCC | 116 |
| | GGTATTGA | 117 |
| | GGCTCTAA | 118 |
| | GGCATGAT | 119 |
| | TTCAAACC | 120 |
| | ATAGAACC | 121 |
| | TGGAGGCAGG | 122 |
| SPIB | ACAGGAA | 123 |
| SPL1/SP3 | ATGGGGGAGGGAC | 124 |
| SRY | GCAGTCAAA | 125 |
| | GAGCACAAA | 126 |
| STAT1 | ATTCCTGGAGG | 127 |
| STAT3 | CAAACAGGAAA | 128 |
| TATA Box | TATAAA | 129 |
| | TATATAAA | 130 |
| TEAD2 | AAACATTCCTGGA | 131 |
| USF1 | ACGTGTG | 132 |
| | CAGGTGTCA | 133 |
| | CAGATGTCA | 134 |
| | TCCAAGTGGCC | 135 |
| USF1/2 | AGCATGTGATA | 136 |

In an aspect, the TRE comprises one or more transcription factor binding sites. In an aspect, the TRE comprises
a) a TATA box;
b) an HNF6 binding site;
c) an HNF4a binding site;
d) an HNF1 binding site; and/or
e) an HNF3 binding site.

In an aspect, the TRE comprises a TATA box, optionally a canonical TATA box, optionally wherein the TRE comprises a hAAT sequence element and the TATA box is inserted into the hAAT sequence element.

A canonical TATA box is an AT-rich nucleotide sequence, capable of binding TATA-binding protein (TBP), which comprises the nucleotide sequence TATA, for example TATAAAA (SEQ ID NO: 137) is a canonical TATA box. A non-canonical TATA box is an AT-rich nucleotide sequence, capable of binding TATA-binding protein (TBP), which does not comprise the nucleotide sequence TATA, for example TTAAATA (SEQ ID NO: 138). A hAAT sequence element may comprise a non-canonical TATA box. A TRE of the present invention may comprise a hAAT sequence element, wherein the hAAT sequence element comprises a non-canonical TATA box.

The hAAT sequence element may comprise a nucleotide sequence of SEQ ID NO: 30 or a variant of SEQ ID NO: 30 that differs by 1, 2, or 3 nucleotides and the TATA box may be 3′ of the nucleotide sequence of SEQ ID NO: 30 or variant of SEQ ID NO: 30. The hAAT sequence element may comprise a nucleotide sequence of SEQ ID NO: 30 or a variant of SEQ ID NO: 30 that differs by 1, 2, or 3 nucleotides and the TATA box may be 5′ of the nucleotide sequence of SEQ ID NO: 30 or variant of SEQ ID NO: 30.

The TRE may promote equivalent or greater expression than a corresponding TRE not comprising the TATA box.

In one aspect, a TRE comprising the TATA box promotes equivalent or greater expression than a corresponding TRE lacking a TATA box. The skilled person can determine whether a TRE comprising a TATA box (a test TRE) promotes equivalent or greater expression than a second TRE not comprising a TATA box (a reference TRE) by determining the level of expression promoted by the test TRE comprising a TATA box and the reference TRE using the techniques described under the heading “expression assay”. Optionally, the user determines whether a TRE comprising a TATA box (the test TATA box) promotes equivalent or greater expression than a reference TRE not comprising a TATA box by creating a test TRE that comprises the test TATA box 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising a TATA box.

In an aspect, the TRE comprises an HNF6 binding site. Optionally, the HNF6 binding site has a nucleotide sequence of SEQ ID NO: 93 or 94 as set out above.

In an aspect, the TRE comprises an HNF6 binding site, and:
a. the TRE comprises a hAAT sequence element that comprises a nucleotide sequence of SEQ ID NO: 30 or a variant of SEQ ID NO: 30 that differs by 1, 2, or 3 nucleotides and the HNF6 binding site is 5′ of the hAAT sequence element that comprises a nucleotide sequence of SEQ ID NO: 30 or a variant of SEQ ID NO: 30 that differs by 1, 2, or 3 nucleotides; and/or
b. the TRE comprises a hAAT sequence element that comprises a nucleotide sequence of SEQ ID NO: 31 or a variant of SEQ ID NO: 31 that differs by 1, 2, or 3 nucleotides and the HNF6 binding site is 5′ of the hAAT sequence element that comprises a nucleotide sequence of SEQ ID NO: 31 or a variant of SEQ ID NO: 31 that differs by 1, 2, or 3 nucleotides; and/or
c. the TRE comprises a hAAT sequence element that comprises a nucleotide sequence of SEQ ID NO: 29 or a variant of SEQ ID NO: 29 that differs by 1, 2, or 3 nucleotides and the HNF6 binding site is 5′ of the hAAT sequence element that comprises a nucleotide sequence of SEQ ID NO: 29 or a variant of SEQ ID NO: 29 that differs by 1, 2, or 3 nucleotides; and/or
d. the TRE comprises a hAAT sequence element that comprises a nucleotide sequence of SEQ ID NO: 30 or a variant of SEQ ID NO: 30 that differs by 1, 2, or 3 nucleotides and the HNF6 binding site is 3′ of the hAAT sequence element that comprises a nucleotide sequence of SEQ ID NO: 30 or a variant of SEQ ID NO: 30 that differs by 1, 2, or 3 nucleotides; and/or
e. the TRE comprises a hAAT sequence element that comprises a nucleotide sequence of SEQ ID NO: 31 or a variant of SEQ ID NO: 31 that differs by 1, 2, or 3 nucleotides and the HNF6 binding site is 3′ of the hAAT sequence element that comprises a nucleotide sequence of SEQ ID NO: 31 or a variant of SEQ ID NO: 31 that differs by 1, 2, or 3 nucleotides; and/or
f. the TRE comprises a hAAT sequence element that comprises a nucleotide sequence of SEQ ID NO: 29 or a variant of SEQ ID NO: 29 that differs by 1, 2, or 3 nucleotides and the HNF6 binding site is 3′ of the hAAT sequence element that comprises a nucleotide sequence of SEQ ID NO: 29 or a variant of SEQ ID NO: 29 that differs by 1, 2, or 3 nucleotides; and/or
g. the TRE comprises an ApoE-HCR1 sequence element that comprises a nucleotide sequence of SEQ ID NO: 28 or a variant of SEQ ID NO: 28 that differs by 1, 2, or 3 nucleotides and the HNF6 binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
h. the TRE comprises an ApoE-HCR1 sequence element that comprises a nucleotide sequence of SEQ ID NO: 27 or a variant of SEQ ID NO: 27 that differs by 1, 2, or 3 nucleotides and the HNF6 binding site is 3′ of the ApoE-HCR1 sequence element that comprises a nucleotide sequence of SEQ ID NO: 27 or a variant of SEQ ID NO: 27 that differs by 1, 2, or 3 nucleotides; and/or
i. the TRE comprises an ApoE-HCR1 sequence element that comprises a nucleotide sequence of SEQ ID NO: 28 or a variant of SEQ ID NO: 28 that differs by 1, 2, or 3 nucleotides and the HNF6 binding site is 5′ of the ApoE-HCR1 sequence element that comprises a nucleotide sequence of SEQ ID NO: 28 or a variant of SEQ ID NO: 28 that differs by 1, 2, or 3 nucleotides; and/or
j. the TRE comprises an ApoE-HCR1 sequence element that comprises a nucleotide sequence of SEQ ID NO: 27 or a variant of SEQ ID NO: 27 that differs by 1, 2, or 3 nucleotides and the HNF6 binding site is 5′ of the ApoE-HCR1 sequence element that comprises a nucleotide sequence of SEQ ID NO: 27 or a variant of SEQ ID NO: 27 that differs by 1, 2, or 3 nucleotides; and/or
k. the TRE comprises an AMBP sequence element that comprises a nucleotide sequence of SEQ ID NO: 33 or a variant of SEQ ID NO: 33 that differs by 1, 2, or 3 nucleotides and the HNF6 binding site is 3′ of the AMBP sequence element that comprises a nucleotide sequence of SEQ ID NO: 33 or a variant of SEQ ID NO: 33 that differs by 1, 2, or 3 nucleotides; and/or
l. the TRE comprises an AMBP sequence element that comprises a nucleotide sequence of SEQ ID NO: 32 or a variant of SEQ ID NO: 32 that differs by 1, 2, or 3 nucleotides and the HNF6 binding site is 3′ of the AMBP sequence element that comprises a nucleotide sequence of SEQ ID NO: 32 or a variant of SEQ ID NO: 32 that differs by 1, 2, or 3 nucleotides; and/or
m. the TRE comprises an AMBP sequence element that comprises a nucleotide sequence of SEQ ID NO: 33 or a variant of SEQ ID NO: 33 that differs by 1, 2, or 3 nucleotides and the HNF6 binding site is 5′ of the AMBP sequence element that comprises a nucleotide sequence of SEQ ID NO: 33 or a variant of SEQ ID NO: 33 that differs by 1, 2, or 3 nucleotides; and/or
n. the TRE comprises an AMBP sequence element that comprises a nucleotide sequence of SEQ ID NO: 32 or a variant of SEQ ID NO: 32 that differs by 1, 2, or 3 nucleotides and the HNF6 binding site is 5′ of the AMBP sequence element that comprises a nucleotide sequence of SEQ ID NO: 32 or a variant of SEQ ID NO: 32 that differs by 1, 2, or 3 nucleotides.

The TRE may promote equivalent or greater expression than a corresponding TRE not comprising the HNF6 binding site.

In one aspect, a TRE comprising the HNF6 binding site promotes equivalent or greater expression than a corresponding TRE lacking an HNF6 binding site. The skilled person can determine whether a TRE comprising an HNF6 binding site (a test TRE) promotes equivalent or greater expression than a second TRE not comprising an HNF6 binding site (a reference TRE) by determining the level of expression promoted by the test TRE comprising an HNF6 binding site and the reference TRE using the techniques described under the heading “expression assay”. Optionally, the user determines whether a TRE comprising an HNF6 binding site (the test HNF6 binding site) promotes equivalent or greater expression than a reference TRE not comprising an HNF6 binding site by creating a test TRE that comprises the test HNF6 binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF6 binding site.

A TRE comprising a HNF6 binding site and a TATA box may promote equivalent or greater expression than a corresponding TRE not comprising the HNF6 binding site and the TATA box.

In an aspect, the TRE further comprises an HNF4a binding site. Optionally, the HNF4a binding site has a nucleotide sequence of SEQ ID NO: 88-91 as set out above.

In an aspect, the TRE comprises an HNF4a binding site, and:
a. the TRE comprises a hAAT sequence element that comprises a nucleotide sequence of SEQ ID NO: 30 or a variant of SEQ ID NO: 30 that differs by 1, 2, or 3 nucleotides and the HNF4a binding site is 5′ of the hAAT sequence element that comprises a nucleotide sequence of SEQ ID NO: 30 or a variant of SEQ ID NO: 30 that differs by 1, 2, or 3 nucleotides; and/or
b. the TRE comprises a hAAT sequence element that comprises a nucleotide sequence of SEQ ID NO: 31 or a variant of SEQ ID NO: 31 that differs by 1, 2, or 3 nucleotides and the HNF4a binding site is 5′ of the hAAT sequence element that comprises a nucleotide sequence of SEQ ID NO: 31 or a variant of SEQ ID NO: 31 that differs by 1, 2, or 3 nucleotides; and/or
c. the TRE comprises a hAAT sequence element that comprises a nucleotide sequence of SEQ ID NO: 29 or a variant of SEQ ID NO: 29 that differs by 1, 2, or 3 nucleotides and the HNF4a binding site is 5′ of the hAAT sequence element that comprises a nucleotide sequence of SEQ ID NO: 29 or a variant of SEQ ID NO: 29 that differs by 1, 2, or 3 nucleotides; and/or
d. the TRE comprises an ApoE-HCR1 sequence element that comprises a nucleotide sequence of SEQ ID NO: 28 or a variant of SEQ ID NO: 28 that differs by 1, 2, or 3 nucleotides and the HNF4a binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
e. the TRE comprises an ApoE-HCR1 sequence element that comprises a nucleotide sequence of SEQ ID NO: 28 or a variant of SEQ ID NO: 28 that differs by 1, 2, or 3 nucleotides and the HNF4a binding site is 5′ of the ApoE-HCR1 sequence element that comprises a nucleotide sequence of SEQ ID NO: 28 or a variant of SEQ ID NO: 28 that differs by 1, 2, or 3 nucleotides; and/or
f. the TRE comprises an ApoE-HCR1 sequence element that comprises a nucleotide sequence of SEQ ID NO: 27 or a variant of SEQ ID NO: 27 that differs by 1, 2, or 3 nucleotides and the HNF4a binding site is 5′ of the ApoE-HCR1 sequence element that comprises a nucleotide sequence of SEQ ID NO: 27 or a variant of SEQ ID NO: 27 that differs by 1, 2, or 3 nucleotides; and/or
g. the TRE comprises an AMBP sequence element that comprises a nucleotide sequence of SEQ ID NO: 33 or a variant of SEQ ID NO: 33 that differs by 1, 2, or 3 nucleotides and the HNF4a binding site is 3′ of the AMBP sequence element that comprises a nucleotide sequence of SEQ ID NO: 33 or a variant of SEQ ID NO: 33 that differs by 1, 2, or 3 nucleotides; and/or
h. the TRE comprises an AMBP sequence element that comprises a nucleotide sequence of SEQ ID NO: 32 or a variant of SEQ ID NO: 32 that differs by 1, 2, or 3 nucleotides and the HNF4a binding site is 3′ of the AMBP sequence element that comprises a nucleotide sequence of SEQ ID NO: 32 or a variant of SEQ ID NO: 32 that differs by 1, 2, or 3 nucleotides; and/or
i. the TRE comprises an AMBP sequence element that comprises a nucleotide sequence of SEQ ID NO: 33 or a variant of SEQ ID NO: 33 that differs by 1, 2, or 3 nucleotides and the HNF4a binding site is 5′ of the AMBP sequence element that comprises a nucleotide sequence of SEQ ID NO: 33 or a variant of SEQ ID NO: 33 that differs by 1, 2, or 3 nucleotides; and/or
j. the TRE comprises an AMBP sequence element that comprises a nucleotide sequence of SEQ ID NO: 32 or a variant of SEQ ID NO: 32 that differs by 1, 2, or 3 nucleotides and the HNF4a binding site is 5′ of the AMBP sequence element that comprises a nucleotide sequence of SEQ ID NO: 32 or a variant of SEQ ID NO: 32 that differs by 1, 2, or 3 nucleotides.

The TRE may provide equivalent or greater expression than a corresponding TRE not comprising the HNF4a binding site.

In one aspect, a TRE comprising the HNF4a binding site promotes equivalent or greater expression than a corresponding TRE lacking an HNF4a binding site. The skilled person can determine whether a TRE comprising an HNF4a binding site (a test TRE) promotes equivalent or greater expression than a second TRE not comprising an HNF4a binding site (a reference TRE) by determining the level of expression promoted by the test TRE comprising an HNF4a binding site and the reference TRE using the techniques described under the heading “expression assay”. Optionally, the user determines whether a TRE comprising an HNF4a binding site (the test HNF4a binding site) promotes equivalent or greater expression than a reference TRE not comprising an HNF4a binding site by creating a test TRE that comprises the test HNF4a binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF4a binding site.

A TRE comprising a HNF6 binding site and an HNF4a binding site may promote equivalent or greater expression than a corresponding TRE not comprising the HNF6 binding site and the HNF4a binding site.

In an aspect, the TRE further comprises an HNF1 binding site. Optionally, the HNF1 binding site has a nucleotide sequence of SEQ ID NO: 70 or 71 as set out above.

In an aspect, the TRE comprises an HNF1 binding site, and:
a. the TRE comprises a hAAT sequence element that comprises a nucleotide sequence of SEQ ID NO: 30 or a variant of SEQ ID NO: 30 that differs by 1, 2, or 3 nucleotides and the HNF1 binding site is 5′ of the hAAT sequence element that comprises a nucleotide sequence of SEQ ID NO: 30 or a variant of SEQ ID NO: 30 that differs by 1, 2, or 3 nucleotides; and/or
b. the TRE comprises a hAAT sequence element that comprises a nucleotide sequence of SEQ ID NO: 31 or a variant of SEQ ID NO: 31 that differs by 1, 2, or 3 nucleotides and the HNF1 binding site is 5′ of the hAAT sequence element that comprises a nucleotide sequence of SEQ ID NO: 31 or a variant of SEQ ID NO: 31 that differs by 1, 2, or 3 nucleotides; and/or
c. the TRE comprises a hAAT sequence element that comprises a nucleotide sequence of SEQ ID NO: 29 or a variant of SEQ ID NO: 29 that differs by 1, 2, or 3 nucleotides and the HNF1 binding site is 5′ of the hAAT sequence element that comprises a nucleotide sequence of SEQ ID NO: 29 or a variant of SEQ ID NO: 29 that differs by 1, 2, or 3 nucleotides; and/or
d. the TRE comprises an ApoE-HCR1 sequence element that comprises a nucleotide sequence of SEQ ID NO: 28 or a variant of SEQ ID NO: 28 that differs by 1, 2, or 3 nucleotides and the HNF1 binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
e. the TRE comprises an ApoE-HCR1 sequence element that comprises a nucleotide sequence of SEQ ID NO: 27 or a variant of SEQ ID NO: 27 that differs by 1, 2, or 3 nucleotides and the HNF1 binding site is 3′ of the ApoE-HCR1 sequence element that comprises a nucleotide sequence of SEQ ID NO: 27 or a variant of SEQ ID NO: 27 that differs by 1, 2, or 3 nucleotides; and/or
f. the TRE comprises an AMBP sequence element that comprises a nucleotide sequence of SEQ ID NO: 33 or a variant of SEQ ID NO: 33 that differs by 1, 2, or 3 nucleotides and the HNF1 binding site is 3′ of the AMBP sequence element that comprises a nucleotide sequence of SEQ ID NO: 33 or a variant of SEQ ID NO: 33 that differs by 1, 2, or 3 nucleotides; and/or g the TRE comprises an AMBP sequence element that comprises a nucleotide sequence of SEQ ID NO: 32 or a variant of SEQ ID NO: 32 that differs by 1, 2, or 3 nucleotides and the HNF1 binding site is 3′ of the AMBP sequence element that comprises a nucleotide sequence of SEQ ID NO: 32 or a variant of SEQ ID NO: 32 that differs by 1, 2, or 3 nucleotides.

The TRE may provide equivalent or greater expression than a corresponding TRE not comprising the HNF1 binding site.

In one aspect, a TRE comprising the HNF1 binding site promotes equivalent or greater expression than a corresponding TRE lacking an HNF1 binding site. The skilled person can determine whether a TRE comprising an HNF1 binding site (a test TRE) promotes equivalent or greater expression than a second TRE not comprising an HNF1 binding site (a reference TRE) by determining the level of expression promoted by the test TRE comprising an HNF1 binding site and the second TRE using the techniques described under the heading “expression assay”. Optionally, the user determines whether a TRE comprising an HNF1 binding site (the test HNF1 binding site) promotes equivalent or greater expression than a reference TRE not comprising an HNF1 binding site by creating a test TRE that comprises the test HNF1 binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF1 binding site.

A TRE comprising an HNF6 binding site, an HNF4a binding site, and a TATA box may promote equivalent or greater expression than a corresponding TRE not comprising the HNF6 binding site, the HNF4a binding site, and the TATA box.

In an aspect, the TRE further comprises an HNF3 binding site. Optionally, the HNF3 binding site has a nucleotide sequence of SEQ ID NO: 79 to 81 as set out above.

In an aspect, the TRE comprises an HNF3 binding site, and:
a. the TRE comprises a hAAT sequence element that comprises a nucleotide sequence of SEQ ID NO: 30 or a variant of SEQ ID NO: 30 that differs by 1, 2, or 3 nucleotides and the HNF3 binding site is 5′ of the hAAT sequence element that comprises a nucleotide sequence of SEQ ID NO: 30 or a variant of SEQ ID NO: 30 that differs by 1, 2, or 3 nucleotides; and/or
b. the TRE comprises a hAAT sequence element that comprises a nucleotide sequence of SEQ ID NO: 31 or a variant of SEQ ID NO: 31 that differs by 1, 2, or 3 nucleotides and the HNF3 binding site is 5′ of the hAAT sequence element that comprises a nucleotide sequence of SEQ ID NO: 31 or a variant of SEQ ID NO: 31 that differs by 1, 2, or 3 nucleotides; and/or
c. the TRE comprises a hAAT sequence element that comprises a nucleotide sequence of SEQ ID NO: 29 or a variant of SEQ ID NO: 29 that differs by 1, 2, or 3 nucleotides and the HNF3 binding site is 5′ of the hAAT sequence element that comprises a nucleotide sequence of SEQ ID NO: 29 or a variant of SEQ ID NO: 29 that differs by 1, 2, or 3 nucleotides; and/or
d. the TRE comprises an ApoE-HCR1 sequence element that comprises a nucleotide sequence of SEQ ID NO: 28 or a variant of SEQ ID NO: 28 that differs by 1, 2, or 3 nucleotides and the HNF3 binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
e. the TRE comprises an ApoE-HCR1 sequence element that comprises a nucleotide sequence of SEQ ID NO: 27 or a variant of SEQ ID NO: 27 that differs by 1, 2, or 3 nucleotides and the HNF3 binding site is 3′ of the ApoE-HCR1 sequence element that comprises a nucleotide sequence of SEQ ID NO: 27 or a variant of SEQ ID NO: 27 that differs by 1, 2, or 3 nucleotides; and/or
f. the TRE comprises an AMBP sequence element that comprises a nucleotide sequence of SEQ ID NO: 33 or a variant of SEQ ID NO: 33 that differs by 1, 2, or 3 nucleotides and the HNF3 binding site is 3′ of the AMBP sequence element that comprises a nucleotide sequence of SEQ ID NO: 33 or a variant of SEQ ID NO: 33 that differs by 1, 2, or 3 nucleotides; and/or g the TRE comprises an AMBP sequence element that comprises a nucleotide sequence of SEQ ID NO: 32 or a variant of SEQ ID NO: 32 that differs by 1, 2, or 3 nucleotides and the HNF3 binding site is 3′ of the AMBP sequence element that comprises a nucleotide sequence of SEQ ID NO: 32 or a variant of SEQ ID NO: 32 that differs by 1, 2, or 3 nucleotides.

The TRE may provide equivalent or greater expression than a corresponding TRE not comprising the HNF3 binding site.

In one aspect, a TRE comprising the HNF3 binding site promotes equivalent or greater expression than a corresponding TRE lacking an HNF3 binding site. The skilled person can determine whether a TRE comprising an HNF3 binding site (a test TRE) promotes equivalent or greater expression than a second TRE not comprising an HNF3 binding site (a reference TRE) by determining the level of expression promoted by the test TRE and the reference TRE using the techniques described under the heading “expression assay”. Optionally, the user determines whether a TRE comprising an HNF3 binding site (the test HNF3 binding site) promotes equivalent or greater expression than a reference TRE not comprising an HNF3 binding site by creating a test TRE that comprises the test HNF3 binding site 5′ of HCR-hAAT and comparing the level of expression of the test TRE with that of HCR-hAAT (the reference TRE). If the level of expression of the test TRE is equivalent or higher than the level of expression of HCR-hAAT, then the TRE promotes equivalent or greater expression than a corresponding TRE not comprising an HNF3 binding site.

A TRE comprising an HNF3 binding site and a TATA box and the TRE may promote equivalent or greater expression than a corresponding TRE not comprising the HNF3 binding site and the TATA box.

### Exemplary TRES

In one embodiment, the TRE comprises:
(i) an AMBP sequence element:
(ii) a hAAT sequence element:
(iii) an ApoE-HCR1 sequence element:
(iv) a Transcription Start Site (TSS); and
(v) a TATA box,
   wherein:
(i) the AMBP sequence element comprises a nucleotide sequence of SEQ ID NO: 32:
(ii) the hAAT sequence element comprises a nucleotide sequence of SEQ ID NO: 29:
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element:
(vi) the TATA box is at position −37 from the TSS; and
(vii) the TRE is between 875 and 925 nucleotides in length.

In one embodiment, the TRE comprises:
(i) an AMBP sequence element:
(ii) a hAAT sequence element;
(iii) an ApoE-HCR1 sequence element;
(iv) an HNF6 binding site;
(v) an HNF4a binding site:
(vi) a TATA box; and
(vii) a second HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27:
(iv) the AMBP sequence element is 5′ of the hAAT sequence element:
(v) the ApoE-HCR1 sequence element is 5′ of the hAAT sequence element and 3′ of the AMBP sequence element;
(vi) the HNF6 binding site is 3′ of the AMBP sequence element and 5′ of the ApoE-HCR1 sequence element:
(vii) the HNF4a binding site is 3′ of the ApoE-HCR-1 sequence element and 5′ of the hAAT sequence element:
(viii) the TATA box is immediately 5′ of a nucleotide sequence corresponding to the nucleotide sequence of SEQ ID NO: 30;
(ix) the second HNF6 binding site is immediately 3′ of a nucleotide sequence corresponding to the nucleotide sequence of SEQ ID NO: 30; and
(x) the TRE is between 900 and 950 nucleotides in length.

In one embodiment, the TRE comprises:
(i) an AMBP sequence element:
(ii) a hAAT sequence element;
(iii) an ApoE-HCR1 sequence element:
(iv) an HNF6 binding site;
(v) an HNF1 binding site:
(vi) an HNF4a binding site;
(vii) a TATA box; and
(viii) a second HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element:
(vi) the HNF6 binding site is 3′ of the AMBP sequence element and 5′ of the ApoE-HCR1 sequence element;
(vii) the HNF1 binding site is immediately 3′ of the HNF4a binding site;
(viii) HNF4a binding site is 3′ of the ApoE-HCR-1 sequence element and 5′ of the hAAT sequence element;
(ix) the TATA box is immediately 5′ of a nucleotide sequence corresponding to the nucleotide sequence of SEQ ID NO: 30;
(x) the second HNF6 binding site is immediately 3′ of a nucleotide sequence corresponding to the nucleotide sequence of SEQ ID NO: 30; and
(xi) the TRE is between 900 and 950 nucleotides in length.

In one embodiment, the TRE comprises:
(i) an AMBP sequence element;
(ii) a hAAT sequence element;
(iii) an ApoE-HCR1 sequence element;
(iv) an HNF3 binding site; and
(v) a TATA box,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF3 binding site is 3′ of the ApoE-HCR1 sequence element and 5′ of the hAAT sequence element;
(vii) the TATA box is immediately 5′ of a nucleotide sequence corresponding to the nucleotide sequence of SEQ ID NO: 30; and
(viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) a TATA box,
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27:
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is 3′ of the AMBP sequence element and 5′ of the ApoE-HCR1 sequence element;
(vii) the TATA box is immediately 5′ of a nucleotide sequence corresponding to the nucleotide sequence of SEQ ID NO: 30; and
(viii) the TRE is between 875 and 925 nucleotides in length.

In one embodiment, the TRE comprises:
(i) an AMBP sequence element:
(ii) a hAAT sequence element;
(iii) an ApoE-HCR1 sequence element; and
(iv) an HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is 3′ of the AMBP sequence element and 5′ of the ApoE-HCR1 sequence element; and
(vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF4 binding site is 3′ of the AMBP sequence element and 5′ of the ApoE-HCR1 sequence element; and
(vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is 3′ of the ApoE-HCR1 sequence element and 5′ of the hAAT sequence element; and
(vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF4 binding site is 3′ of the ApoE-HCR1 sequence element and 5′ of the hAAT sequence element; and
(vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site,
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF4 binding site is immediately 3′ of a nucleotide sequence corresponding to a nucleotide sequence of SEQ ID NO: 28; and
(vii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) a TATA box,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is immediately 3′ of a nucleotide sequence corresponding to the nucleotide sequence of SEQ ID NO: 30;
(vii) the TATA box is immediately 5′ of the HNF6 binding site; and
(viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF4 binding site; and
(v) an HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF4 binding site is 3′ of the ApoE-HCR1 sequence element and 5′ of the hAAT sequence element;
(vii) the HNF6 binding site is 3′ of the AMBP sequence element and 5′ of the ApoE-HCR1 sequence element; and
(viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) an HNF4 binding site,
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is 3′ of the ApoE-HCR1 sequence element and 5′ of the hAAT sequence element;
(vii) the HNF4 binding site is 3′ of the AMBP sequence element and 5′ of the ApoE-HCR1 sequence element; and
(viii) the TRE is between 875 and 925 nucleotides in length.

In one embodiment, the TRE comprises:
(i) an AMBP sequence element;
(ii) a hAAT sequence element;
(iii) an ApoE-HCR1 sequence element;
(iv) an HNF6 binding site; and
(v) a TATA box,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is immediately 3′ of a nucleotide sequence corresponding to the nucleotide sequence of SEQ ID NO: 30;
(vii) the TATA box is immediately 5′ of a nucleotide sequence corresponding to the nucleotide sequence of SEQ ID NO: 30; and
(viii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is 5′ of the AMBP sequence element; and
(vii) the TRE is between 875 and 925 nucleotides in length.

In one embodiment, the TRE comprises:
(i) an AMBP sequence element;
(ii) a hAAT sequence element;
(iii) an ApoE-HCR1 sequence element; and
(iv) an HNF4 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF4 binding site is 5′ of the AMBP sequence element; and
(vii) the TRE is between 875 and 925 nucleotides in length.

In one embodiment, the TRE comprises:
(i) an AMBP sequence element:
(ii) a hAAT sequence element:
(iii) an ApoE-HCR1 sequence element; and
(iv) an HNF6 binding site,
   wherein:
(i) the AMBP sequence element comprises a nucleotide sequence of SEQ ID NO: 32;
(ii) the hAAT sequence element comprises a nucleotide sequence of SEQ ID NO: 29;
(iii) the ApoE-HCR1 sequence element comprises a nucleotide sequence of SEQ ID NO: 27;
(iv) the AMBP sequence element is 5′ of the hAAT sequence element;
(v) the ApoE-HCR1 sequence element is 5′ of the hAAT sequence element and 3′ of the AMBP sequence element;
(vi) the HNF6 binding site is immediately 3′ of a nucleotide sequence corresponding to the nucleotide sequence of SEQ ID NO: 30; and
(vii) the TRE is between 875 and 925 nucleotides in length.

### Expression Cassette

In one aspect, the invention provides an expression cassette comprising a TRE of the invention operably linked to a transgene. The transgene may be at least 100, at least 500, at least 1000, at least 1500, at least 2000, at least 2500, at least 3000, at least 3500, at least 4000, at least 4500, or at least 5000 nucleotides in length. The transgene may be less than 5000, less than 4500, less than 4000, less than 3500, less than 3000, less than 2500, less than 2000, less than 1500, less than 1000, less than 500, or less than 100 nucleotides in length. The transgene may be between 100 and 5000, between 500 and 5000, between 1000 and 5000, between 1500 and 5000, between 2000 and 5000, between 3000 and 5000, between 3500 and 5000, between 4000 and 5000, or between 4500 and 5000 nucleotides in length.

The skilled person understands that different viruses have viral genomes of different sizes. Furthermore, the skilled person understands that the size of the expression cassette may affect the stability of the virus and that concomitantly transduction efficiency of a viral vector may decrease. In the case that the size of the expression cassette would result in a viral genome that is too small, stuffer sequences can be used to increase the size of the expression cassette. However, stuffer sequences are known to have unpredictable effects on the stability and transduction of viral vectors. It is therefore desirable to be able to control the size of the expression cassette by using a panel of differently sized TREs.

Accordingly, in one aspect, the present invention provides an expression cassette comprising a TRE operably linked to a transgene, wherein the size of the TRE used in the expression cassette is selected based on the size of the transgene used in the expression cassette. In an aspect, the present invention provides an expression cassette comprising a TRE operably linked to a transgene, wherein the combined size of the TRE and transgene does not exceed a limit placed upon it by the size of a viral vector genome (for example the combined size of the TRE, the transgene and any other necessary sequences such as ITR sequences do not exceed the wild type AAV genome size). In an aspect, the present invention provides an expression cassette comprising a TRE operably linked to a transgene, wherein the combined size of the TRE and transgene reduces the need for stuffer sequences.

In one embodiment, the transgene does not encode a FVIII polypeptide. For example, in some embodiments, the transgene does not encode a wild-type FVIII polypeptide, such as the FVIII polypeptide of SEQ ID NO: 141. In one embodiment, the transgene of the invention does not encode a contiguous polypeptide with greater than 90%, greater than 95%, or greater than 98% sequence identity to SEQ ID NO: 141. In one embodiment, the transgene does not encode a FVIII polypeptide of SEQ ID NO: 141.

### A Vector Comprising the Expression Cassette

In one aspect of the invention, there is provided a vector comprising a recombinant vector genome comprising an expression cassette of the invention. In some aspects, the vector is a viral particle. In other aspects, the vector is a plasmid.

### A Viral Particle Comprising the TRE

The invention further provides a viral particle comprising a recombinant genome comprising a TRE or expression cassette of the invention. For the purposes of the present invention, the term “viral particle” refers to all or part of a virion. For example, the viral particle comprises a recombinant genome and may further comprise a capsid. The viral particle may be a gene therapy vector. Herein, the terms “viral particle” and “vector” are used interchangeably. For the purpose of the present invention, a “gene therapy” vector is a viral particle that can be used in gene therapy, i.e. a viral particle that comprises all the required functional elements to express a transgene, such as a CFI nucleotide sequence, in a host cell after administration.

Suitable viral particles include a parvovirus, a retrovirus, a lentivirus, or a herpes simplex virus. The parvovirus may be an adeno-associated virus (AAV). The viral particle is preferably a recombinant adeno-associated viral (AAV) vector or a lentiviral vector. More preferably, the viral particle is an AAV viral particle. The terms AAV and rAAV are used interchangeably herein, unless the context indicates otherwise.

The genomic organization of all known AAV serotypes is very similar. The genome of AAV (i.e. the vector genome) is a linear, single-stranded DNA molecule that is less than about 5,000 nucleotides in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the vector genome. A splicing event in the Rep ORF results in the expression of four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

The viral particle of the invention may comprise a vector genome that comprise ITRs. It is possible for an AAV viral particle/vector of the invention to function with only one ITR. Thus, the vector genome comprises at least one ITR, but, more typically, two ITRs (generally with one either end of the viral genome, i.e. one at the 5′ end and one at the 3′ end). There may be intervening sequences between the expression cassette and one or more of the ITRs. Optionally, there are fewer than 10, fewer than 5, fewer than 3, or fewer than 1 nucleotide(s) between the expression cassette and the or both ITR(s). The expression cassette of the invention may be incorporated into a viral particle located between two regular ITRs or located on either side of an ITR engineered with two D regions.

AAV sequences that may be used in the present invention for the production of AAV vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790: GenBank Accession number J01901: GenBank Accession number AF043303: GenBank Accession number AF085716: Chiorini et al, 1997; Srivastava et al, 1983; Chiorini et al, 1999: Rutledge et al, 1998; and Wu et al, 2000. AAV serotype 1, 2, 3, 3B, 4, 5, 6, 7, 8, 9, 10, 11 or 12 may be used in the present invention. The sequences from the AAV serotypes may be mutated or engineered when being used in the production of gene therapy vectors.

Optionally, an AAV vector/viral particle comprises ITR sequences which are derived from AAV1, AAV2, AAV4 and/or AAV6. Preferably the ITR sequences are AAV2 ITR sequences. Herein, the term AAVx/y refers to a viral particle that comprises some components from AAVx (wherein x is a AAV serotype number) and some components from AAVy (wherein y is the number of the same or different serotype). For example, an AAV2/8 vector may comprise a portion of a viral genome, including the ITRs, from an AAV2 strain, and a capsid derived from an AAV8 strain.

In an embodiment, the viral particle is an AAV viral particle comprising a capsid. AAV capsids are generally formed from three proteins, VP1, VP2 and VP3. The amino acid sequence of VP1 comprises the sequence of VP2. The portion of VP1 which does not form part of VP2 is referred to as VPlunique or VPIU. The amino acid sequence of VP2 comprises the sequence of VP3. The portion of VP2 which does not form part of VP3 is referred to as VP2unique or VP2U. Preferably the capsid is an AAV8 capsid, AAV5 capsid or a Mut C capsid, such as that disclosed in WO2016/181123.

A viral particle of the invention may be a “hybrid” particle in which the viral ITRs and viral capsid are from different parvoviruses, such as different AAV serotypes. Preferably, the viral ITRs and capsid are from different serotypes of AAV, in which case such viral particles are known as transcapsidated or pseudotyped. Likewise, the parvovirus may have a “chimeric” capsid (e.g., containing sequences from different parvoviruses, preferably different AAV serotypes) or a “targeted” capsid (e.g., a directed tropism).

In some embodiments, the vector genome comprises intact ITRs, comprising functional terminal resolution sites (TRS). Such a vector genome may contain one or two resolvable ITRs, i.e. ITRs containing a functional TRS at which site-specific nicking can take place to create a free 3′ hydroxyl group which can serve as a substrate for DNA polymerase to unwind and copy the ITR. Preferably, the vector genome is single-stranded (i.e., it is packaged into the viral particle in a single-stranded form). Optionally, the vector genome is not packaged in self-complementary configuration, i.e. the genome does not comprise a single covalently-linked polynucleotide strand with substantial self-complementary portions that anneal in the viral particle. Alternatively, the vector genome may be packaged in “monomeric duplex” form. “Monomeric duplexes” are described in WO 2011/122950. The genome may be packaged as two substantially complementary but non-covalently linked polynucleotides which anneal in the viral particle.

The viral particle/vector may further comprise a poly A sequence. The poly A sequence may be positioned downstream of the nucleotide sequence encoding a protein. The poly A sequence may be a bovine growth hormone poly A sequence (bGHpA). The poly A sequence may be between 250 and 270 nucleotides in length.

### Compositions, Methods and Uses

In a further aspect of the invention, there is provided a composition comprising a vector/viral particle comprising the TRE or expression cassette of the invention and a pharmaceutically acceptable excipient.

The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

The invention further provides an AAV vector/viral particle of the invention for use in a method of treatment/method of treating a disease. Optionally, the method of treatment comprises administering an effective amount of the AAV vector/viral particle of the invention to a patient.

The invention further provides a method of treatment comprising administering an effective amount of the AAV vector/viral particle of the invention to a patient.

The invention further provides use of the AAV vector/viral particle or composition of the invention in the manufacture of a medicament for use in a method of treatment/method of treating a disease. For the avoidance of doubt, the terms “method of treating” and “method of treating a disease are used interchangeably herein. Optionally, the method of treatment/method of treating a disease comprises administering an effective amount of the composition or AAV vector/viral particle of the invention to a patient. Optionally, the method of treatment/method of treating a disease is a gene therapy. A “gene therapy” involves administering a vector/viral particle of the invention that is capable of expressing a transgene (such as a polynucleotide of the invention) in the host to which it is administered.

Optionally, the method of treatment is a method of treating a genetic disorder, optionally by gene therapy. Optionally, the genetic disorder is a complement-mediated disorder. Optionally, the complement-mediated disorder is selected from C3 glomerulopathy, IgA nephropathy, lupus nephritis, systemic lupus erythematosus, membranous nephropathy, membranoproliferative glomerulonephritis, paroxysmal nocturnal hemoglobinuria, atypical hemolytic uremic syndrome, autoimmune haemolytic anemia, ANCA-associated vasculitis, Gaucher disease, peritonitis, age-related macular degeneration, diabetic retinopathy, dense deposit disease, age-related inflammatory or autoinflammatory diseases, autoimmune arthritis such as rheumatoid arthritis, atherosclerosis, chronic cardiovascular disease, Alzheimer's disease, systemic vasculitis, Guillain-Barre syndrome, and Henoch-Schonlein purpura.

Optionally, the complement-mediated disorder is a kidney glomerular or tubular disorder. Optionally, the disorder is selected from C3 glomerulopathy, IgA nephropathy, lupus nephritis and membranous nephropathy. Optionally, the disorder is lupus nephritis. Optionally, the disorder is systemic lupus erythematosus.

Optionally, the composition and/or AAV vector/viral particle is administered intravenously. Optionally, the composition and/or AAV vector/viral particle is for administration only once (i.e. a single dose) to a patient.

When a genetic disorder is “treated” in the above method, this means that one or more symptoms of the genetic disorder are ameliorated. It does not mean that the symptoms of the genetic disorder are completely remedied so that they are no longer present in the patient, although in some methods, this may be the case. The method of treatment may result in one or more of the symptoms of the genetic disorder being less severe than before treatment. Optionally, relative to the situation pre-administration, the method of treatment results in an increase in the amount/concentration of circulating transgene in the blood of the patient, and/or the overall level of transgene activity detectable within a given volume of blood of the patient, and/or the specific activity (activity per amount of transgene) of the transgene in the blood of the patient.

A “therapeutically effective amount” refers to an amount effective, at dosages and for periods of time necessary, to achieve the desired therapeutic result, such as raising the level of a transgene in a subject (so as to lead to functional transgene production at a level sufficient to ameliorate the symptoms of a genetic disorder).

Optionally, the AAV vector/viral particle is administered at a dose of less than 1×10¹¹, less than 1×10¹², less than 5×10¹², less than 2×10¹², less than 1.5×10¹², less than 3×10¹², less than 1×10¹³, less than 2×10¹³, or less than 3×10¹³ vector genomes per kg of weight of patient.

### EXAMPLE

### Example 1—Huh7 In Vitro Transfection Study of Candidate Promoters

Novel hAAT-based TREs were screened and ranked based on the amount (w/v) of protein produced after transient transfection of Huh7 cells. Each of the novel hAAT-based TREs was conjugated to a wild-type human CFI polypeptide (the wild-type human CFI polypeptide having the sequence of SEQ ID NO: 41).

### Methods

Expression cassettes were generated which comprise one of the novel hAAT-based TREs (each TRE having a sequence of one of SEQ ID NO: 3-26) or HCR-hAAT (SEQ ID NO: 2), HLP2 (SEQ ID NO: 4342 or FRE72 (SEQ ID NO: 1) for control purposes.

The transient transfection entailed mixing, for each expression cassette comprising one of the TREs, a plasmid carrying such expression cassette with a transfection reagent (FuGENERHD (Promega, cat no. E2311)) and delivering the mixture to cells so that the gene can be expressed and protein subsequently quantified by ELISA. In each case, a luciferase reporter gene was included in the transfection mixture as a way of evaluating transfection efficiency.

Huh7 cell culture and Transfection: Huh7 cells (JCRB cell bank, no. JCRB0403) were seeded in a 96 well plate (30,000 cells per well) in DMEM low glucose, 10% FBS+Glutamax (D10 media) and cultured at 37° ° C. and 5% CO₂ (day 1). The next day (approx. 24 hours after cell seeding: day 2), the plasmid DNA-transfection reagent mixture was prepared and transfected into Huh7 cells. Briefly, 0.225 μg of test plasmid DNA and 0.025 μg CMV-Luciferase control plasmid (FLJ-PL282) were mixed with FuGENE at a ratio of 4 μl FuGENE per μg of DNA (or 1 μl FuGENE per 0.25 μg of plasmid DNA). In addition, 0.25 μg of CMV-Luciferase control plasmid (FLJ-PL282) was mixed with FuGENE at a ratio of 4 μl FuGENE per μg of DNA to be used as a positive control for the luciferase assay. For 96-well transfection experiments, 10 μl of the plasmid DNA-FuGENE mix was added per well. The plasmid DNA-transfection reagent mixture was incubated on the cells overnight at 37° C. and 5% CO₂. The next morning (day 3), approximately 18 hours after transfection, the media was replaced with fresh D10 media and cells incubated overnight at 37° C. and 5% CO₂. The next morning (24h later: day 4), media was replaced by fresh DMEM low glucose+Glutamax+Insulin-Transferrin-Selenium supplement (DO/ITS media). Cells and media were harvested the following day (on day 5).

CFI expression in culture media was assessed using a CFI ELISA kit (e.g. Hycult cat#HK355-01) according to manufacturer's instructions. In brief, culture media samples (diluted with the sample diluent from the kit as necessary) and human CFI standards from the kit were incubated in microtiter wells coated with anti-human CFI antibodies (to capture the human CFI). A biotinylated tracer antibody was added (to bind to the captured human CFI) followed by a streptavidin-peroxidase conjugate (to bind to the biotinylated tracer antibody). The substrate tetramethylbenzidine (TMB) was then added, which the streptavidin-peroxidase conjugate reacts with. The enzyme reaction was stopped by adding oxalic acid. For each of the samples and standards, the absorbance at 450 nm was measured with a spectrophotometer. A standard curve was obtained by plotting the absorbance (linear) against the corresponding concentrations of the human CFI standards (log). The concentration of human CFI in the samples was then determined from the standard curve.

In parallel to the ELISA, Huh7 cells were washed with phosphate buffered saline (PBS) twice and cells treated with 100 μl of luciferase lysis buffer from the Luciferase assay kit (Promega cat #E1501/E4530). Cell lysates were stored at −80° ° C. On the day of luciferase assays, cell lysates were thawed and 20 μl of the sample was used to measure the luciferase expression by luminescence on a Molecular Devices SpectraMax i3x plate reader. The detailed protocol is published in the Promega Technical Bullitin #TB281. Where applicable, luciferase expression was used as internal control to normalise the CFI expression levels. Analyses were performed using the software Graphpad Prism v7.

### Results

The CFI expression levels in the culture media following transfection into Huh-7 cells were measured for HCR-hAAT, HLP2, FRE72, and twenty-four other hAAT-based TREs.

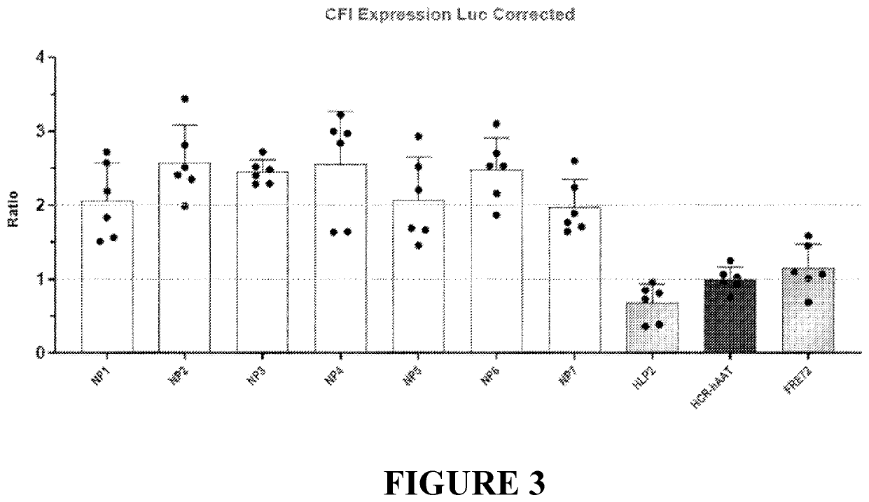

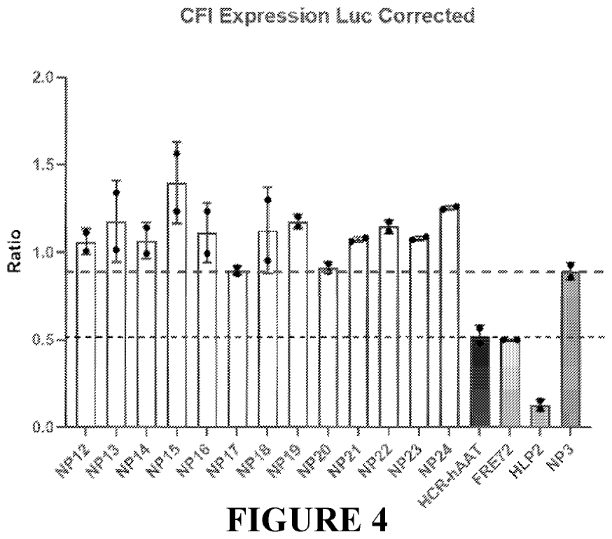

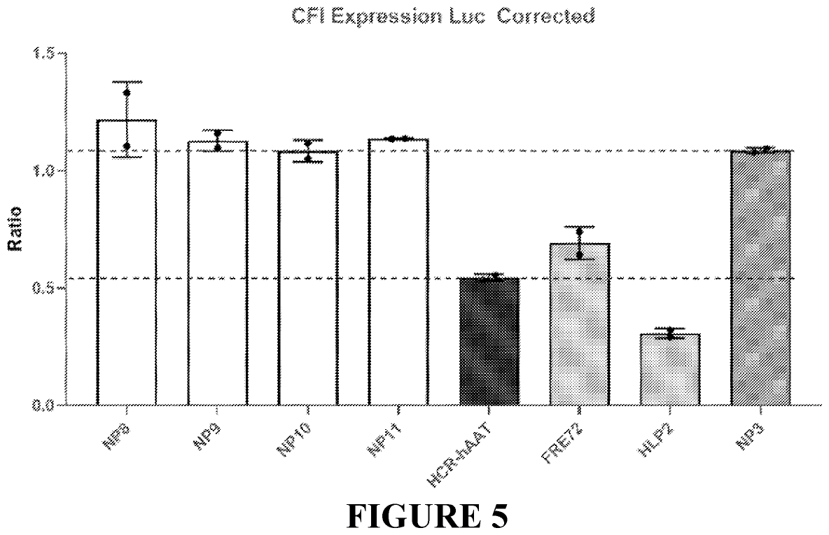

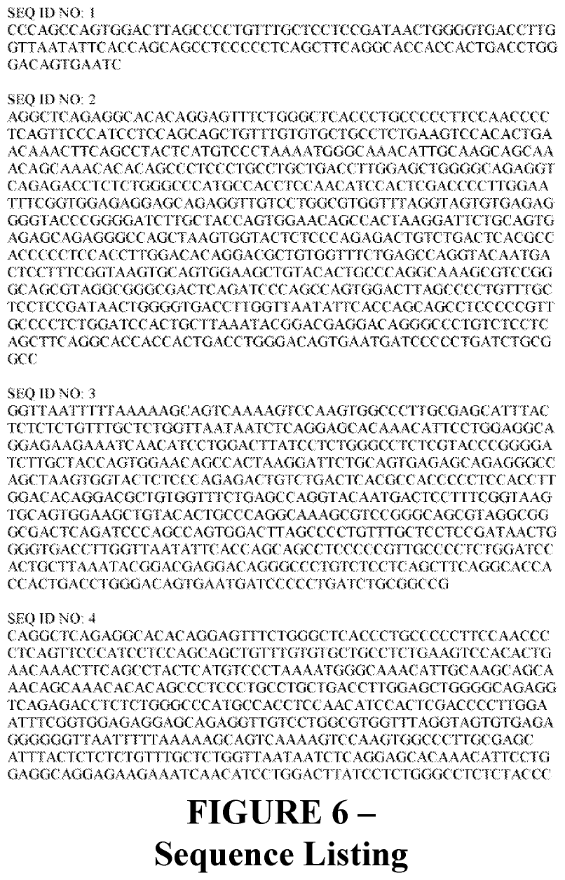

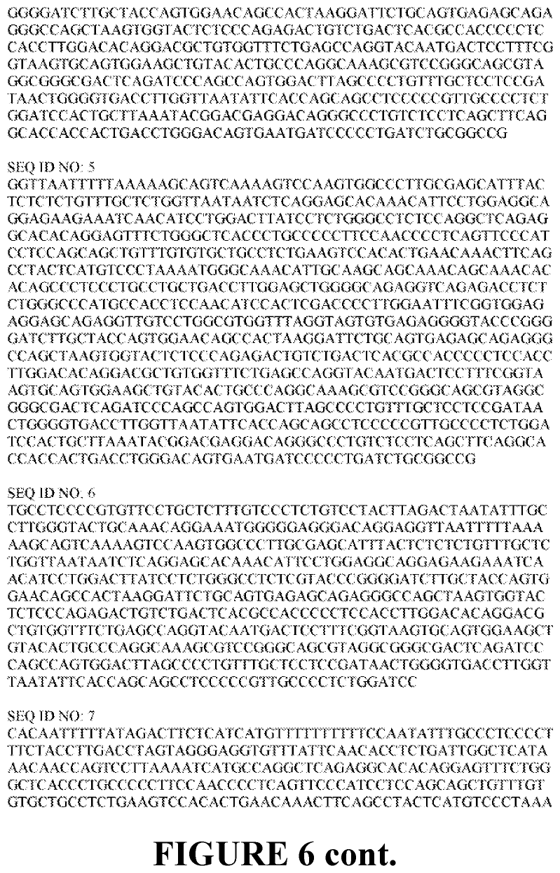

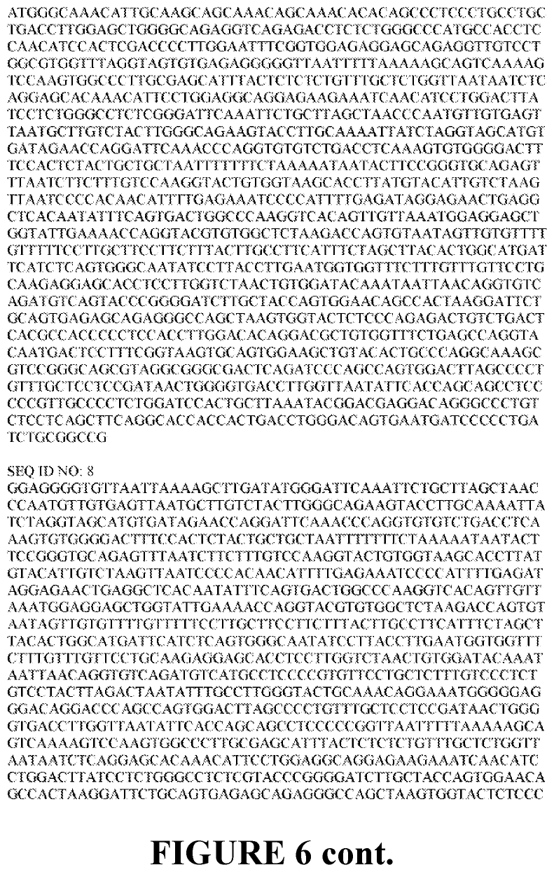

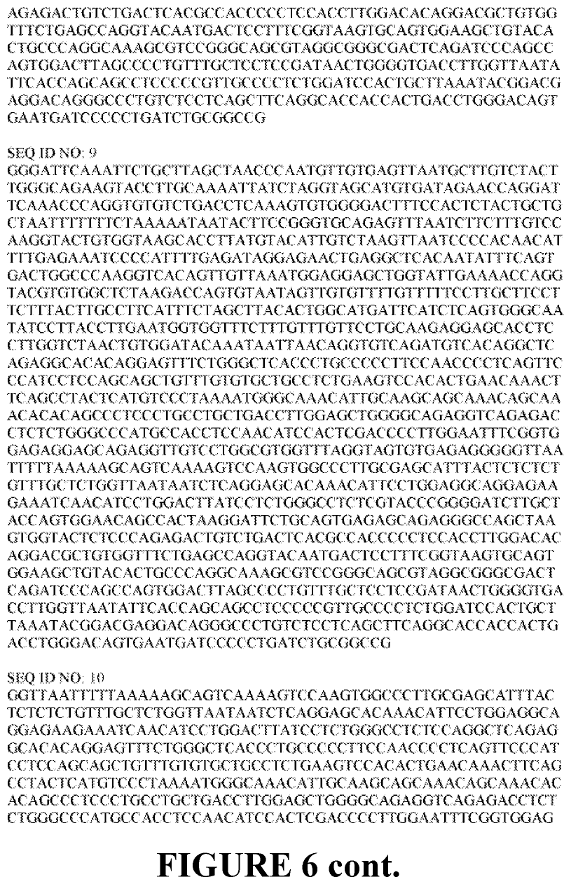

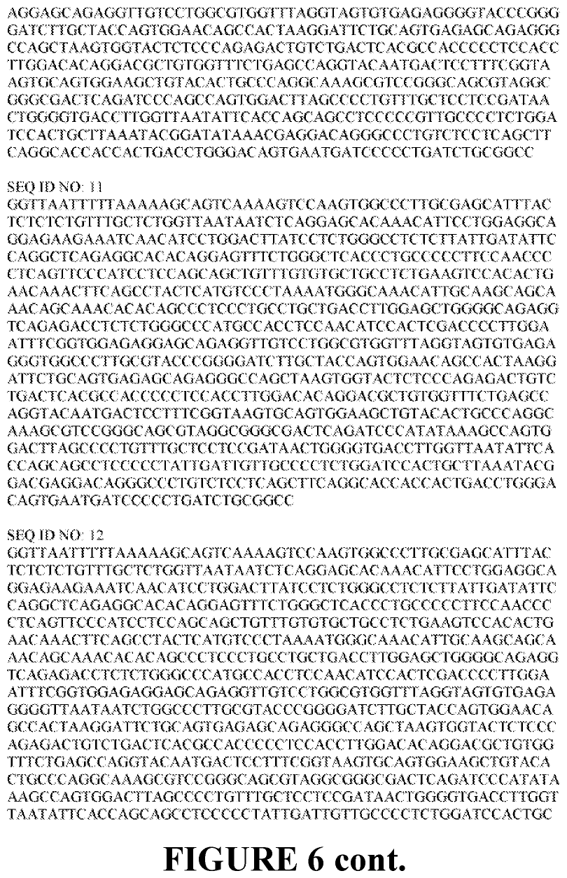

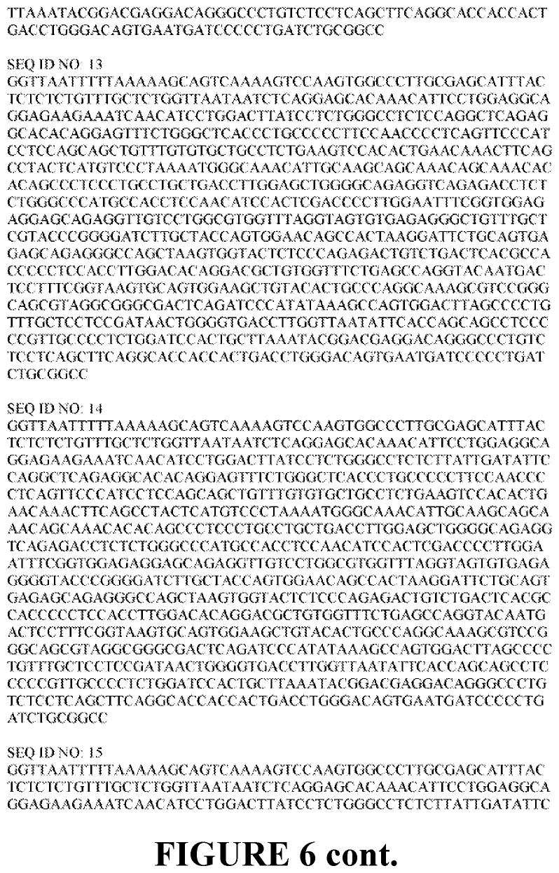

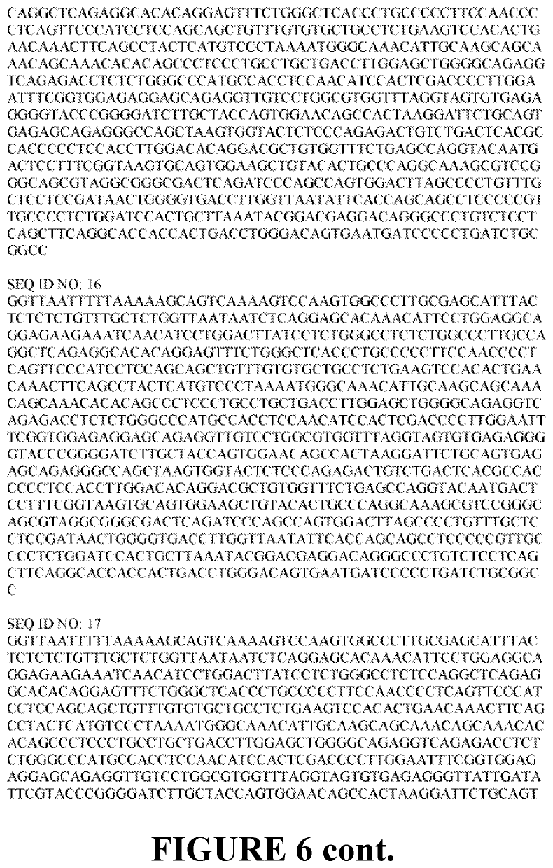

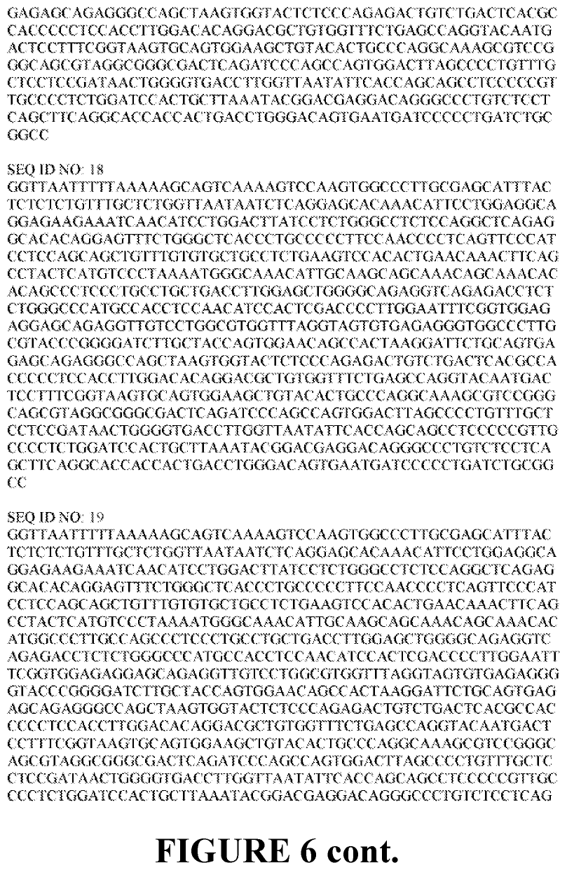

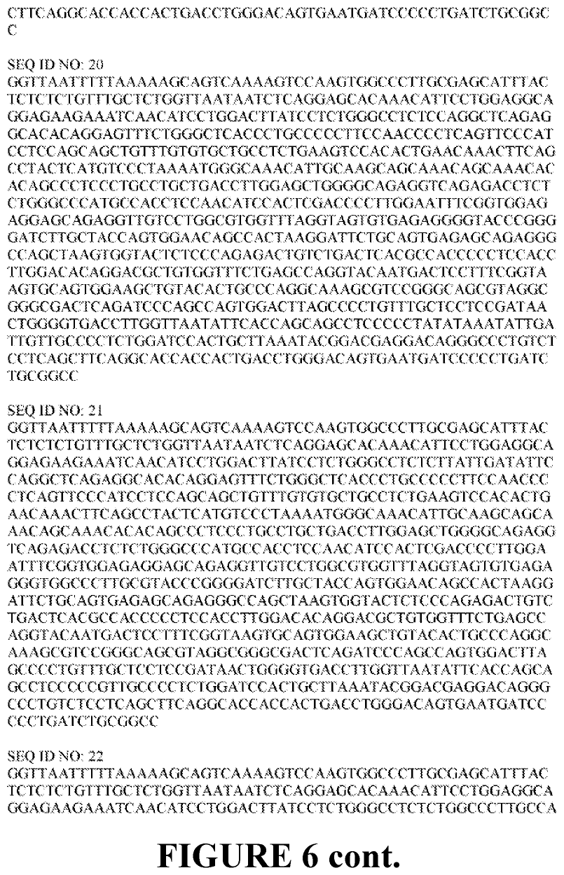

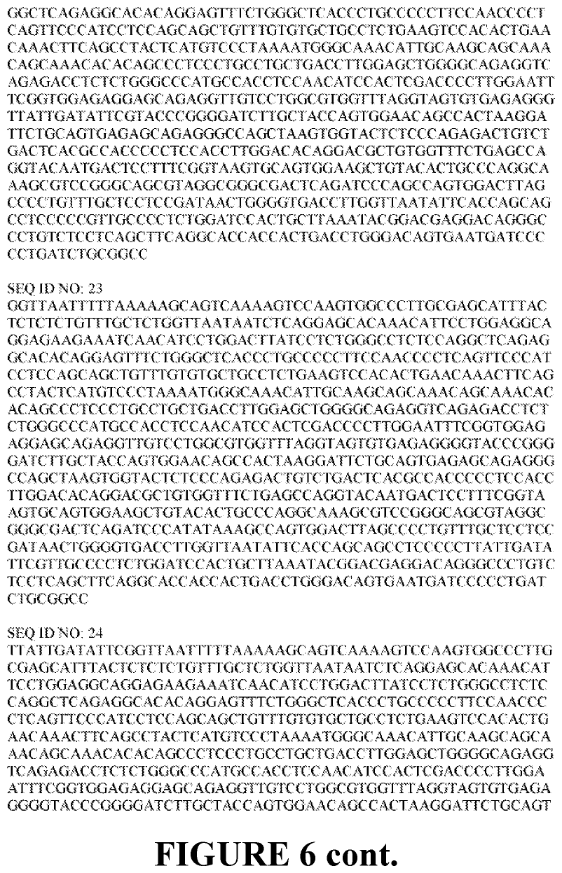

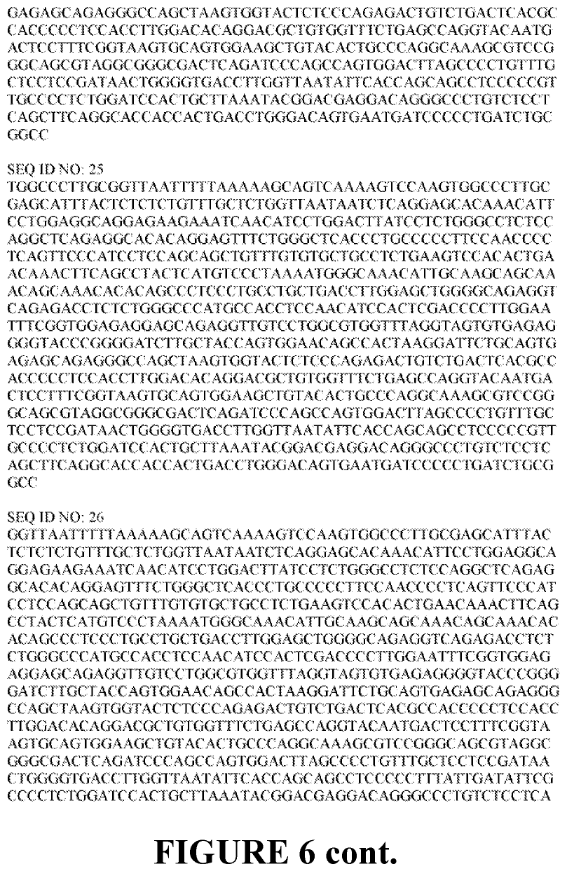

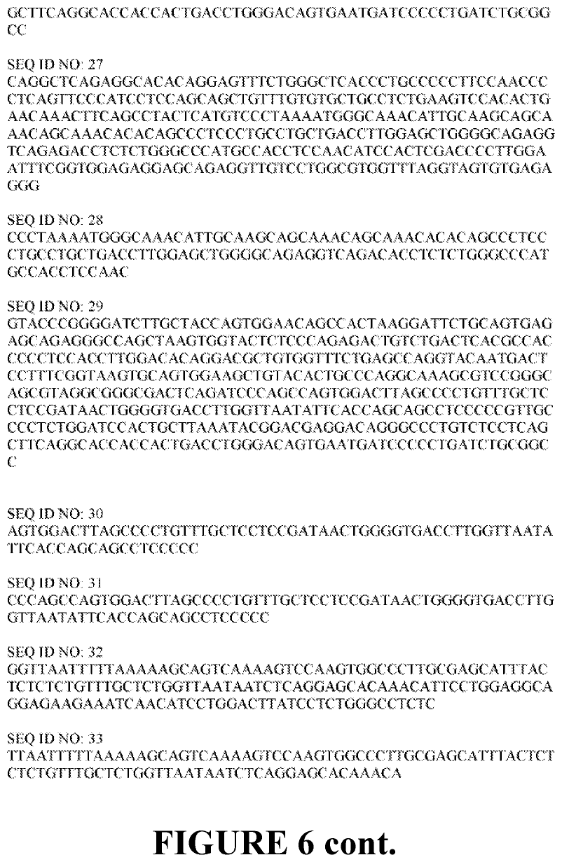

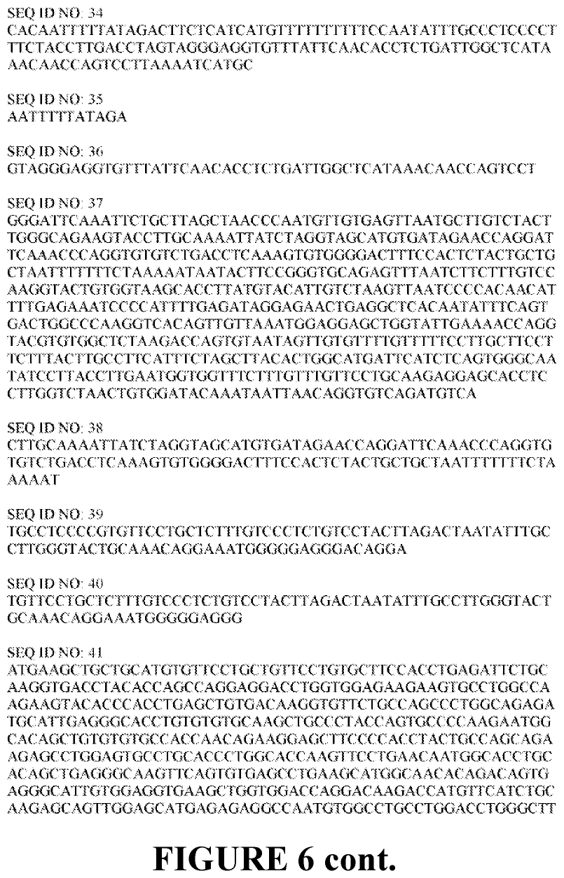

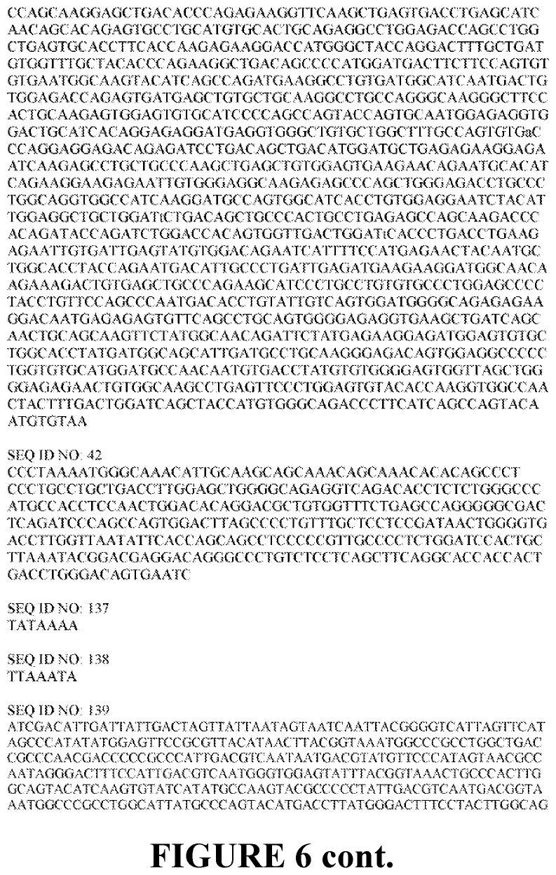

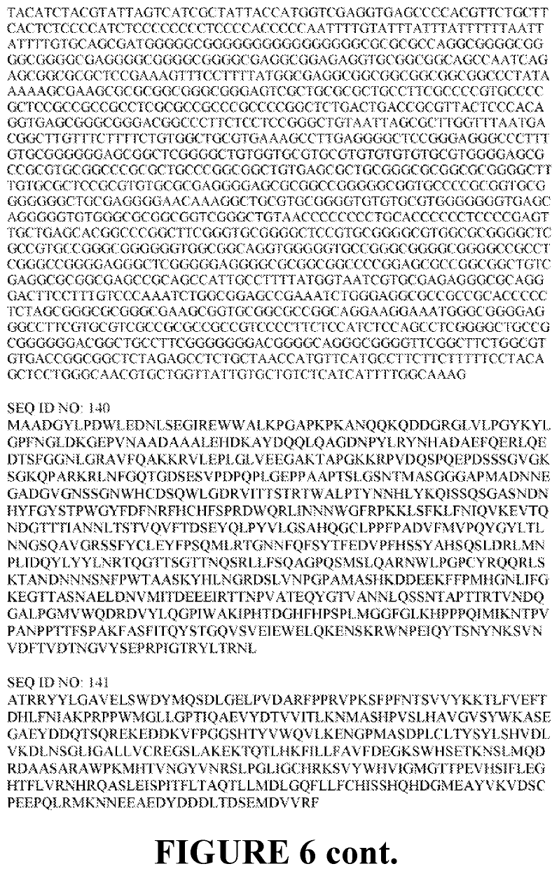

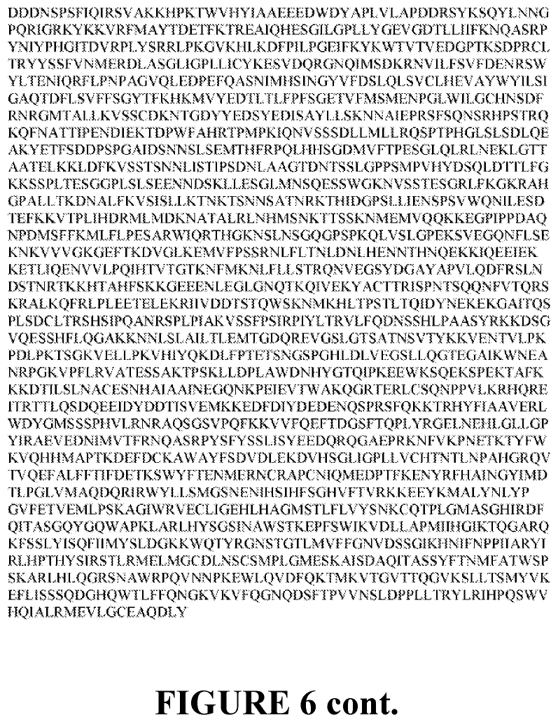

The CFI expression levels were normalised using luciferase expression as described above. Each expression cassette comprised wild-type human CFI and the BgpA poly A sequence. That data is presented in FIGS. 3-5. All of the novel hAAT-based TREs promoted greater expression than all of HCR-hAAT, HLP2, and FRE72.

### Example 2—Liver Specificity Study of Candidate Promoters

To determine whether the candidate promoters promoted expression of a transgene at a higher level in liver cells compared to cells from other tissues, specifically Huh7 (hepatocellular carcinoma), HEK293T (Kidney), 697 (B cell leukemia (early B cell)), BxPC-3 (Pancreas (adenocarcinoma), MCF7 (Breast (epithelial, adenocarcinoma), 1643 (Neuroblastoma), MRC-9 (Normal lung fibroblast), and AC-16 (cardiomyocyte) cells, were transduced with vectors comprising novel hAAT-based TREs NP1, NP2, NP6, and NP7 operably linked to GFP, and expression of GFP was measured.

### Methods

AAV vectors were generated which comprise an expression cassette which comprises one of NP1 (SEQ ID NO: 3), NP2 (SEQ ID NO: 4), NP6 (SEQ ID NO: 8), NP7 (SEQ ID NO: 9), or CAG (SEQ ID NO: 139) operably linked to GFP.

Cell lines were grown in either DMEM(-F12), IMDM, RPMI or EMEM supplemented with FBS (as set out in Table 2 below). For all cell lines 2×10⁴ cells were transduced at MOI of 1×10⁵. All transductions were performed in duplicate. Suspension cells were counted and transduced in serum free media in 48-well plates (300 μL). Adherent cells were counted and plated in 96-well plates. Cells were allowed to adhere for 24 hours before transduction. The transduction mixes comprising the AAV vectors were prepared in X-VIVO media (Lonza) (50 μL) and added to cells. After 3 hours, the wells were topped up with 100 μL of cell line specific culture medium (table 2). After 24 hours, the transduction media was changed to culture media for all cell lines. After 4 days, the cells were lifted with 40 μL TrypLE and 110 μL of PBS were added. GFP expression was assessed by FACS using 110 μL of cell suspension and gating on single cells to record at least 2000 events per replicate (BD LSR Fortessa X-20). Gating parameters for each cell line were set for the negative control (untransduced, control) to select all cells that were brighter than the control population. Additionally, Mean Intensity Fluorescence (MFI) values were obtained for all single cells for each sample. GFP fluorescence was analysed in the FITC channel using a blue laser at 488 nm.

### Results

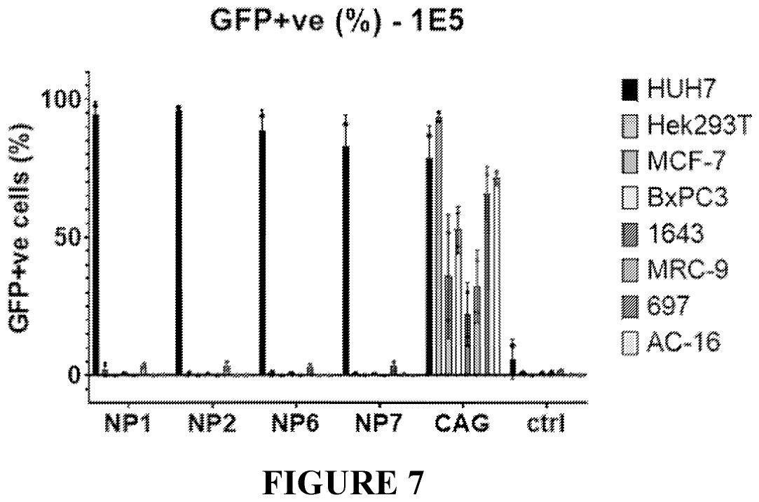

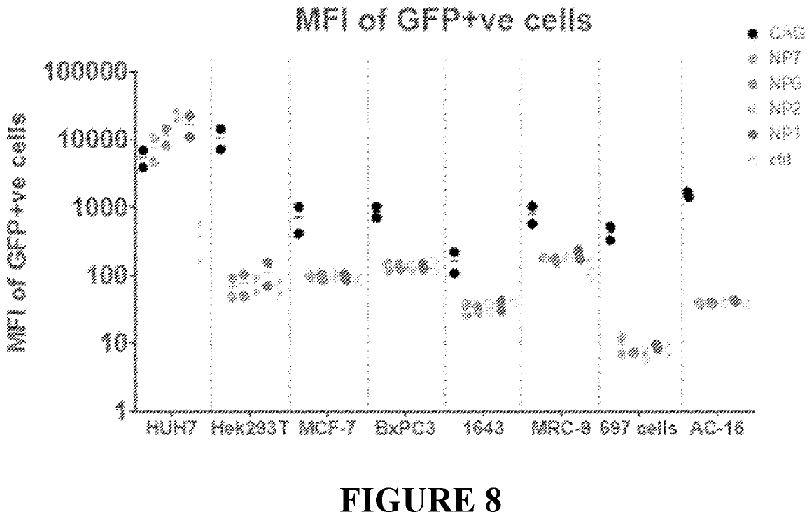

The percentage GFP positivity and MFI was measured for each of the cell lines transduced with vectors comprising NP1, NP2, NP6, NP7, or CAG. These data are presented in FIGS. 7 and 8. The control shows background GFP levels from cells which were not transduced. All of the novel hAAT-based promoters show strong liver specificity when compared with the CAG promoter.

TABLE 2

| Cell | | | Culture medium |
|---|---|---|---|
| line | Origin | Growth | (+10% FBS) |
| HUH7 | Hepatocellular carcinoma | Adherent | DMEM low gluc. + |
| | (Liver) | | glutamax |
| HEK293T | Kidney | Adherent | DMEM |
| 697 | B cell leukemia (early B | Suspension | RPMI (suspension) |
| | cell) | | |
| BxPC-3 | Pancreas | Adherent | RPMI |
| | (adenocarcinoma) | | |
| MCF7 | Breast (epithelial; | Adherent | DMEM |
| | adenocarcinoma) | | |
| 1643 | Neuroblastoma | Adherent | IMDM + ITS + |
| | | | glutamax + 20% |
| | | | FBS |
| MRC-9 | Normal lung fibroblast | Adherent | EMEM |
| | (embryo) | | |
| AC-16 | Cardiomyocytes | Adherent | DMEM-F12 + |
| | | | 12.5% FBS |

### Example 3—Comparison of GLA Expression in Huh7 Cells In Vitro when the Transgene is Under the Control of Different TREs

### Production of Recombinant AAV Vectors

AAV vectors were made by co-transfection of adherent HEK293 T-cells with a combination of plasmids consisting of the vector plasmid in which an alpha-galactosidase (GLA) transgene was under the control of one of the HCR-hAAT, NP1, NP2, NP5, and NP6 TREs, an adenoviral helper plasmid, and a packaging plasmid in which the AAV cap gene (SEQ ID NO: 140) was downstream of AAV2 Rep gene under the control of the endogenous TREs. Vectors were purified using AVB column chromatography. Quantitation of all vectors was performed by qPCR assay as well as alkaline gel analysis.

### Transduction of Huh-7 Cells

Huh-7 cells were maintained in DMEM low glucose supplemented with 10% FBS and 1% GlutaMAX (D-10). Cells were counted by Countess™ II FL Automated Cell Counter. For transduction, cells were seeded at 2×10⁵ cells/well in a 12-well plate and allowed to adhere for 24 hours prior to transduction. Subsequently, to increase the transduction efficacy in vitro, cells were treated with Mitomycin C (10 μg/mL in D-10) for one hour at 37° C. and washed with DMEM low glucose supplemented with 1% GlutaMAX (D-10). The transduction mix of D-10 containing AAV viral particles was prepared, added to the cells (500 μL/well) and incubated overnight (for approximately 16 hours) at 37° C., 5% CO₂. Transduction occurred at the required AAV multiplicity of infection (MOI) 1×10⁵ vg/cell. Each transduction was performed in triplicate.

### GLA Activity Assay (4-MUG Assay)

The activity of secreted GLA in the supernatant was measured at day 3 post-transduction by using a fluorescence end-point assay developed for clinical analysis. The hydrolysis of the substrate 4-methylumbelliferyl-a-D-galactopyranoside to the products 4-methylumbelliferone (4-MUG) and galactose by GLA can be measured via fluorescence of 4-MUG after incubation at 37° C. For this reaction 20 μl of cell culture supernatant were mixed with 20 μl reaction mix (comprising 3.38 mg/ml 4-methylumbelliferyl-a-D-galactopyranoside and 44.2 mg/ml N-acetyl-D-galactosamine in 0.5M acetate pH4.8), incubated at 37ºC for 2h and stopped by adding 200 μl IM glycin buffer at pH10.4. Activity of GLA was quantified by monitoring levels of the product on the Spectramax i3x plate reader at 365 nm excitation and emission at 450 nm. Through the addition of known standards and blanks (see Table 3 below) sample plasma+GLA activity can be calculated as nmol/hr/mL for each sample according to the formula below.

$$GLA Activity ( \frac{\frac{n mol}{hour}}{mL} ) = \frac{( T - B ) \times 60 \times 1000}{S \times 120 \times 20}$$

wherein
S=RFU(Standard)−RFU(Standard Blank)=Fluorescence of 1 nmol 4-MUG
T=average RFU reading in the test wells
B=RFU(Substrate Blank) for each sample

TABLE 3

| Standard/Blank | Composition |
|---|---|
| Standard | 20 μL sample, 200 μL stopping solution, |
| | 20 μL 50 μM 4-methylumbelliferyl-a-D- |
| | galactopyranoside solution |
| Standard Blank | 20 μL sample, 200 μL stopping solution, |
| | 20 μL water |
| Substrate Blank | 20 μL sample, 200 μL stopping solution, |
| | 20 μL reaction mix (added post incubation) |

### Results

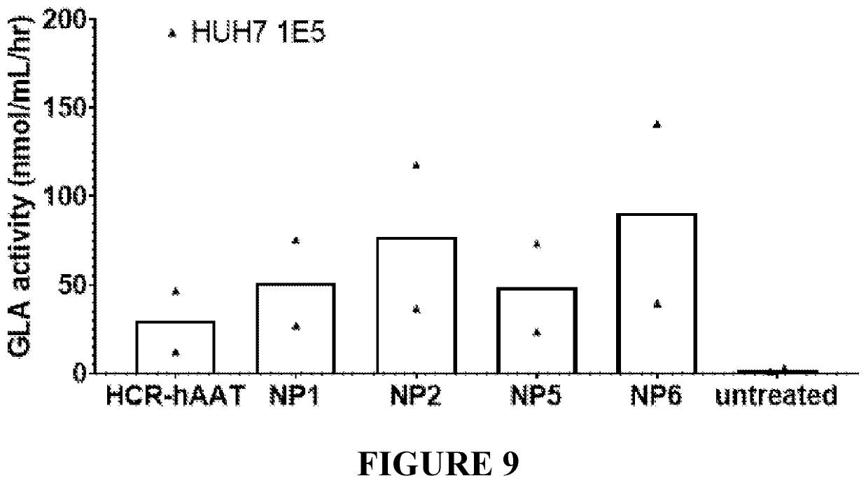

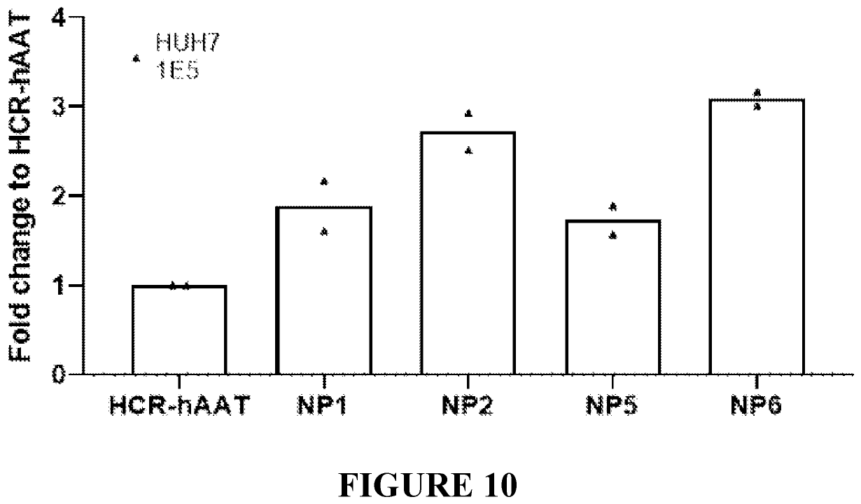

The GLA activity was measured for each of the cell lines transduced with vectors comprising HCR-hAAT, NP1, NP2, NP5, and NP6. These data are presented in FIGS. 9 and 10. All of the novel hAAT-based TREs show increased GLA activity compared to HCR-hAAT.

### Embodiments of the Invention

1. A transcription regulatory element (TRE) that:
(a) promotes greater expression than HCR-hAAT:
(b) promotes greater expression than FRE72:
(c) comprises a hAAT sequence element; and/or
(d) comprises an AMBP sequence element.

2. The TRE of embodiment 1, wherein the TRE is greater than 500, greater than 600, greater than 700, greater than 800, greater than 900, greater than 1000, greater than 1100, greater than 1200, greater than 1300, greater than 1400, greater than 1500, or greater than 1600 nucleotides in length.

3. The TRE of embodiment 1 or 2, wherein the TRE is less than 2000, less than 1800, less than 1600, less than 1500, less than 1400, less than 1300, less than 1200, less than 1100, less than 1000, less than 900, less than 800, less than 700, or less than 600 nucleotides in length.

4. The TRE of any one of the preceding embodiments, wherein the TRE is between 400 and 2000, between 500 and 1800, between 600 and 1400, between 800 and 1600, between 500 and 600, between 600 and 700, between 700 and 800, between 800 and 900, between 900 and 1000, between 1000 and 1100, between 1100 and 1200, between 1200 and 1300, between 1300 and 1400, between 1400 and 1500, between 1500 and 1600, or between 1600 and 1700 nucleotides in length.

5. The TRE of any one of the preceding embodiments, wherein the TRE comprises a hAAT sequence element.

6. The TRE of any one of the preceding embodiments, wherein the hAAT sequence element comprises a nucleotide sequence of SEQ ID NO: 30 or a variant of SEQ ID NO: 30 that differs by 1, 2, or 3 nucleotides.

7. The TRE of any one of the preceding embodiments, wherein the hAAT sequence element comprises a nucleotide sequence of SEQ ID NO: 30.

8. The TRE of any one of the preceding embodiments, wherein the hAAT sequence element comprises a nucleotide sequence of SEQ ID NO: 31 or a variant of SEQ ID NO: 31 that differs by 1, 2, or 3 nucleotides.

9. The TRE of any one of the preceding embodiments, wherein the hAAT sequence element comprises a nucleotide sequence of SEQ ID NO: 31.

10. The TRE of any one of the preceding embodiments, wherein the hAAT sequence element comprises a nucleotide sequence at least 80%, at least 85%, at least 90%, at least 95%, at least 98%, or at least 99% identical to SEQ ID NO: 29 or a fragment of SEQ ID NO: 29 that is at least 200, at least 300, or at least 400 nucleotides long.

11. The TRE of any one of the preceding embodiments, wherein the hAAT sequence element comprises a nucleotide sequence at least 98% identical to SEQ ID NO: 29.

12. The TRE of any one of the preceding embodiments, wherein the hAAT sequence element comprises a nucleotide sequence of SEQ ID NO: 29.

13. The TRE of any one of the preceding embodiments, wherein the TRE comprises a first and a second hAAT sequence element.

14. The TRE of embodiment 13, wherein the second hAAT sequence element is 5′ of the first hAAT sequence element and 5′ of the AMBP sequence element.

15. The TRE of embodiment 13 or embodiment 14, wherein the second hAAT sequence element comprises a nucleotide sequence of SEQ ID NO: 30 or a variant of SEQ ID NO: 30 that differs by 1, 2, or 3 nucleotides.

16. The TRE of embodiment 15, wherein the second hAAT sequence element comprises a nucleotide sequence of SEQ ID NO: 30.

17. The TRE of any one of embodiments 1-12, wherein the TRE does not comprise more than one hAAT sequence element.

18. The TRE of any one of the preceding embodiments, wherein the TRE comprises an AMBP sequence element.

19. The TRE of any one of the preceding embodiments, wherein the TRE comprising the AMBP sequence element promotes equivalent or greater expression than a corresponding TRE lacking an AMBP sequence element.

20. The TRE of any one of the preceding embodiments, wherein the TRE comprising the AMBP sequence element promotes greater expression than a corresponding TRE lacking an AMBP sequence element.

21. The TRE of any one of the preceding embodiments, wherein the AMBP sequence element comprises at least one binding site selected from the group consisting of an HNF1-1 binding site, an HNF4 binding site, an HNF3a binding site, an HNF1-2 binding site, and an HNF3-2 binding site.

22. The TRE of embodiment 21, wherein the AMBP sequence element comprises an HNF1-1 binding site, an HNF4 binding site, an HNF3a binding site, an HNF1-2 binding site, and an HNF3-2 binding site.

23. The TRE of any one of the preceding embodiments, wherein the AMBP sequence element comprises a nucleotide sequence of SEQ ID NO: 33 or a variant of SEQ ID NO: 33 that differs by 1, 2, or 3 nucleotides.

24. The TRE of any one of the preceding embodiments, wherein the AMBP sequence element comprises a nucleotide sequence of SEQ ID NO: 33.

25. The TRE of any one of the preceding embodiments, wherein the AMBP sequence element comprises a nucleotide sequence of SEQ ID NO: 32 or a variant of SEQ ID NO: 32 that differs by 1, 2, or 3 nucleotides.

26. The TRE of any one of the preceding embodiments, wherein the AMBP sequence element comprises a nucleotide sequence of SEQ ID NO: 32.

27. The TRE of any one of the preceding embodiments, wherein the AMBP sequence element is 5′ of the hAAT sequence element.

28. The TRE of any one of the preceding embodiments, wherein the TRE is liver specific.

29. The TRE of any one of the preceding embodiments, wherein the TRE does not comprise more than one AMBP sequence element.

30. The TRE of any one of the preceding embodiments, wherein the TRE promotes greater expression than HCR-hAAT.

31. The TRE of any one of the preceding embodiments, wherein the TRE promotes greater expression than FRE72.

32. The TRE of any one of the preceding embodiments, wherein the TRE promotes expression at least 1.2 fold, at least 1.5 fold, at least 1.8 fold, or at least 2 fold higher compared to HCR-hAAT.

33. The TRE of any one of the preceding embodiments, wherein the TRE promotes expression at least 1.6 fold or at least 1.8 fold higher compared to HCR-hAAT.

34. The TRE of any one of the preceding embodiments, wherein the TRE promotes expression at least 1.2 fold, at least 1.5 fold, at least 1.8 fold, or at least 2 fold higher compared to FRE72.

35. The TRE of any one of the preceding embodiments, wherein the TRE promotes expression at least 1.6 fold or at least 1.8 fold higher compared to FRE72.

36. The TRE of any one of the preceding embodiments, wherein expression is measured by an expression assay comprising:
(i) transfecting human hepatocytes with a plasmid comprising an expression cassette comprising a transgene operably linked to the TRE;
(ii) incubating the transgene under conditions suitable for transgene expression to occur; and
(iii) measuring the level of the transgene by ELISA using an antibody specific for a protein encoded by the transgene.

37. The TRE of embodiment 36, wherein the human hepatocytes are Huh7 cells.

38. The TRE of embodiment 36 or 37, wherein the transgene encodes CFI (Complement Factor I).

39. The TRE of any one of embodiments 36-38, wherein step (i) of transfecting human hepatocytes comprises co-transfecting the human hepatocytes with a plasmid comprising an expression cassette comprising a transgene encoding luciferase operably linked to a promoter and the expression assay further comprises measuring the total luciferase expression.

40. The TRE of any one of embodiments 1-35, wherein expression is measured by an activity assay comprising:
(i) transducing human hepatocytes with an AAV vector comprising an expression cassette comprising a transgene operably linked to the TRE;
(ii) incubating the transduced cells in conditions suitable for transgene expression to occur; and
(iii) measuring the level of the transgene by its activity.

41. The TRE of embodiment 40, wherein the human hepatocytes are Huh7 cells.

42. The TRE of embodiment 40 or 41, wherein the transgene encodes GLA (alpha-galactosidase).

43. The TRE of any one of embodiments 40-42, wherein the activity assay is a GLA activity assay.

44. The TRE of any one of the preceding embodiments, wherein the TRE comprises an ApoE-HCR1 sequence element.

45. The TRE of embodiment 44, wherein the TRE comprising the ApoE-HCR1 sequence element promotes equivalent or greater expression than a corresponding TRE lacking the ApoE-HCR1 sequence element.

46. The TRE of embodiment 44 or 45, wherein the TRE comprising the ApoE-HCR1 sequence element promotes greater expression than a corresponding TRE lacking the ApoE-HCR1 sequence element.

47. The TRE of any one of embodiments 44-46, wherein the ApoE-HCR1 sequence element comprises a nucleotide sequence of SEQ ID NO: 28 or a variant of SEQ ID NO: 28 that differs by 1, 2, or 3 nucleotides.

48. The TRE of any one of embodiments 44-47, wherein the ApoE-HCR1 sequence element comprises a nucleotide sequence of SEQ ID NO: 28.

49. The TRE of any one of embodiments 44-48, wherein the ApoE-HCR1 sequence element comprises a nucleotide sequence of SEQ ID NO: 27 or a variant of SEQ ID NO: 27 that differs by 1, 2, or 3 nucleotides.

50. The TRE of any one of embodiments 44-49, wherein the ApoE-HCR1 sequence element is 5′ of the hAAT sequence element.

51. The TRE of any one of embodiments 44-50, wherein the ApoE-HCR1 sequence element is 5′ of the AMBP sequence element.

52. The TRE of any one of embodiments 44-50, wherein the ApoE-HCR1 sequence element is 3′ of the AMBP sequence element.

53. The TRE of any one of embodiments 44-51, wherein the ApoE-HCR1 sequence element is 5′ of both the hAAT sequence element and the AMBP sequence element.

54. The TRE of any one of embodiments 44-50 or 52, wherein the ApoE-HCR1 sequence element is 5′ of the hAAT sequence element and 3′ of the AMBP sequence element.

55. The TRE of any one of the preceding embodiments, wherein the TRE does not comprise more than one ApoE-HCR1 sequence element.

56. The TRE of any one of the preceding embodiments, wherein the TRE comprises a CRM6 sequence element.

57. The TRE of embodiment 56, wherein the TRE comprising the CRM6 sequence element promotes equivalent or greater expression than a corresponding TRE lacking the CRM6 sequence element.

58. The TRE of embodiment 56 or embodiment 57, wherein the TRE comprising the CRM6 sequence element promotes equivalent expression than a corresponding TRE lacking the CRM6 sequence element.

59. The TRE of any one of embodiments 56-58, wherein the CRM6 sequence element comprises a binding site selected from the group consisting of an Sp1 binding site, an Sp2 binding site, an HNF3a binding site, and an HNF-1 binding site.

60. The TRE of embodiment 59, wherein the CRM6 sequence element comprises an Sp1 binding site, an Sp2 binding site, an HNF3a binding site, and an HNF1 binding site.

61. The TRE of any one of embodiments 56-60, wherein the CRM6 sequence element comprises a nucleotide sequence of SEQ ID NO: 35 or a variant of SEQ ID NO: 35 that differs by 1 or 2 nucleotides.

62. The TRE of any one of embodiments 56-61, wherein the CRM6 sequence element comprises a nucleotide sequence of SEQ ID NO: 35.

63. The TRE of any one of embodiments 56-62, wherein the CRM6 sequence element comprises a nucleotide sequence of SEQ ID NO: 36 or a variant of SEQ ID NO: 36 that differs by 1, 2, or 3 nucleotides.

64. The TRE of any one of embodiments 56-63, wherein the CRM6 sequence element comprises a nucleotide sequence of SEQ ID NO: 36.

65. The TRE of any one of embodiments 56-64, wherein the CRM6 sequence element comprises a nucleotide sequence of SEQ ID NO: 35 and a nucleotide sequence of SEQ ID NO: 36.

66. The TRE of any one of embodiments 56-65, wherein the CRM6 sequence element comprises a nucleotide sequence of SEQ ID NO: 34 or a variant of SEQ ID NO: 34 that differs by 1, 2, or 3 nucleotides.

67. The TRE of any one of embodiments 56-66, wherein the CRM6 sequence element comprises a nucleotide sequence of SEQ ID NO: 34.

68. The TRE of any one of embodiments 56-67, wherein the CRM6 sequence element is 5′ of the hAAT sequence element.

69. The TRE of any one of embodiments 56-68, wherein the CRM6 sequence element is 5′ of the AMBP sequence element.

70. The TRE of any one of embodiments 56-68, wherein the CRM6 sequence element is 3′ of the AMBP sequence element.

71. The TRE of any one of embodiments 56-69, wherein the CRM6 sequence element is 5′ of both the hAAT sequence element and the AMBP sequence element.

72. The TRE of any one of embodiments 56-68 or 70, wherein the CRM6 sequence element is 5′ of the hAAT sequence element and 3′ of the AMBP sequence element.

73. The TRE of any one of the preceding embodiments, wherein the TRE does not comprise more than one CRM6 element.

74. The TRE of any one of the preceding embodiments, wherein the TRE comprises an ALDOB sequence element.

75. The TRE of embodiment 74, wherein the TRE comprising the ALDOB sequence element promotes equivalent or greater expression than a corresponding TRE lacking the ALDOB sequence element.

76. The TRE of any one of embodiment 74 or 75, wherein the TRE comprising the ALDOB sequence element promotes equivalent expression than a corresponding TRE lacking the ALDOB sequence element.

77. The TRE of any one of embodiments 74-76, wherein the ALDOB sequence element comprises at least one binding site selected from the group consisting of an SP1 binding site, an HNF1a binding site, a C/EBP binding site, a GATA2 binding site, a USF1 binding site, and a USF2 binding site.

78. The TRE of any one of embodiments 74-77, wherein the ALDOB sequence element comprises an SP1 binding site, an HNF1a binding site, a C/EBP binding site, a GATA2 binding site, a USF1 binding site, and a USF2 binding site.

79. The TRE of any one of embodiments 74-78, wherein the ALDOB sequence element comprises a nucleotide sequence of SEQ ID NO: 38 or a variant of SEQ ID NO: 38 that differs by 1, 2, or 3 nucleotides.

80. The TRE of any one of embodiments 74-79, wherein the ALDOB sequence element comprises a nucleotide sequence of SEQ ID NO: 38.

81. The TRE of any one of embodiments 74-80, wherein the ALDOB sequence element comprises a nucleotide sequence of SEQ ID NO: 37 or a variant of SEQ ID NO: 37 that differs by 1, 2, or 3 nucleotides.

82. The TRE of embodiment 81, wherein the ALDOB sequence element comprises a nucleotide sequence of SEQ ID NO: 37.

83. The TRE of any one of embodiments 74-82, wherein the ALDOB sequence element is 5′ of the hAAT sequence element.

84. The TRE of any one of embodiments 74-83, wherein the ALDOB sequence element is 5′ of the AMBP sequence element.

85. The TRE of any one of embodiments 74-83, wherein the ALDOB sequence element is 3′ of the AMBP sequence element.

86. The TRE of any one of embodiments 74-84, wherein the ALDOB sequence element is 5′ of both the hAAT sequence element and the AMBP sequence element.

87. The TRE of any one of embodiments 74-83 or 85, wherein the ALDOB sequence element is 5′ of the hAAT sequence element and 3′ of the AMBP sequence element.

88. The TRE of any one of the preceding embodiments, wherein the TRE does not comprise more than one ALDOB element.

89. The TRE of any one of the preceding embodiments, wherein the TRE comprises an F2 sequence element.

90. The TRE of embodiment 89, wherein the TRE comprising the F2 sequence element promotes equivalent or greater expression than a corresponding TRE lacking the F2 sequence element.

91. The TRE of embodiment 89 or embodiment 90, wherein the TRE comprising the F2 sequence element promotes greater expression than a corresponding TRE lacking the F2 sequence element.

92. The TRE of any one of embodiments 89-91, wherein the F2 sequence element comprises at least one binding site selected from the group consisting of an HNF4a binding site, an HNF3a binding site, an HNF1a binding site, an HNF3b binding site, an STAT3 binding site, an RBPJ binding site, and an SP3 binding site.

93. The TRE of embodiment 92, wherein the F2 sequence element comprises an HNF4a binding site, an HNF3a binding site, an HNF1a binding site, an HNF3b binding site, an STAT3 binding site, an RBPJ binding site, and an SP3 binding site.

94. The TRE of any one of embodiments 89-93, wherein the F2 sequence element comprises a nucleotide sequence of SEQ ID NO: 40 or a variant of SEQ ID NO: 40 that differs by 1, 2, or 3 nucleotides.

95. The TRE of embodiment 94, wherein the F2 sequence element comprises a sequence of SEQ ID NO: 40.

96. The TRE of any one of embodiments 89-95, wherein the F2 sequence element comprises a nucleotide sequence of SEQ ID NO: 39 or a variant of SEQ ID NO: 39 that differs by 1, 2, or 3 nucleotides.

97. The TRE of embodiment 96, wherein the F2 sequence element comprises a nucleotide sequence of SEQ ID NO: 39.

98. The TRE of any one of embodiments 89-97, wherein the F2 sequence element is 5′ of the hAAT sequence element.

99. The TRE of any one of embodiments 89-98, wherein the F2 sequence element is 5′ of the AMBP sequence element.

100. The TRE of any one of embodiments 89-98, wherein the F2 sequence element is 3′ of the AMBP sequence element.

101. The TRE of any one of embodiments 89-99, wherein the F2 sequence element is 5′ of both the hAAT sequence element and the AMBP sequence element.

102. The TRE of any one of embodiments 89-98 or 100, wherein the F2 sequence element is 5′ of the hAAT sequence element and 3′ of the AMBP sequence element.

103. The TRE of any one of the preceding embodiments, wherein the TRE does not comprise more than one F2 sequence elements.

104. The TRE of any one of the preceding embodiments, wherein the TRE comprises:
(a) a nucleotide sequence having at least 80%, at least 85%, at least 90%, at least 95%, at least 98%, or at least 99% sequence identity to any one of SEQ ID NO: 3-26; or
(b) a nucleotide sequence of any one of SEQ ID NO: 3-26.

105. The TRE of any one of the preceding embodiments, wherein the TRE comprises:
(a) a nucleotide sequence having at least 80%, at least 85%, at least 90%, at least 95%, at least 98%, or at least 99% sequence identity to SEQ ID NO: 5; or
(b) a nucleotide sequence of SEQ ID NO: 5.

106. The TRE of any one of embodiments 1-104, wherein the TRE comprises:
(a) a nucleotide sequence having at least 80%, at least 85%, at least 90%, at least 95%, at least 98%, or at least 99% sequence identity to SEQ ID NO: 9; or
(b) a nucleotide sequence of SEQ ID NO: 9.

107. The TRE of any one of the preceding embodiments, wherein the TRE further comprises:
(a) a TATA box:
(b) an HNF6 binding site:
(c) an HNF4a binding site:
(d) an HNF1 binding site; and/or
(e) an HNF3 binding site.

108. The TRE of embodiment 107, wherein the TRE further comprises a TATA box, optionally a canonical TATA box.

109. The TRE of embodiment 108, wherein the TRE comprises a hAAT sequence element and the TATA box is inserted into the hAAT sequence element.

110. The TRE of embodiment 108 or 109, wherein the hAAT sequence element comprises a nucleotide sequence of SEQ ID NO: 30 or a variant of SEQ ID NO: 30 that differs by 1, 2, or 3 nucleotides and the TATA box is 3′ of the nucleotide sequence of SEQ ID NO: 30 or variant of SEQ ID NO: 30.

111. The TRE of embodiment 108 or 109, wherein the hAAT sequence element comprises a nucleotide sequence of SEQ ID NO: 30 or a variant of SEQ ID NO: 30 that differs by 1, 2, or 3 nucleotides and the TATA box is 5′ of the nucleotide sequence of SEQ ID NO: 30 or variant of SEQ ID NO: 30.

112. The TRE of any one of embodiments 107-111, wherein the TRE promotes equivalent or greater expression than a corresponding TRE not comprising the TATA box.

113. The TRE of any one of embodiments 107-112, wherein the TRE further comprises an HNF6 binding site.

114. The TRE of embodiment 113, wherein the TRE comprises an HNF6 binding site, and:
(a) the TRE comprises a hAAT sequence element that comprises a nucleotide sequence of SEQ ID NO: 30 or a variant of SEQ ID NO: 30 that differs by 1, 2, or 3 nucleotides and the HNF6 binding site is 5′ of the hAAT sequence element that comprises a nucleotide sequence of SEQ ID NO: 30 or a variant of SEQ ID NO: 30 that differs by 1, 2, or 3 nucleotides; and/or
(b) the TRE comprises a hAAT sequence element that comprises a nucleotide sequence of SEQ ID NO: 31 or a variant of SEQ ID NO: 31 that differs by 1, 2, or 3 nucleotides and the HNF6 binding site is 5′ of the hAAT sequence element that comprises a nucleotide sequence of SEQ ID NO: 31 or a variant of SEQ ID NO: 31 that differs by 1, 2, or 3 nucleotides; and/or
(c) the TRE comprises a hAAT sequence element that comprises a nucleotide sequence of SEQ ID NO: 29 or a variant of SEQ ID NO: 29 that differs by 1, 2, or 3 nucleotides and the HNF6 binding site is 5′ of the hAAT sequence element that comprises a nucleotide sequence of SEQ ID NO: 29 or a variant of SEQ ID NO: 29 that differs by 1, 2, or 3 nucleotides; and/or
(d) the TRE comprises a hAAT sequence element that comprises a nucleotide sequence of SEQ ID NO: 30 or a variant of SEQ ID NO: 30 that differs by 1, 2, or 3 nucleotides and the HNF6 binding site is 3′ of the hAAT sequence element that comprises a nucleotide sequence of SEQ ID NO: 30 or a variant of SEQ ID NO: 30 that differs by 1, 2, or 3 nucleotides; and/or
(e) the TRE comprises a hAAT sequence element that comprises a nucleotide sequence of SEQ ID NO: 31 or a variant of SEQ ID NO: 31 that differs by 1, 2, or 3 nucleotides and the HNF6 binding site is 3′ of the hAAT sequence element that comprises a nucleotide sequence of SEQ ID NO: 31 or a variant of SEQ ID NO: 31 that differs by 1, 2, or 3 nucleotides; and/or
(f) the TRE comprises a hAAT sequence element that comprises a nucleotide sequence of SEQ ID NO: 29 or a variant of SEQ ID NO: 29 that differs by 1, 2, or 3 nucleotides and the HNF6 binding site is 3′ of the hAAT sequence element that comprises a nucleotide sequence of SEQ ID NO: 29 or a variant of SEQ ID NO: 29 that differs by 1, 2, or 3 nucleotides; and/or
(g) the TRE comprises an ApoE-HCR1 sequence element that comprises a nucleotide sequence of SEQ ID NO: 28 or a variant of SEQ ID NO: 28 that differs by 1, 2, or 3 nucleotides and the HNF6 binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
(h) the TRE comprises an ApoE-HCR1 sequence element that comprises a nucleotide sequence of SEQ ID NO: 27 or a variant of SEQ ID NO: 27 that differs by 1, 2, or 3 nucleotides and the HNF6 binding site is 3′ of the ApoE-HCR1 sequence element that comprises a nucleotide sequence of SEQ ID NO: 27 or a variant of SEQ ID NO: 27 that differs by 1, 2, or 3 nucleotides; and/or
(i) the TRE comprises an ApoE-HCR1 sequence element that comprises a nucleotide sequence of SEQ ID NO: 28 or a variant of SEQ ID NO: 28 that differs by 1, 2, or 3 nucleotides and the HNF6 binding site is 5′ of the ApoE-HCR1 sequence element that comprises a nucleotide sequence of SEQ ID NO: 28 or a variant of SEQ ID NO: 28 that differs by 1, 2, or 3 nucleotides; and/or
(j) the TRE comprises an ApoE-HCR1 sequence element that comprises a nucleotide sequence of SEQ ID NO: 27 or a variant of SEQ ID NO: 27 that differs by 1, 2, or 3 nucleotides and the HNF6 binding site is 5′ of the ApoE-HCR1 sequence element that comprises a nucleotide sequence of SEQ ID NO: 27 or a variant of SEQ ID NO: 27 that differs by 1, 2, or 3 nucleotides; and/or
(k) the TRE comprises an AMBP sequence element that comprises a nucleotide sequence of SEQ ID NO: 33 or a variant of SEQ ID NO: 33 that differs by 1, 2, or 3 nucleotides and the HNF6 binding site is 3′ of the AMBP sequence element that comprises a nucleotide sequence of SEQ ID NO: 33 or a variant of SEQ ID NO: 33 that differs by 1, 2, or 3 nucleotides; and/or
(l) the TRE comprises an AMBP sequence element that comprises a nucleotide sequence of SEQ ID NO: 32 or a variant of SEQ ID NO: 32 that differs by 1, 2, or 3 nucleotides and the HNF6 binding site is 3′ of the AMBP sequence element that comprises a nucleotide sequence of SEQ ID NO: 32 or a variant of SEQ ID NO: 32 that differs by 1, 2, or 3 nucleotides; and/or
(m) the TRE comprises an AMBP sequence element that comprises a nucleotide sequence of SEQ ID NO: 33 or a variant of SEQ ID NO: 33 that differs by 1, 2, or 3 nucleotides and the HNF6 binding site is 5′ of the AMBP sequence element that comprises a nucleotide sequence of SEQ ID NO: 33 or a variant of SEQ ID NO: 33 that differs by 1, 2, or 3 nucleotides; and/or
(n) the TRE comprises an AMBP sequence element that comprises a nucleotide sequence of SEQ ID NO: 32 or a variant of SEQ ID NO: 32 that differs by 1, 2, or 3 nucleotides and the HNF6 binding site is 5′ of the AMBP sequence element that comprises a nucleotide sequence of SEQ ID NO: 32 or a variant of SEQ ID NO: 32 that differs by 1, 2, or 3 nucleotides.

115. The TRE of any one of embodiments 113-114, wherein the TRE promotes equivalent or greater expression than a corresponding TRE not comprising the HNF6 binding site.

116. The TRE of any one of embodiments 113-115, wherein the TRE comprises a HNF6 binding site and a TATA box and the TRE promotes equivalent or greater expression than a corresponding TRE not comprising the HNF6 binding site and the TATA box.

117. The TRE of any one of embodiments 107-116, wherein the TRE further comprises an HNF4a binding site.

118. The TRE of embodiment 117, wherein the TRE comprises an HNF4a binding site, and:
(a) the TRE comprises a hAAT sequence element that comprises a nucleotide sequence of SEQ ID NO: 30 or a variant of SEQ ID NO: 30 that differs by 1, 2, or 3 nucleotides and the HNF4a binding site is 5′ of the hAAT sequence element that comprises a nucleotide sequence of SEQ ID NO: 30 or a variant of SEQ ID NO: 30 that differs by 1, 2, or 3 nucleotides; and/or
(b) the TRE comprises a hAAT sequence element that comprises a nucleotide sequence of SEQ ID NO: 31 or a variant of SEQ ID NO: 31 that differs by 1, 2, or 3 nucleotides and the HNF4a binding site is 5′ of the hAAT sequence element that comprises a nucleotide sequence of SEQ ID NO: 31 or a variant of SEQ ID NO: 31 that differs by 1, 2, or 3 nucleotides; and/or
(c) the TRE comprises a hAAT sequence element that comprises a nucleotide sequence of SEQ ID NO: 29 or a variant of SEQ ID NO: 29 that differs by 1, 2, or 3 nucleotides and the HNF4a binding site is 5′ of the hAAT sequence element that comprises a nucleotide sequence of SEQ ID NO: 29 or a variant of SEQ ID NO: 29 that differs by 1, 2, or 3 nucleotides; and/or
(d) the TRE comprises an ApoE-HCR1 sequence element that comprises a nucleotide sequence of SEQ ID NO: 28 or a variant of SEQ ID NO: 28 that differs by 1, 2, or 3 nucleotides and the HNF4a binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
(e) the TRE comprises an ApoE-HCR1 sequence element that comprises a nucleotide sequence of SEQ ID NO: 28 or a variant of SEQ ID NO: 28 that differs by 1, 2, or 3 nucleotides and the HNF4a binding site is 5′ of the ApoE-HCR1 sequence element that comprises a nucleotide sequence of SEQ ID NO: 28 or a variant of SEQ ID NO: 28 that differs by 1, 2, or 3 nucleotides; and/or
(f) the TRE comprises an ApoE-HCR1 sequence element that comprises a nucleotide sequence of SEQ ID NO: 27 or a variant of SEQ ID NO: 27 that differs by 1, 2, or 3 nucleotides and the HNF4a binding site is 5′ of the ApoE-HCR1 sequence element that comprises a nucleotide sequence of SEQ ID NO: 27 or a variant of SEQ ID NO: 27 that differs by 1, 2, or 3 nucleotides; and/or
(g) the TRE comprises an AMBP sequence element that comprises a nucleotide sequence of SEQ ID NO: 33 or a variant of SEQ ID NO: 33 that differs by 1, 2, or 3 nucleotides and the HNF4a binding site is 3′ of the AMBP sequence element that comprises a nucleotide sequence of SEQ ID NO: 33 or a variant of SEQ ID NO: 33 that differs by 1, 2, or 3 nucleotides; and/or
(h) the TRE comprises an AMBP sequence element that comprises a nucleotide sequence of SEQ ID NO: 32 or a variant of SEQ ID NO: 32 that differs by 1, 2, or 3 nucleotides and the HNF4a binding site is 3′ of the AMBP sequence element that comprises a nucleotide sequence of SEQ ID NO: 32 or a variant of SEQ ID NO: 32 that differs by 1, 2, or 3 nucleotides; and/or
(i) the TRE comprises an AMBP sequence element that comprises a nucleotide sequence of SEQ ID NO: 33 or a variant of SEQ ID NO: 33 that differs by 1, 2, or 3 nucleotides and the HNF4a binding site is 5′ of the AMBP sequence element that comprises a nucleotide sequence of SEQ ID NO: 33 or a variant of SEQ ID NO: 33 that differs by 1, 2, or 3 nucleotides; and/or
(j) the TRE comprises an AMBP sequence element that comprises a nucleotide sequence of SEQ ID NO: 32 or a variant of SEQ ID NO: 32 that differs by 1, 2, or 3 nucleotides and the HNF4a binding site is 5′ of the AMBP sequence element that comprises a nucleotide sequence of SEQ ID NO: 32 or a variant of SEQ ID NO: 32 that differs by 1, 2, or 3 nucleotides.

119. The TRE of any one of embodiments 117-118, wherein the TRE promotes equivalent or greater expression than a corresponding TRE not comprising the HNF4a binding site.

120. The TRE of any one of embodiments 117-119, wherein the TRE comprises a HNF6 binding site and an HNF4a binding site and the TRE promotes equivalent or greater expression than a corresponding TRE not comprising the HNF6 binding site and the HNF4a binding site.

121. The TRE of any one of embodiments 107-120, wherein the TRE further comprises an HNF1 binding site.

122. The TRE of embodiment 121, wherein the TRE comprises an HNF1 binding site, and:
(a) the TRE comprises a hAAT sequence element that comprises a nucleotide sequence of SEQ ID NO: 30 or a variant of SEQ ID NO: 30 that differs by 1, 2, or 3 nucleotides and the HNF1 binding site is 5′ of the hAAT sequence element that comprises a nucleotide sequence of SEQ ID NO: 30 or a variant of SEQ ID NO: 30 that differs by 1, 2, or 3 nucleotides; and/or
(b) the TRE comprises a hAAT sequence element that comprises a nucleotide sequence of SEQ ID NO: 31 or a variant of SEQ ID NO: 31 that differs by 1, 2, or 3 nucleotides and the HNF1 binding site is 5′ of the hAAT sequence element that comprises a nucleotide sequence of SEQ ID NO: 31 or a variant of SEQ ID NO: 31 that differs by 1, 2, or 3 nucleotides; and/or
(c) the TRE comprises a hAAT sequence element that comprises a nucleotide sequence of SEQ ID NO: 29 or a variant of SEQ ID NO: 29 that differs by 1, 2, or 3 nucleotides and the HNF1 binding site is 5′ of the hAAT sequence element that comprises a nucleotide sequence of SEQ ID NO: 29 or a variant of SEQ ID NO: 29 that differs by 1, 2, or 3 nucleotides; and/or
(d) the TRE comprises an ApoE-HCR1 sequence element that comprises a nucleotide sequence of SEQ ID NO: 28 or a variant of SEQ ID NO: 28 that differs by 1, 2, or 3 nucleotides and the HNF1 binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
(e) the TRE comprises an ApoE-HCR1 sequence element that comprises a nucleotide sequence of SEQ ID NO: 27 or a variant of SEQ ID NO: 27 that differs by 1, 2, or 3 nucleotides and the HNF1 binding site is 3′ of the ApoE-HCR1 sequence element that comprises a nucleotide sequence of SEQ ID NO: 27 or a variant of SEQ ID NO: 27 that differs by 1, 2, or 3 nucleotides; and/or
(f) the TRE comprises an AMBP sequence element that comprises a nucleotide sequence of SEQ ID NO: 33 or a variant of SEQ ID NO: 33 that differs by 1, 2, or 3 nucleotides and the HNF1 binding site is 3′ of the AMBP sequence element that comprises a nucleotide sequence of SEQ ID NO: 33 or a variant of SEQ ID NO: 33 that differs by 1, 2, or 3 nucleotides; and/or
(g) the TRE comprises an AMBP sequence element that comprises a nucleotide sequence of SEQ ID NO: 32 or a variant of SEQ ID NO: 32 that differs by 1, 2, or 3 nucleotides and the HNF1 binding site is 3′ of the AMBP sequence element that comprises a nucleotide sequence of SEQ ID NO: 32 or a variant of SEQ ID NO: 32 that differs by 1, 2, or 3 nucleotides.

123. The TRE of any one of embodiments 121-122, wherein the TRE promotes equivalent or greater expression than a corresponding TRE not comprising the HNF1 binding site.

124. The TRE of any one of embodiments 121-123, wherein the TRE comprises an HNF6 binding site, an HNF4a binding site, and a TATA box and the TRE promotes equivalent or greater expression than a corresponding TRE not comprising the HNF6 binding site, the HNF4a binding site, and the TATA box.

125. The TRE of any one of embodiments 107-124, wherein the TRE further comprises an HNF3 binding site.

126. The TRE of embodiment 125, wherein the TRE comprises an HNF3 binding site, and:
(a) the TRE comprises a hAAT sequence element that comprises a nucleotide sequence of SEQ ID NO: 30 or a variant of SEQ ID NO: 30 that differs by 1, 2, or 3 nucleotides and the HNF3 binding site is 5′ of the hAAT sequence element that comprises a nucleotide sequence of SEQ ID NO: 30 or a variant of SEQ ID NO: 30 that differs by 1, 2, or 3 nucleotides; and/or
(b) the TRE comprises a hAAT sequence element that comprises a nucleotide sequence of SEQ ID NO: 31 or a variant of SEQ ID NO: 31 that differs by 1, 2, or 3 nucleotides and the HNF3 binding site is 5′ of the hAAT sequence element that comprises a nucleotide sequence of SEQ ID NO: 31 or a variant of SEQ ID NO: 31 that differs by 1, 2, or 3 nucleotides; and/or
(c) the TRE comprises a hAAT sequence element that comprises a nucleotide sequence of SEQ ID NO: 29 or a variant of SEQ ID NO: 29 that differs by 1, 2, or 3 nucleotides and the HNF3 binding site is 5′ of the hAAT sequence element that comprises a nucleotide sequence of SEQ ID NO: 29 or a variant of SEQ ID NO: 29 that differs by 1, 2, or 3 nucleotides; and/or
(d) the TRE comprises an ApoE-HCR1 sequence element that comprises a nucleotide sequence of SEQ ID NO: 28 or a variant of SEQ ID NO: 28 that differs by 1, 2, or 3 nucleotides and the HNF3 binding site is 3′ of the ApoE-HCR1 sequence element that comprises a nucleotide sequence of SEQ ID NO: 28 or a variant of SEQ ID NO: 28 that differs by 1, 2, or 3 nucleotides; and/or
(e) the TRE comprises an ApoE-HCR1 sequence element that comprises a nucleotide sequence of SEQ ID NO: 27 or a variant of SEQ ID NO: 27 that differs by 1, 2, or 3 nucleotides and the HNF3 binding site is 3′ of the ApoE-HCR1 sequence element that comprises a nucleotide sequence of SEQ ID NO: 27 or a variant of SEQ ID NO: 27 that differs by 1, 2, or 3 nucleotides; and/or
(f) the TRE comprises an AMBP sequence element that comprises a nucleotide sequence of SEQ ID NO: 33 or a variant of SEQ ID NO: 33 that differs by 1, 2, or 3 nucleotides and the HNF3 binding site is 3′ of the AMBP sequence element that comprises a nucleotide sequence of SEQ ID NO: 33 or a variant of SEQ ID NO: 33 that differs by 1, 2, or 3 nucleotides; and/or
(g) the TRE comprises an AMBP sequence element that comprises a nucleotide sequence of SEQ ID NO: 32 or a variant of SEQ ID NO: 32 that differs by 1, 2, or 3 nucleotides and the HNF3 binding site is 3′ of the AMBP sequence element that comprises a nucleotide sequence of SEQ ID NO: 32 or a variant of SEQ ID NO: 32 that differs by 1, 2, or 3 nucleotides.

127. The TRE of any one of embodiments 125-126, wherein the TRE promotes equivalent or greater expression than a corresponding TRE not comprising the HNF3 binding site.

128. The TRE of any one of embodiments 125-127, wherein the TRE comprises an HNF3 binding site and a TATA box and the TRE promotes equivalent or greater expression than a corresponding TRE not comprising the HNF3 binding site and the TATA box.

129. The TRE of any one of the preceding embodiments, wherein the TRE promotes gene expression in cells from at least one other organ or tissue at a level less than 40%, less than 30%, less than 25%, less than 15%, less than 10%, or less than 5% of the level that the TRE promotes gene expression in liver cells.

130. The TRE of embodiment 129, wherein the cells from at least one other organ or tissue one or more of kidney cells, pancreatic cells, breast cells, neuroblastoma cells, lung cells, cardiomyocyte cells, and early B cells.

131. The TRE of embodiment 129 or 130, wherein the cells from at least one other organ or tissue are kidney cells, pancreatic cells, breast cells, neuroblastoma cells, lung cells, cardiomyocyte cells, and early B cells.

132. The TRE of any one of embodiments 129 to 131, wherein the cells from at least one other organ or tissue are:
(i) HEK293T cells:
(ii) 697 cells:
(iii) BxPC-3 cells:
(iv) MCF7 cells:

(v) 1643 cells:

(vi) MRC-9 cells; and/or
(vii) AC-16 cells.

133. The TRE of any one of embodiments 129 to 132, wherein the level of gene expression that is promoted is measured by transducing the liver cells and the cells from at least one other organ or tissue with a polynucleotide comprising the TRE and GFP, and measuring the number of GFP positive cells, wherein the number of GFP positive cells is a measure of the level of gene expression.

134. The TRE of any one of embodiments 129 to 133, wherein the level of gene expression that is promoted is measured by transducing the liver cells and the cells from at least one other organ or tissue with a polynucleotide comprising the TRE and GFP, and measuring the mean fluorescence intensity, wherein the mean fluorescence intensity is a measure of the level of gene expression.

135. An expression cassette comprising a TRE of any one of the preceding embodiments operably linked to a transgene.

136. The expression cassette of embodiment 135, wherein the expression cassette is 2.2 kbp-4.2 kbp, 3.0 kbp-4.2 kbp, or 3.8 kbp-4.2 kbp in length.

137. A viral particle comprising a vector genome comprising a TRE of any one of embodiments 1-134 or an expression cassette of embodiment 135 or 136.

138. The viral particle of embodiment 137, wherein the viral particle is a recombinant adeno-associated virus (AAV) particle.

139. An AAV vector comprising a vector genome comprising a TRE of any one of embodiments 1-134 or the expression cassette of embodiment 135 or 136

140. The viral particle or AAV vector of any one of embodiments 137-139, wherein the vector genome comprises the expression cassette and a 5′ UTR and/or a 3′ UTR.

141. The viral particle or AAV vector of any one of embodiments 137-140, wherein the expression cassette further comprises a nucleic acid sequence encoding a poly A sequence.

142. The viral particle or AAV vector of any one of embodiments 137-141, wherein the expression cassette further comprises a nucleic acid sequence encoding a signal peptide.

143. The viral particle or AAV vector of any one of embodiments 137-142, wherein the vector genome is less than 4.9 kbp in length, optionally wherein the vector genome is more than 2.5 kbp in length.

144. The viral particle or AAV vector of any one of embodiments 137-143, wherein the vector genome is 2.2 kbp-5.0 kbp, 3 kbp-4.8 kbp, 3.5 kbp-4.8 kbp, 4.0 kbp-5.8 kbp, or around 4.7 kbp in length.

145. The viral particle or AAV vector of any one of embodiments 137-144, wherein the transgene encodes a protein or a non-translated RNA which is associated with to a genetic disorder.

146. The viral particle or AAV vector of any one of embodiments 137-145, wherein the transgene does not encode a FVIII polypeptide.

147. A pharmaceutical composition comprising the viral particle or AAV vector of any one of embodiments 137-146 and a pharmaceutically acceptable excipient.

148. A method of treatment comprising administering an effective amount of the viral particle or AAV vector of any one of embodiments 137-146 or composition of embodiment 147 to a patient.

149. The method of embodiment 148, wherein the method of treatment is a method of gene therapy.

150. The method of embodiment 148 or embodiment 149, wherein the method of treatment is a method of treating a genetic disorder, optionally a method of treating a genetic disorder by gene therapy.

151. Use of the viral particle or AAV vector of any one of embodiments 137-146, or composition of embodiment 147, in the manufacture of a medicament for treating a disease.

152. The viral particle or AAV vector of any one of embodiments 137-146 or composition of embodiment 147, for use in a method of treating a disease.

153. The viral particle, AAV vector for use or use of embodiment 151 or 152, wherein treating the disease comprises administering an effective amount of the viral particle or AAV vector of any one of embodiments 137-146 or the composition of embodiment 147 to a patient in need thereof.

154. The viral particle or AAV vector for use or use of any one of embodiments 151-153, wherein the disease is a genetic disorder.

155. The viral particle or AAV vector for use or use of any one of embodiments 151-153, wherein the disease is a complement-mediated disorder.

1. An adeno-associated virus (AAV) vector comprising a vector genome comprising a transcription regulatory element (TRE) that:
(a) comprises a human alpha-1-antitrypsin (hAAT) sequence element;
(b) comprises a human alpha-1-microglobulin/bikunin precursor (AMBP) sequence element; and
(c) promotes greater expression than hepatic control region-hAAT (HCR-hAAT), wherein the vector genome is between 2.2 and 5.0 kbp in length.

2. The AAV vector of claim 1, wherein the TRE promotes greater expression than FRE72.

3. The AAV vector of claim 1, wherein the TRE is greater than 800 nucleotides in length.

4. The AAV vector of claim 1, wherein the hAAT sequence element comprises a nucleotide sequence of SEQ ID NO: 29 or a variant of SEQ ID NO: 29 that differs by 1, 2, or 3 nucleotides.

5. The AAV vector of claim 1, wherein the AMBP sequence element comprises a nucleotide sequence of SEQ ID NO: 32 or a variant of SEQ ID NO: 32 that differs by 1, 2, or 3 nucleotides.

6. The AAV vector of claim 1, wherein the AMBP sequence element is 5′ of the hAAT sequence element.

7. The AAV vector of claim 1, wherein the TRE is liver specific.

8. The AAV vector of claim 1, wherein:
(a) the TRE comprises an apolipoprotein E-hepatic control region-1 (ApoE-HCR1) sequence element, or
(b) the TRE comprises an ApoE-HCR1 sequence element and wherein:
(i) the ApoE-HCR1 sequence element comprises a nucleotide sequence of SEQ ID NO: 27 or a variant of SEQ ID NO: 27 that differs by 1, 2, or 3 nucleotides; or
(ii) (I) the ApoE-HCR1 sequence element is 5′ of both the hAAT sequence element and the AMBP sequence element, or
(II) the ApoE-HCR1 sequence element is 5′ of the hAAT sequence element and 3′ of the AMBP sequence element.

9. (canceled)

10. (canceled)

11. (canceled)

12. The AAV vector of claim 1, wherein:
(a) the TRE comprises an aldolase B (ALDOB) sequence element; or
(b) the TRE comprises an ALDOB sequence element, and wherein:
(i) the ALDOB sequence element comprises a nucleotide sequence of SEQ ID NO: 37 or a variant of SEQ ID NO: 37 that differs by 1, 2, or 3 nucleotides, or
(ii) the ALDOB sequence element is 5′ of both the hAAT sequence element and the AMBP sequence element.

13. (canceled)

14. (canceled)

15. The AAV vector of claim 1, wherein the TRE comprises:
(a) a nucleotide sequence having at least 80% sequence identity to SEQ ID NO: 9; or
(b) a nucleotide sequence of SEQ ID NO: 9.

16. The AAV vector of claim 1, wherein the TRE comprises:
(a) a nucleotide sequence having at least 80% sequence identity to SEQ ID NO: 5; or
(b) a nucleotide sequence of SEQ ID NO: 5.

17. The AAV vector of claim 1, wherein the TRE promotes gene expression in cells from at least one other organ or tissue at a level less than 40% of the level that the TRE promotes gene expression in liver cells, wherein:
(a) the cells from the at least one other organ or tissue are kidney cells, pancreatic cells, breast cells, neuroblastoma cells, lung cells, cardiomyocyte cells, early B cells, or any combination thereof;
(b) the cells from the least one other organ or tissue are:
(i) HEK293T cells;
(ii) 697 cells;
(iii) BxPC-3 cells;
(iv) MCF7 cells;
(v) 1643 cells;
(vi) MRC-9 cells;
(vii) AC-16 cells; or
(viii) any combination of (i)-(vii);
(c) the level of gene expression that is promoted is measured by transducing the liver cells and the cells from the at least one other organ or tissue with a polynucleotide comprising the TRE and GFP, and measuring the number of GFP positive cells, wherein the number of GFP positive cells is a measure of the level of gene expression; or
(d) the level of gene expression that is promoted is measured by transducing the liver cells and the cells from the at least one other organ or tissue with a polynucleotide comprising the TRE and GFP, and measuring the mean fluorescence intensity, wherein the mean fluorescence intensity is a measure of the level of gene expression.

18. The AAV vector of claim 1, wherein the TRE is comprised within an expression cassette.

19. The AAV vector of claim 18, wherein the expression cassette is 2.2 kbp-4.2 kbp in length.

20. The AAV vector of claim 1, wherein the vector genome is less than 4.9 kbp in length.

21. The AAV vector of claim 1, wherein the vector genome is 3 kbp-4.8 kbp in length.

22. The AAV vector of claim 1, wherein the transgene encodes a protein or a non-translated RNA which is associated with a genetic disorder.

23. A pharmaceutical composition comprising the AAV vector of claim 1 and a pharmaceutically acceptable excipient.

24. The AAV vector of claim 1, for use in a method of treating a disease.

25. (canceled)

26. The AAV vector of claim 1, wherein the TRE has greater expression compared to HCR-hAAT or FRE72 in liver cells.


SEQUENCE LISTING (141 sequences)

SEQ ID NO: 1 - DNA, 119 bp, synthetic construct
  misc_feature 1..119: note = Synthetic Polynucleotide
  source 1..119: mol_type = other DNA
cccagccagtggacttagcccctgtttgctcctccgataactggggtgaccttggttaatattcaccagcagcctccccctcagcttcaggcaccaccactgacctgggacagtgaatc

SEQ ID NO: 2 - DNA, 747 bp, synthetic construct
  misc_feature 1..747: note = Synthetic Polynucleotide
  source 1..747: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 3 - DNA, 576 bp, synthetic construct
  misc_feature 1..576: note = Synthetic Polynucleotide
  source 1..576: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 4 - DNA, 898 bp, synthetic construct
  misc_feature 1..898: note = Synthetic Polynucleotide
  source 1..898: mol_type = other DNA
caggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctctacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 5 - DNA, 898 bp, synthetic construct
  misc_feature 1..898: note = Synthetic Polynucleotide
  source 1..898: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 6 - DNA, 573 bp, synthetic construct
  misc_feature 1..573: note = Synthetic Polynucleotide
  source 1..573: mol_type = other DNA
tgcctccccgtgttcctgctctttgtccctctgtcctacttagactaatatttgccttgggtactgcaaacaggaaatgggggagggacaggaggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatcc

SEQ ID NO: 7 - DNA, 1618 bp, synthetic construct
  misc_feature 1..1618: note = Synthetic Polynucleotide
  source 1..1618: mol_type = other DNA
cacaatttttatagacttctcatcatgttttttttttccaatatttgccctcccctttctaccttgacctagtagggaggtgtttattcaacacctctgattggctcataaacaaccagtccttaaaatcatgccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtcagtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 8 - DNA, 1363 bp, synthetic construct
  misc_feature 1..1363: note = Synthetic Polynucleotide
  source 1..1363: mol_type = other DNA
ggaggggtgttaattaaaagcttgatatgggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtcatgcctccccgtgttcctgctctttgtccctctgtcctacttagactaatatttgccttgggtactgcaaacaggaaatgggggagggacaggacccagccagtggacttagcccctgtttgctcctccgataactggggtgaccttggttaatattcaccagcagcctcccccggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 9 - DNA, 1484 bp, synthetic construct
  misc_feature 1..1484: note = Synthetic Polynucleotide
  source 1..1484: mol_type = other DNA
gggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtcacaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggccg

SEQ ID NO: 10 - DNA, 903 bp, synthetic construct
  misc_feature 1..903: note = Synthetic Polynucleotide
  source 1..903: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggatataaacgaggacagggccctgtctcctcagcttcaggcaccaccactgacctgggacagtgaatgatccccctgatctgcggcc

SEQ ID NO: 11 - DNA, 933 bp, synthetic construct
  misc_feature 1..933: note = Synthetic Polynucleotide
  source 1..933: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctccccctattgattgttgcccctctggatccactgcttaaatacggacgaggacagggccctgtctcctcagcttcaggcaccaccactgacctgggacagtgaatgatccccctgatctgcggcc

SEQ ID NO: 12 - DNA, 943 bp, synthetic construct
  misc_feature 1..943: note = Synthetic Polynculeotide
  source 1..943: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggttaataatctggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctccccctattgattgttgcccctctggatccactgcttaaatacggacgaggacagggccctgtctcctcagcttcaggcaccaccactgacctgggacagtgaatgatccccctgatctgcggcc

SEQ ID NO: 13 - DNA, 913 bp, synthetic construct
  misc_feature 1..913: note = Synthetic Polynucleotide
  source 1..913: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggctgtttgctcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 14 - DNA, 915 bp, synthetic construct
  misc_feature 1..915: note = Synthetic Polynucleotide
  source 1..915: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 15 - DNA, 909 bp, synthetic construct
  misc_feature 1..909: note = Synthetic Polynucleotide
  source 1..909: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 16 - DNA, 907 bp, synthetic construct
  misc_feature 1..907: note = Synthetic Polynucleotide
  source 1..907: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctctggcccttgccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 17 - DNA, 909 bp, synthetic construct
  misc_feature 1..909: note = Synthetic Polynculeotide
  source 1..909: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggttattgatattcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 18 - DNA, 907 bp, synthetic construct
  misc_feature 1..907: note = Synthetic Polynucleotide
  source 1..907: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 19 - DNA, 907 bp, synthetic construct
  misc_feature 1..907: note = Synthetic Polynucleotide
  source 1..907: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacatggcccttgccagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 20 - DNA, 913 bp, synthetic construct
  misc_feature 1..913: note = Synthetic Polynucleotide
  source 1..913: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctccccctatataaatattgattgttgcccctctggatccactgcttaaatacggacgaggacagggccctgtctcctcagcttcaggcaccaccactgacctgggacagtgaatgatccccctgatctgcggcc

SEQ ID NO: 21 - DNA, 919 bp, synthetic construct
  misc_feature 1..919: note = Synthetic Polynucleotide
  source 1..919: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctcttattgatattccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtggcccttgcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 22 - DNA, 919 bp, synthetic construct
  misc_feature 1..919: note = Synthetic Polynucleotide
  source 1..919: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctctggcccttgccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggttattgatattcgtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 23 - DNA, 915 bp, synthetic construct
  misc_feature 1..915: note = Synthetic Polynucleotide
  source 1..915: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccatataaagccagtggacttagcccctgtttgctcctccgataactggggtgaccttggttaatattcaccagcagcctcccccttattgatattcgttgcccctctggatccactgcttaaatacggacgaggacagggccctgtctcctcagcttcaggcaccaccactgacctgggacagtgaatgatccccctgatctgcggcc

SEQ ID NO: 24 - DNA, 909 bp, synthetic construct
  misc_feature 1..909: note = Synthetic Polynucleotide
  source 1..909: mol_type = other DNA
ttattgatattcggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 25 - DNA, 907 bp, synthetic construct
  misc_feature 1..907: note = Synthetic Polynucleotide
  source 1..907: mol_type = other DNA
tggcccttgcggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 26 - DNA, 907 bp, synthetic construct
  misc_feature 1..907: note = Synthetic Polynucleotide
  source 1..907: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctccccctttattgatattcgcccctctggatccactgcttaaatacggacgaggacagggccctgtctcctcagcttcaggcaccaccactgacctgggacagtgaatgatccccctgatctgcggcc

SEQ ID NO: 27 - DNA, 322 bp, synthetic construct
  misc_feature 1..322: note = Synthetic Polynucleotide
  source 1..322: mol_type = other DNA
caggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 28 - DNA, 117 bp, synthetic construct
  misc_feature 1..117: note = Synthetic Polynucleotide
  source 1..117: mol_type = other DNA
ccctaaaatgggcaaacattgcaagcagcaaacagcaaacacacagccctccctgcctgctgaccttggagctggggcagaggtcagacacctctctgggcccatgccacctccaac

SEQ ID NO: 29 - DNA, 426 bp, synthetic construct
  misc_feature 1..426: note = Synthetic Polynucleotide
  source 1..426: mol_type = other DNA
gtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgatccccctgatctgcggcc

SEQ ID NO: 30 - DNA, 73 bp, synthetic construct
  misc_feature 1..73: note = Synthetic Polynucleotide
  source 1..73: mol_type = other DNA
agtggacttagcccctgtttgctcctccgataactggggtgaccttggttaatattcaccagcagcctccccc

SEQ ID NO: 31 - DNA, 80 bp, synthetic construct
  misc_feature 1..80: note = Synthetic Polynucleotide
  source 1..80: mol_type = other DNA
cccagccagtggacttagcccctgtttgctcctccgataactggggtgaccttggttaatattcaccagcagcctccccc

SEQ ID NO: 32 - DNA, 149 bp, synthetic construct
  misc_feature 1..149: note = Synthetic Polynucleotide
  source 1..149: mol_type = other DNA
ggttaatttttaaaaagcagtcaaaagtccaagtggcccttgcgagcatttactctctctgtttgctctggttaataatctcaggagcacaaacattcctggaggcaggagaagaaatcaacatcctggacttatcctctgggcctctc

SEQ ID NO: 33 - DNA, 93 bp, synthetic construct
  misc_feature 1..93: note = Synthetic Polynucleotide
  source 1..93: mol_type = other DNA
ttaatttttaaaaagcagtcaaaagtccaagtggcccttgcgagcatttactctctctgtttgctctggttaataatctcaggagcacaaaca

SEQ ID NO: 34 - DNA, 134 bp, synthetic construct
  misc_feature 1..134: note = Synthetic Polynucleotide
  source 1..134: mol_type = other DNA
cacaatttttatagacttctcatcatgttttttttttccaatatttgccctcccctttctaccttgacctagtagggaggtgtttattcaacacctctgattggctcataaacaaccagtccttaaaatcatgc

SEQ ID NO: 35 - DNA, 12 bp, synthetic construct
  misc_feature 1..12: note = Synthetic Polynucleotide
  source 1..12: mol_type = other DNA
aatttttataga

SEQ ID NO: 36 - DNA, 52 bp, synthetic construct
  misc_feature 1..52: note = Synthetic Polynucleotide
  source 1..52: mol_type = other DNA
gtagggaggtgtttattcaacacctctgattggctcataaacaaccagtcct

SEQ ID NO: 37 - DNA, 586 bp, synthetic construct
  misc_feature 1..586: note = Synthetic Polynucleotide
  source 1..586: mol_type = other DNA
gggattcaaattctgcttagctaacccaatgttgtgagttaatgcttgtctacttgggcagaagtaccttgcaaaattatctaggtagcatgtgatagaaccaggattcaaacccaggtgtgtctgacctcaaagtgtggggactttccactctactgctgctaatttttttctaaaaataatacttccgggtgcagagtttaatcttctttgtccaaggtactgtggtaagcaccttatgtacattgtctaagttaatccccacaacattttgagaaatccccattttgagataggagaactgaggctcacaatatttcagtgactggcccaaggtcacagttgttaaatggaggagctggtattgaaaaccaggtacgtgtggctctaagaccagtgtaatagttgtgttttgtttttccttgcttccttctttacttgccttcatttctagcttacactggcatgattcatctcagtgggcaatatccttaccttgaatggtggtttctttgtttgttcctgcaagaggagcacctccttggtctaactgtggatacaaataattaacaggtgtcagatgtca

SEQ ID NO: 38 - DNA, 113 bp, synthetic construct
  misc_feature 1..113: note = Synthetic Polynucleotide
  source 1..113: mol_type = other DNA
cttgcaaaattatctaggtagcatgtgatagaaccaggattcaaacccaggtgtgtctgacctcaaagtgtggggactttccactctactgctgctaatttttttctaaaaat

SEQ ID NO: 39 - DNA, 93 bp, synthetic construct
  misc_feature 1..93: note = Synthetic Polynucleotide
  source 1..93: mol_type = other DNA
tgcctccccgtgttcctgctctttgtccctctgtcctacttagactaatatttgccttgggtactgcaaacaggaaatgggggagggacagga

SEQ ID NO: 40 - DNA, 77 bp, synthetic construct
  misc_feature 1..77: note = Synthetic Polynucleotide
  source 1..77: mol_type = other DNA
tgttcctgctctttgtccctctgtcctacttagactaatatttgccttgggtactgcaaacaggaaatgggggaggg

SEQ ID NO: 41 - DNA, 1752 bp, synthetic construct
  misc_feature 1..1752: note = Synthetic Polynucleotide
  source 1..1752: mol_type = other DNA
atgaagctgctgcatgtgttcctgctgttcctgtgcttccacctgagattctgcaaggtgacctacaccagccaggaggacctggtggagaagaagtgcctggccaagaagtacacccacctgagctgtgacaaggtgttctgccagccctggcagagatgcattgagggcacctgtgtgtgcaagctgccctaccagtgccccaagaatggcacagctgtgtgtgccaccaacagaaggagcttccccacctactgccagcagaagagcctggagtgcctgcaccctggcaccaagttcctgaacaatggcacctgcacagctgagggcaagttcagtgtgagcctgaagcatggcaacacagacagtgagggcattgtggaggtgaagctggtggaccaggacaagaccatgttcatctgcaagagcagttggagcatgagagaggccaatgtggcctgcctggacctgggcttccagcaaggagctgacacccagagaaggttcaagctgagtgacctgagcatcaacagcacagagtgcctgcatgtgcactgcagaggcctggagaccagcctggctgagtgcaccttcaccaagagaaggaccatgggctaccaggactttgctgatgtggtttgctacacccagaaggctgacagccccatggatgacttcttccagtgtgtgaatggcaagtacatcagccagatgaaggcctgtgatggcatcaatgactgtggagaccagagtgatgagctgtgctgcaaggcctgccagggcaagggcttccactgcaagagtggagtgtgcatccccagccagtaccagtgcaatggagaggtggactgcatcacaggagaggatgaggtgggctgtgctggctttgccagtgtgacccaggaggagacagagatcctgacagctgacatggatgctgagagaaggagaatcaagagcctgctgcccaagctgagctgtggagtgaagaacagaatgcacatcagaaggaagagaattgtgggaggcaagagagcccagctgggagacctgccctggcaggtggccatcaaggatgccagtggcatcacctgtggaggaatctacattggaggctgctggattctgacagctgcccactgcctgagagccagcaagacccacagataccagatctggaccacagtggttgactggattcaccctgacctgaagagaattgtgattgagtatgtggacagaatcattttccatgagaactacaatgctggcacctaccagaatgacattgccctgattgagatgaagaaggatggcaacaagaaagactgtgagctgcccagaagcatccctgcctgtgtgccctggagcccctacctgttccagcccaatgacacctgtattgtcagtggatggggcagagagaaggacaatgagagagtgttcagcctgcagtggggagaggtgaagctgatcagcaactgcagcaagttctatggcaacagattctatgagaaggagatggagtgtgctggcacctatgatggcagcattgatgcctgcaagggagacagtggaggccccctggtgtgcatggatgccaacaatgtgacctatgtgtggggagtggttagctggggagagaactgtggcaagcctgagttccctggagtgtacaccaaggtggccaactactttgactggatcagctaccatgtgggcagacccttcatcagccagtacaatgtgtaa

SEQ ID NO: 42 - DNA, 335 bp, synthetic construct
  misc_feature 1..335: note = Synthetic Polynucleotide
  source 1..335: mol_type = other DNA
ccctaaaatgggcaaacattgcaagcagcaaacagcaaacacacagccctccctgcctgctgaccttggagctggggcagaggtcagacacctctctgggcccatgccacctccaactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 43 - DNA, 14 bp, Homo sapiens
  source 1..14: mol_type = genomic DNA
tagactaatatttg

SEQ ID NO: 44 - skipped

SEQ ID NO: 45 - DNA, 10 bp, Homo sapiens
  source 1..10: mol_type = genomic DNA
tatgtacatt

SEQ ID NO: 46 - DNA, 10 bp, Homo sapiens
  source 1..10: mol_type = genomic DNA
cccacaacat

SEQ ID NO: 47 - skipped

SEQ ID NO: 48 - DNA, 10 bp, Homo sapiens
  source 1..10: mol_type = genomic DNA
gattcatctc

SEQ ID NO: 49 - DNA, 10 bp, Homo sapiens
  source 1..10: mol_type = genomic DNA
ttggtctaac

SEQ ID NO: 50 - DNA, 12 bp, Homo sapiens
  source 1..12: mol_type = genomic DNA
ctttctaccttg

SEQ ID NO: 51 - DNA, 10 bp, Homo sapiens
  source 1..10: mol_type = genomic DNA
ctgtttgctc

SEQ ID NO: 52 - skipped

SEQ ID NO: 53 - DNA, 10 bp, Homo sapiens
  source 1..10: mol_type = genomic DNA
ctgtttgctc

SEQ ID NO: 54-55 - skipped (2 entries)

SEQ ID NO: 56 - DNA, 11 bp, Homo sapiens
  source 1..11: mol_type = genomic DNA
aaaagcagtca

SEQ ID NO: 57 - DNA, 11 bp, Homo sapiens
  source 1..11: mol_type = genomic DNA
aggcaggagaa

SEQ ID NO: 58 - DNA, 11 bp, Homo sapiens
  source 1..11: mol_type = genomic DNA
gagaagaaatc

SEQ ID NO: 59 - DNA, 11 bp, Homo sapiens
  source 1..11: mol_type = genomic DNA
caacatcctgg

SEQ ID NO: 60 - DNA, 11 bp, Homo sapiens
  source 1..11: mol_type = genomic DNA
acttatcctct

SEQ ID NO: 61 - DNA, 11 bp, Homo sapiens
  source 1..11: mol_type = genomic DNA
cgtgttcctgc

SEQ ID NO: 62-64 - skipped (3 entries)

SEQ ID NO: 65 - DNA, 18 bp, Homo sapiens
  source 1..18: mol_type = genomic DNA
ataatctcaggagcacaa

SEQ ID NO: 66-68 - skipped (3 entries)

SEQ ID NO: 69 - DNA, 11 bp, Homo sapiens
  source 1..11: mol_type = genomic DNA
gacttatcctc

SEQ ID NO: 70 - DNA, 18 bp, Homo sapiens
  source 1..18: mol_type = genomic DNA
tgtttattcaacacctct

SEQ ID NO: 71 - DNA, 10 bp, Homo sapiens
  source 1..10: mol_type = genomic DNA
gttaataatc

SEQ ID NO: 72 - DNA, 13 bp, Homo sapiens
  source 1..13: mol_type = genomic DNA
gactaatatttgc

SEQ ID NO: 73 - DNA, 13 bp, Homo sapiens
  source 1..13: mol_type = genomic DNA
cttgcaaaattat

SEQ ID NO: 74 - DNA, 13 bp, Homo sapiens
  source 1..13: mol_type = genomic DNA
gctgctaattttt

SEQ ID NO: 75 - DNA, 13 bp, Homo sapiens
  source 1..13: mol_type = genomic DNA
acaaataattaac

SEQ ID NO: 76 - DNA, 13 bp, Homo sapiens
  source 1..13: mol_type = genomic DNA
gttaatttttaaa

SEQ ID NO: 77 - DNA, 11 bp, Homo sapiens
  source 1..11: mol_type = genomic DNA
ggttaataatc

SEQ ID NO: 78 - DNA, 12 bp, Homo sapiens
  source 1..12: mol_type = genomic DNA
tggttaatattc

SEQ ID NO: 79 - DNA, 10 bp, Homo sapiens
  source 1..10: mol_type = genomic DNA
tgtttgctct

SEQ ID NO: 80 - DNA, 10 bp, Homo sapiens
  source 1..10: mol_type = genomic DNA
agcacaaaca

SEQ ID NO: 81-83 - skipped (3 entries)

SEQ ID NO: 84 - DNA, 11 bp, Homo sapiens
  source 1..11: mol_type = genomic DNA
agaaatcaaca

SEQ ID NO: 85 - DNA, 15 bp, Homo sapiens
  source 1..15: mol_type = genomic DNA
ctaatatttgccttg

SEQ ID NO: 86 - DNA, 13 bp, Homo sapiens
  source 1..13: mol_type = genomic DNA
gactaatatttgc

SEQ ID NO: 87 - DNA, 12 bp, Homo sapiens
  source 1..12: mol_type = genomic DNA
tctgtttgctct

SEQ ID NO: 88 - DNA, 17 bp, Homo sapiens
  source 1..17: mol_type = genomic DNA
ttcctgctctttgtccc

SEQ ID NO: 89 - skipped

SEQ ID NO: 90 - DNA, 12 bp, Homo sapiens
  source 1..12: mol_type = genomic DNA
ccctgtttgctc

SEQ ID NO: 91 - DNA, 10 bp, Homo sapiens
  source 1..10: mol_type = genomic DNA
tggcccttgc

SEQ ID NO: 92 - DNA, 15 bp, Homo sapiens
  source 1..15: mol_type = genomic DNA
agcagtcaaaagtcc

SEQ ID NO: 93 - DNA, 12 bp, Homo sapiens
  source 1..12: mol_type = genomic DNA
ttattgatattc

SEQ ID NO: 94 - skipped

SEQ ID NO: 95 - DNA, 15 bp, Homo sapiens
  source 1..15: mol_type = genomic DNA
ttttttttccaatat

SEQ ID NO: 96 - DNA, 21 bp, Homo sapiens
  source 1..21: mol_type = genomic DNA
atttactctctctgtttgctc

SEQ ID NO: 97 - DNA, 21 bp, Homo sapiens
  source 1..21: mol_type = genomic DNA
ttactctctctgtttgctctg

SEQ ID NO: 98 - DNA, 13 bp, Homo sapiens
  source 1..13: mol_type = genomic DNA
ttaataatctcag

SEQ ID NO: 99 - DNA, 17 bp, Homo sapiens
  source 1..17: mol_type = genomic DNA
tggttaatattcaccag

SEQ ID NO: 100 - DNA, 11 bp, Homo sapiens
  source 1..11: mol_type = genomic DNA
caacatcctgg

SEQ ID NO: 101 - DNA, 13 bp, Homo sapiens
  source 1..13: mol_type = genomic DNA
agtccaagtggcc

SEQ ID NO: 102 - DNA, 12 bp, Homo sapiens
  source 1..12: mol_type = genomic DNA
gtccaagtggcc

SEQ ID NO: 103 - DNA, 10 bp, Homo sapiens
  source 1..10: mol_type = genomic DNA
ctctggttaa

SEQ ID NO: 104 - DNA, 10 bp, Homo sapiens
  source 1..10: mol_type = genomic DNA
aaatggggga

SEQ ID NO: 105 - DNA, 10 bp, Homo sapiens
  source 1..10: mol_type = genomic DNA
tcaaaagtcc

SEQ ID NO: 106 - DNA, 14 bp, Homo sapiens
  source 1..14: mol_type = genomic DNA
tagactaatatttg

SEQ ID NO: 107 - DNA, 15 bp, Homo sapiens
  source 1..15: mol_type = genomic DNA
caggagaagaaatca

SEQ ID NO: 108 - DNA, 14 bp, Homo sapiens
  source 1..14: mol_type = genomic DNA
tgttaattaaaagc

SEQ ID NO: 109 - DNA, 10 bp, Homo sapiens
  source 1..10: mol_type = genomic DNA
gttaattttt

SEQ ID NO: 110 - DNA, 10 bp, Homo sapiens
  source 1..10: mol_type = genomic DNA
ttactctctc

SEQ ID NO: 111 - DNA, 10 bp, Homo sapiens
  source 1..10: mol_type = genomic DNA
cttgggtact

SEQ ID NO: 112-121 - skipped (10 entries)

SEQ ID NO: 122 - DNA, 10 bp, Homo sapiens
  source 1..10: mol_type = genomic DNA
tggaggcagg

SEQ ID NO: 123 - skipped

SEQ ID NO: 124 - DNA, 13 bp, Homo sapiens
  source 1..13: mol_type = genomic DNA
atgggggagggac

SEQ ID NO: 125-126 - skipped (2 entries)

SEQ ID NO: 127 - DNA, 11 bp, Homo sapiens
  source 1..11: mol_type = genomic DNA
attcctggagg

SEQ ID NO: 128 - DNA, 11 bp, Homo sapiens
  source 1..11: mol_type = genomic DNA
caaacaggaaa

SEQ ID NO: 129-130 - skipped (2 entries)

SEQ ID NO: 131 - DNA, 13 bp, Homo sapiens
  source 1..13: mol_type = genomic DNA
aaacattcctgga

SEQ ID NO: 132-134 - skipped (3 entries)

SEQ ID NO: 135 - DNA, 11 bp, Homo sapiens
  source 1..11: mol_type = genomic DNA
tccaagtggcc

SEQ ID NO: 136 - DNA, 11 bp, Homo sapiens
  source 1..11: mol_type = genomic DNA
agcatgtgata

SEQ ID NO: 137-138 - skipped (2 entries)

SEQ ID NO: 139 - DNA, 1726 bp, Homo sapiens
  source 1..1726: mol_type = genomic DNA
atcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 140 - protein, 736 aa, synthetic construct
  REGION 1..736: note = Synthetic Polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 141 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY